(19) United States
(12) Patent Application Publication
(10) Pub. No.: US 2025/0041258 A1
(43) Pub. Date: Feb. 6, 2025

(54) PHARMACEUTICAL COMPOSITIONS OF A GAMMA-HYDROXYBUTYRIC ACID DERIVATIVE

(71) Applicant: XWPHARMA LTD., REDWOOD CITY, CA (US)

(72) Inventors: SAMI KARABORNI, CUPERTINO, CA (US); DANIEL M. CANAFAX, HALF MOON BAY, CA (US); JIA-NING XIANG, FREMONT, CA (US); WILLIAM W. XIANG, FREMONT, CA (US)

(21) Appl. No.: 18/789,932

(22) Filed: Jul. 31, 2024

Related U.S. Application Data
(60) Provisional application No. 63/529,880, filed on Jul. 31, 2023.

(51) Int. Cl. A61K 31/223; A61K 9/16

(52) U.S. Cl. CPC A61K 31/223; A61K 9/1611; A61K 9/1617; A61K 9/1623; A61K 9/1635; A61K 9/1652

(57) ABSTRACT

Pharmaceutical compositions including a pharmaceutical granulation comprising 4-((L-valyl)oxy) butanoic acid and an excipient granulation are disclosed. When the pharmaceutical compositions are combined and mixed with water the excipient granules rapidly dissolve to form a viscous solution with a pharmaceutical granulation suspended in the viscous solution. The pharmaceutical granulations provide palatable oral pharmaceutical compositions.

This application claims the benefit under 35 U.S.C. § 119 (e) of U.S. Provisional Application No. 63/529,880 filed on Jul. 31, 2023, which is incorporated by reference in its entirety.

### FIELD

The disclosure relates to excipient-containing granulations, pharmaceutical compositions comprising the excipient-containing granulations, and use of the pharmaceutical compositions to prepare oral formulations of 4-((L-valyl)oxy) butanoic acid.

### BACKGROUND

Active pharmaceutically active ingredients can be administered using pharmaceutical granulations. To improve the palatability of a pharmaceutical composition administered to a patient a pharmaceutical granulation can be dispersed in a viscous solution. It can be convenient to provide viscosifying agents and other excipients along with the pharmaceutical granulation such that the viscosifying agent, other excipients, and the pharmaceutical granulation can be combined with water to provide an oral pharmaceutical composition.

### SUMMARY

According to the present invention, a pharmaceutical composition comprises a pharmaceutical granulation comprising 4-((L-valyl)oxy) butanoic acid; and an excipient granulation.

According to the present invention, a pharmaceutical product comprises a pharmaceutical composition according to the present invention.

According to the present invention, a unit dose comprises a pharmaceutical composition according to the present invention.

According to the present invention, a method of using a pharmaceutical composition according to the present invention or a pharmaceutical product according to the present invention to prepare an oral pharmaceutical composition for oral administration comprises combining the pharmaceutical composition with an aqueous solution; and mixing the pharmaceutical composition to dissolve the immediate release pharmaceutical granulation and the excipient granulation to form a viscous oral pharmaceutical composition.

According to the present invention, an oral pharmaceutical composition prepared by combining and mixing a pharmaceutical composition according to the present invention with water.

According to the present invention, a method of treating a disease in a patient comprising orally administering to a patient in need of such treatment a therapeutically effective amount of an oral pharmaceutical composition according to the present invention.

### BRIEF DESCRIPTION OF THE DRAWINGS

Those skilled in the art will understand that the drawings described herein are for illustration purposes only. The drawings are not intended to limit the scope of the present disclosure.

FIG. 1 shows an example of a dissolution profile for an immediate release pharmaceutical granulation.

FIG. 2 shows an example of a dissolution profile for a modified release pharmaceutical granulation.

FIG. 3 shows an example of a dissolution profile for a pharmaceutical granulation comprising an immediate release pharmaceutical granulation and a modified release pharmaceutical granulation.

FIG. 4 shows an example of a particle size distribution of an excipient granulation.

FIG. 5 shows an example of the reconstitution viscosity of solutions prepared using excipient granulations provided by the present disclosure.

FIG. 6 shows an example of the reconstitution luminosity of solutions prepared using excipient granulations provided by the present disclosure.

FIG. 7 shows an example of a dissolution profile for a pharmaceutical composition comprising an immediate release pharmaceutical granulation, a modified release pharmaceutical granulation, and an excipient granulation.

FIG. 8 shows an example of the reconstitution viscosity for a pharmaceutical composition comprising an immediate release pharmaceutical granulation, a modified release pharmaceutical granulation, and an excipient granulation.

FIG. 9 shows the γ-hydroxybutyric acid plasma pharmacokinetic profile following oral administration of different doses of an oral pharmaceutical granulation comprising an immediate release pharmaceutical granulation and a modified release pharmaceutical granulation.

FIGS. 10A and 10B show the dissolution profiles of pharmaceutical granulations following storage at 5±3° C. (FIG. 9A) and at 25±2° C./60% RH (FIG. 9B) as described in Example 6.

FIGS. 11A and 11B show the dissolution profiles of pharmaceutical compositions comprising an excipient granulation and a pharmaceutical granulation following storage at 5±3° C. (FIG. 10A) and at 25±2° C./60% RH (FIG. 10B) as described in Example 6.

### DETAILED DESCRIPTION

For purposes of the following detailed description, it is to be understood that embodiments provided by the present disclosure may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

“Immediate release” refers to a composition that releases at least 80% of 4-((L-valyl)oxy) butanoic acid within 1 hour when tested in a dissolution apparatus 2 according to USP 38 in a 0.1 N HCl dissolution medium at a temperature of 37° C. and a paddle speed of 75 rpm.

An immediate release composition or formulation can release substantially all of a pharmaceutically active ingredient into the gastrointestinal tract of a patient within less than 1 hour following oral administration, such as within less than 50 minutes, within less than 40 minutes, within less than 30 minutes, within less than 20 minutes, or within less than 10 minutes following oral administration. For example, an immediate release dosage form can release greater than 90%, greater than 95%, or greater than 98% of the pharmaceutically active ingredient such as 4-((L-valyl)oxy) butanoic acid) in the pharmaceutical composition into the gastrointestinal tract within less than 1 hour, such as within less than 50 minutes, less than 40 minutes, less than 30 minutes, less than 20 minutes, or less than 10 minutes, following oral administration. Immediate release pharmaceutical compositions can be appropriate to administer pharmaceutically active ingredients that are absorbed into the systemic circulation from the upper portion of the gastrointestinal tract.

“Modified release” pharmaceutical compositions and formulations can include controlled release formulations, delayed release formulations, extended-release formulations, sustained release formulations, timed release formulations, pulsatile release formulations, and pH-dependent release formulations. These formulations are intended to release a pharmaceutically active ingredient from the pharmaceutical composition at a desired rate and/or at a desired time following oral administration by a patient and/or at a certain location or locations within the gastrointestinal tract and/or at a certain pH within the gastrointestinal tract. The United States Pharmacopeia (USP) defines a modified release system as one in which the time course or location of drug release or both, are chosen to accomplish objectives of therapeutic effectiveness or convenience not fulfilled by immediate release dosage forms. A modified release oral dosage form can include extended release and delayed-release components. A delayed release dosage form is one that releases a drug all at once at a time other than promptly after administration. A modified release formulation can include delayed-release using enteric coatings, site-specific or timed release such as for colonic delivery, extended-release including, for example, formulations capable of providing zero-order, first-order, or biphasic release profiles, and programmed release such as pulsatile and delayed extended release.

“Patient” refers to a mammal, for example, a human.

“Pharmaceutically acceptable” refers to approved or approvable by a regulatory agency of the Federal or a state government or listed in the U.S. Pharmacopoeia (USP) or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids and one or more protonable modified release groups such as primary, secondary, or tertiary amines within the parent compound. Examples of suitable inorganic acids include hydrochloric acid, hydrobromic acid, sulfuric acid, nitric acid, phosphoric acid, and the like. A salt can be formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid. 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. A salt can be formed when one or more acidic protons present in the parent compound are replaced by a metal ion, e.g., an alkali metal ion, an alkaline earth ion, or an aluminum ion, or combinations thereof; or coordinates with an organic base such as ethanolamine, diethanolamine, triethanolamine, N-methylglucamine, and the like. A pharmaceutically acceptable salt can be the hydrochloride salt. A pharmaceutically acceptable salt can be the sodium salt. In compounds having two or more ionizable groups, a pharmaceutically acceptable salt can comprise one or more counterions, such as a bi-salt, for example, a dihydrochloride salt.

The term “pharmaceutically acceptable salt” includes hydrates and other solvates, as well as salts in crystalline or non-crystalline form. Where a particular pharmaceutically acceptable salt is disclosed, it is understood that the particular salt (e.g., a hydrochloride salt) is an example of a salt, and that other salts may be formed using techniques known to one of skill in the art. Additionally, one of skill in the art would be able to convert the pharmaceutically acceptable salt to the corresponding compound, free base and/or free acid, using techniques generally known in the art.

“Prodrug” refers to a derivative of a drug molecule that requires a transformation within the body to provide the active drug. Prodrugs are frequently, although not necessarily, pharmacologically inactive until converted to the parent drug. Prodrugs may be obtained by bonding a promoiety (defined herein) typically via a modified release group, to a drug.

“Curing” a disease refers to eliminating the disease or disorder or eliminating a symptom of the disease or disorder.

“Treating” or “treatment” of a disease or disorder refers to reducing the severity of one or more clinical symptom of the disease or disorder, delaying the onset of one or more clinical symptoms of the disease or disorder, and/or mitigating one or more clinical symptoms of the disease or disorder. “Treating” or “treatment” of a disease or disorder refers to inhibiting the disease or disorder or one or more clinical symptoms of the disease or disorder, arresting the development of the disease or disorder or one or more clinical symptoms of the disease or disorder, relieving the disease or disorder or one or more clinical symptoms of the disease or disorder, causing the regression of the disease or disorder or one or more clinical symptoms of the disease or disorder, and/or stabilization of the disease or disorder or one or more clinical symptoms of the disease or disorder, “Treating” or “treatment” of a disease or disorder refers to producing a clinically beneficial effect without curing the underlying disease or disorder.

“Therapeutically effective amount” refers to the amount of a pharmaceutically active ingredient such as 4-((L-valyl)oxy) butanoic acid or γ-hydroxybutyric acid, when administered to a patient for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the patient to be treated, and the judgment of the prescribing physician. A therapeutically effective amount in any given instance may be ascertained by those skilled in the art or capable of determination by routine experimentation or based on the judgment of a prescribing physician or medical professional.

“Therapeutically effective dose” refers to a dose that provides effective treatment of a disease or disorder in a patient. A therapeutically effective dose of 4-((L-valyl)oxy) butanoic acid or γ-hydroxy butyric acid may vary from patient to patient and may depend upon factors such as the medical condition of the patient, the severity of the disease and the route of delivery. A therapeutically effective dose may be determined in accordance with routine pharmacological procedures known to those skilled in the art.

“Excipient” refers to a diluent, vehicle or carrier with which a compound is administered to a patient. An excipient can be a pharmaceutically acceptable vehicle. Pharmaceutically acceptable excipients are known in the art.

Reconstitution viscosity or the viscosity of an aqueous solution prepared using excipient granules can be determined using a Brookfield digital viscometer Model LVDV-1+ equipped with a Brookfield SC4-18 spindle at a spindle speed of 30 RPM. Excipient granules such as 1.2 g of the excipient granules can be added to water such as 40 mL of water in a 50 mL beaker and mixed for a specified time such as for 30, 60, 90, 120, 150, or 180 seconds. Mixing can be stopped and the viscosity measured. Reconstitution viscosity is typical measured at a temperature from 20° C. to 25° C., such as 23° C.

Bulk density can be determined according to USP 616, Method 1.

Tapped bulk density can be determined according to USP 616.

Specific surface area can be determined by laser diffraction.

The Hausner Ratio can be determined according to USP <1174>. The Hausner ratio is the ratio of the tapped density to the bulk density.

The compressibility index is the 100×[(tapped density−bulk density)/tapped density].

A particle size distribution or the average particle size of a granulation can be determined by laser diffraction or by sieve analysis.

The Flodex value can be determined according to USP <1174>.

Luminosity can be determined using a Technical Color Solutions Datacolor 650® instrument.

Friability can be measured using a sonic sifter. Friability is defined as the number of microparticles having a diameter less than 75 μm that are generated by subjecting a granulation to a sonic sifter operated at a vibration amplitude of 8 corresponding to 3,600 sonic energy pulses per minute for at least 2 minutes.

A “pharmaceutical granulation” refers to a plurality of pharmaceutical granules where the pharmaceutical granules comprise an active pharmaceutical ingredient.

An “excipient granulation” refers to a plurality of excipient granules where the excipient granules do not comprise an active pharmaceutical ingredient.

“Pharmaceutical composition” refers to a composition comprising an excipient granulation and an active pharmaceutical ingredient. The active pharmaceutical ingredient can be in the form of a pharmaceutical granulation.

“Oral pharmaceutical composition” refers to a pharmaceutical composition that is intended to be orally ingested by a patient.

Reference is now made to pharmaceutical compositions comprising a pharmaceutical granulation and an excipient granulation, and oral pharmaceutical compositions prepared using the pharmaceutical granulations. The disclosed pharmaceutical compositions and uses are not intended to be limiting of the claims. To the contrary, the claims are intended to cover all alternatives, modifications, and equivalents.

Pharmaceutical compositions provided by the present disclosure comprise a pharmaceutical granulation and an excipient granulation. A pharmaceutical granulation can comprise an immediate release pharmaceutical granulation and a modified release pharmaceutical granulation.

A pharmaceutical composition comprising immediate release and modified release granulation can independently be provided in one or more packages such as one or more sachets or one or more stick packs.

At the time of use, the contents of the one or more packages including the immediate release granulation, modified release granulation, and the excipient can be combined and mixed with water or an aqueous solution to provide a viscous solution containing the at least partially dissolved immediate release granulation and with the modified release granulation suspended in the viscous solution. The viscous solution comprising the fully dissolved contents of the excipient granulation, the at least partially dissolved immediate release granulation, and a suspension of modified release pharmaceutical granulation provides an oral pharmaceutical composition suitable for oral ingestion by a patient.

A pharmaceutical composition provided by the present disclosure can comprise an immediate release pharmaceutical granulation comprising 4-((L-valyl)oxy) butanoic acid or a pharmaceutically acceptable salt thereof, a modified release granulation comprising 4-((L-valyl)oxy) butanoic acid or a pharmaceutically acceptable salt thereof, and an excipient granulation.

A pharmaceutical composition provided by the present disclosure can comprise an immediate release pharmaceutical granulation comprising 4-((L-valyl)oxy) butanoic acid of, a modified release granulation comprising 4-((L-valyl)oxy) butanoic acid, and an excipient granulation.

A pharmaceutical composition provided by the present disclosure can comprise an immediate release pharmaceutical granulation comprising crystalline 4-((L-valyl)oxy) butanoic acid of, a modified release granulation comprising crystalline 4-((L-valyl)oxy) butanoic acid, and an excipient granulation.

A pharmaceutical composition can comprise, for example, from 1 g to 25 g of the 4-((L-valyl)oxy) butanoic acid.

A pharmaceutical composition can comprise, for example, from 2 wt % to 12 wt % of the excipient granulation; where wt % is based on the total weight of the pharmaceutical composition.

A pharmaceutical composition can comprise, for example, from 86 wt % to 98 wt % of the pharmaceutical granulation where wt % is based on the total weight of the pharmaceutical composition.

In a pharmaceutical composition, an immediate release pharmaceutical granulation can comprise from 26 wt % to 36 wt % of the 4-((L-valyl)oxy) butanoic acid; and a modified release pharmaceutical granulation comprises from 64 wt % to 74 wt % of the 4-((L-valyl)oxy) butanoic acid; and where wt % is based on the total weight of 4-((L-valyl)oxy) butanoic acid in the pharmaceutical composition.

In a pharmaceutical composition, a ratio of the weight of 4-((L-valyl)oxy) butanoic acid in the modified release pharmaceutical granulation to the weight of 4-((L-valyl)oxy) butanoic acid in the immediate release pharmaceutical granulation can be, for example, from 1.7 to 3.7.

A pharmaceutical composition can comprise, for example, from 15 wt % to 35 wt % wt % of the immediate release pharmaceutical granulation; from 55 wt % to 75 wt % of the modified release pharmaceutical granulation; and from 2 wt % to 12 wt % of the excipient granulation, wherein wt % is based on the total weight of the pharmaceutical composition.

A pharmaceutical composition can weigh, for example, from 2 g to 40 g.

Immediate release pharmaceutical granulations and modified release pharmaceutical granulations comprising 4-((L-valyl)oxy) butanoic acid or a pharmaceutically acceptable salt thereof are disclosed, for example, in U.S. Pat. No. 11,357,734 B2, U.S. Pat. No. 11,304,906 B2, U.S. Pat. No. 11,896,573 B2, U.S. Pat. No. 11,395,801 B2, and U.S. Pat. No. 11,510,892 B2, each of which is incorporated by reference in its entirety.

4-((L-Valyl)oxy) butanoic acid is a prodrug of γ-hydroxybutyric acid, which following oral administration, is absorbed by the gastrointestinal tract to provide 4-((L-valyl)oxy) butanoic acid in the systemic circulation, which is metabolized to provide γ-hydroxybutyric acid in the plasma of a patient. 4-((L-Valyl)oxy) butanoic acid has the structure of Formula (1):

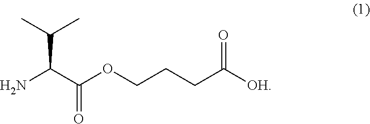

4-((L-Valyl)oxy) butanoic acid is metabolized to provide 0.512 equivalents of γ-hydroxybutyric acid. The valyl promoiety stabilizes γ-hydroxybutyric acid, which would otherwise cyclize.

4-((L-Valyl)oxy) butanoic acid can comprise crystalline 4-((L-valyl)oxy) butanoic acid as disclosed in U.S. Publication No. 2024/0092727 A1. For example crystalline 4-((L-valyl)oxy) butanoic acid can be characterized by an XRPD pattern comprising characteristic diffraction peaks at least at 8.28°±0.20°, 16.75°±0.20°, and 25.33±0.20° expressed as °2θ angles determined using Cu—Kα radiation; or an XRPD pattern comprising characteristic diffraction peaks at least at 8.28°±0.20°, 16.75°±0.20°, 17.64°±0.20°, 18.31°±0.20°, 19.42°±0.20°, 20.79°±0.20°, 25.33±0.20°, and 26.08°±0.20° expressed as °2θ angles determined using Cu—Kα radiation.

As used herein, 4-((L-valyl)oxy)butanoic acid includes crystalline 4-((L-valyl)oxy)butanoic acid and pharmaceutically acceptable salts of 4-((L-valyl)oxy)butanoic acid.

A pharmaceutical granulation can comprise from 1 g to 20 g of 4-((L-valyl)oxy)butanoic acid.

A pharmaceutical granulation can comprise an immediate release pharmaceutical granulation comprising a plurality of immediate release pharmaceutical granules comprising 4-((L-valyl)oxy)butanoic acid; and a modified release pharmaceutical granulation comprising a plurality of modified release pharmaceutical granules comprising 4-((L-valyl)oxy)butanoic acid.

A pharmaceutical granulation can comprise, for example, from 17 wt % to 37 wt % of an immediate release pharmaceutical granulation; and from 63 wt % to 83 wt % of a modified release pharmaceutical granulation, wherein wt % is based on the total weight of the pharmaceutical granulation.

In a pharmaceutical granulation, an immediate release pharmaceutical granulation can comprise, for example, from 2.5 g to 6.5 g of 4-((L-valyl)oxy)butanoic acid; and a modified release pharmaceutical granulation can comprise from 8 g to 12 g of 4-((L-valyl)oxy)butanoic acid.

An immediate release pharmaceutical granulation can comprise a plurality of immediate release pharmaceutical granules where the immediate release granules can be uncoated or can be coated with a seal coat.

An uncoated immediate release pharmaceutical granulation can comprise, for example, greater 80 wt % 4-((L-valyl)oxy)butanoic acid, where wt % is based on the total weight of the uncoated granulation.

For example, uncoated immediate release pharmaceutical granules can comprise greater 80 wt % 4-((L-valyl)oxy)butanoic acid, greater than 85 wt %, greater than 90 wt %, or greater than 95 wt % 4-((L-valyl)oxy)butanoic acid, where wt % is based on the total weight of the uncoated granules.

For example, uncoated immediate release pharmaceutical granules can comprise from 80 wt % to 98 wt % of 4-((L-valyl)oxy)butanoic acid, from 80 wt % to 95 wt %, from 85 wt % to 95 wt %, from 81 wt % to 97 wt %, from 82 wt % to 96 wt %, from 83 wt % to 95 wt %, from 84 wt % to 94 wt %, from 85 wt % to 93 wt %, or from 84 wt % to 92 wt % of 4-((L-valyl)oxy)butanoic acid, where wt % is based on the total weight of the uncoated granules.

An immediate release pharmaceutical granule can comprise, for example, a water-soluble polymer or a combination of water-soluble polymers.

Examples of suitable water-soluble polymers include hydroxypropyl cellulose, polyvinyl alcohol, hydroxypropylmethyl cellulose, hydroxypropylethyl cellulose, polyvinylpyrrolidone, polyethylene glycol, polyvinyl alcohol, povidone, crospovidone, and poloxamer.

A water-soluble polymer can comprise hydroxypropylmethyl cellulose.

An uncoated immediate release pharmaceutical granule can comprise, for example, from 1 wt % to 9 wt % of a water-soluble polymer, from 2 wt % to 8 wt %, from 3 wt % to 7 wt %, from 4 wt % to 6 wt %, or about 5 wt % of a water-soluble polymer such as hydroxypropylmethyl cellulose, where wt % is based on the total weight of the uncoated granule.

An uncoated immediate release pharmaceutical granule can comprise, for example, less than 10 wt % of a water-soluble polymer, less than 8 wt %, less than 6 wt %, less than 4 wt %, or less than 2 wt % of a water-soluble polymer, where wt % is based on the total weight of the uncoated granule.

An uncoated immediate release pharmaceutical granule of an immediate release granule can comprise, for example, an antistatic agent or a combination of antistatic agents.

Examples of suitable antistatic agents include talc (hydrous magnesium silicate), magnesium stearate, and silicon dioxide.

An antistatic agent can comprise hydrous magnesium silicate (talc).

An uncoated immediate release pharmaceutical granule can comprise, for example, from 1 wt % to 9 wt % of an antistatic agent, from 2 wt % to 8 wt %, from 3 wt % to 7 wt %, from 4 wt % to 6 wt %, or about 5 wt % of an antistatic agent such as hydrous magnesium silicate, where wt % is based on the total weight of the uncoated immediate release pharmaceutical granule.

An uncoated immediate release pharmaceutical granule can comprise, for example, from 85 wt % to 95 wt % of 4-((L-valyl)oxy)butanoic acid, from 1 wt % to 9 wt % of a water-soluble polymer such as hydroxypropylmethyl cellulose, and from 1 wt % to 9 wt % of an antistatic agent such as hydrous magnesium silicate, where wt % is based on the total weight of the uncoated immediate release pharmaceutical granule.

An uncoated immediate release pharmaceutical granule can comprise, for example, from 88 wt % to 92 wt % of 4-((L-valyl)oxy)butanoic acid, from 3 wt % to 7 wt % of a water-soluble polymer such as hydroxypropylmethyl cellulose, and from 3 wt % to 7 wt % of an antistatic agent such as hydrous magnesium silicate, where wt % is based on the total weight of the uncoated immediate release pharmaceutical granule.

An uncoated immediate release pharmaceutical granule can comprise, for example, from 89 wt % to 91 wt % of 4-((L-valyl)oxy)butanoic acid, from 4 wt % to 6 wt % of a water-soluble polymer such as hydroxypropylmethyl cellulose, and from 4 wt % to 6 wt % of an antistatic agent such as hydrous magnesium silicate, where wt % is based on the total weight of the uncoated immediate release pharmaceutical granule.

An uncoated immediate release pharmaceutical granule can comprise, for example, about 90 wt % of 4-((L-valyl)oxy)butanoic acid, about 5 wt % of a water-soluble polymer such as hydroxypropylmethyl cellulose, and about 5 wt % of an antistatic agent such as hydrous magnesium silicate, where wt % is based on the total weight of the uncoated immediate release pharmaceutical granule.

An uncoated immediate release pharmaceutical granule can have a homogeneous composition throughout the uncoated granule.

An uncoated immediate release pharmaceutical granulation can have an average granule size, for example, from 100 μm to 600 μm, from 150 μm to 550 μm, from 200 μm to 500 μm, from 250 μm to 450 μm or from 300 μm to 400 μm, where the average granule size is determined by sieve analysis or by laser diffraction.

An uncoated immediate release pharmaceutical granulation can have an average granule size, for example, greater than 100 μm, greater than 200, greater than 300, greater than 400 μm, or greater than 500 μm, where the average granule size is determined by sieve analysis or by laser diffraction.

An uncoated immediate release pharmaceutical granulation can have an average granule size, for example, of less than 600 μm, less than 500 μm, less than 400 μm, less than 300 μm, or less than 200 μm, where the average granule size is determined by sieve analysis or by laser diffraction.

An uncoated immediate release pharmaceutical granulation can have a bulk density, for example, greater than 0.40 g/mL, greater than 0.50 g/mL, greater than 0.60 g/mL, greater than 0.90 g/mL, greater than 1.10 g/mL, greater than 1.30 g/mL, or greater than 1.50 g/mL, wherein bulk density is determined using a bulk density cylinder.

A uncoated immediate release pharmaceutical granulation can have a bulk density, for example, from 0.40 g/mL to 1.60 g/mL, from 0.40 g/mL to 1.20 g/mL, from 0.40 g/mL to 0.80 g/mL, from 0.50 g/mL to 1.60 g/mL, from 0.50 g/mL to 1.40 g/mL, from 0.50 g/mL to 1.20 g/mL, from 0.60 g/mL to 1.60 g/mL, from 0.70 g/mL to 1.50 g/mL, from 0.80 g/mL to 1.40 g/mL, or from 1.00 g/mL to 1.20 g/mL, wherein bulk density is determined using a bulk density cylinder.

An uncoated immediate release pharmaceutical granulation can have a bulk density, for example, from 0.60 g/mL to 1.60 g/mL, from 0.70 g/mL to 1.50 g/mL, from 0.80 g/mL to 1.40 g/mL, or from 1.00 g/mL to 1.20 g/mL, wherein bulk density is determined using a bulk density cylinder.

An uncoated immediate release pharmaceutical granulation or granule can have a bulk density, for example, from 0.5 g/mL to 1.0 g/mL, from 0.5 g/mL to 0.9 g/mL, from 0.5 g/mL to 0.8 g/mL, from 0.5 g/mL to 0.7 g/mL, or from 0.6 g/mL to 0.7 g/mL. An uncoated pharmaceutical granulation or granule can have a bulk density, for example, greater than 0.5 g/mL, greater than 0.6 g/mL, greater than 0.7 g/mL, greater than 0.8 g/mL, or greater than 0.9 g/mL, wherein bulk density is determined using a bulk density cylinder.

An uncoated immediate release pharmaceutical granulation can comprise a plurality of granules characterized by a mode sphericity, for example, from 0.90 to 1, such as from 0.91 to 0.99, or from 0.92 to 0.98, where sphericity is determined using wet dispersion particle shape methods or by dynamic image analysis. An uncoated immediate release pharmaceutical granulation provided by the present disclosure can comprise a plurality of granules characterized by an average sphericity, for example, greater than 0.94, greater than 0.95, greater than 0.96, greater than 0.97, greater than 0.98, or greater than 0.99.

An uncoated immediate release pharmaceutical granulation provided by the present disclosure can be characterized by a friability value, for example, from 0% to 2%. Granules with low friability are easier to coat than are granules with high friability. Friability is defined as the amount of granules having a diameter less than 75 μm that are generated by subjecting a granulation to a sonic sifter operated at a vibration amplitude of 8 corresponding to 3,600 sonic energy pulses per minute for at least 2 minutes. An uncoated immediate release pharmaceutical granulation provided by the present disclosure can be characterized by a friability value, for example, of less than 2%, less than 1.8%, less than 1.6%, less than 1.4%, less than 1.2%, less than 1.0%, less than 0.8%, less than 0.6%, less than 0.4%, or less than 0.2%. An immediate release pharmaceutical granulation provided by the present disclosure can have a friability, for example, of 1.02% where friability is determined using a sonic sifter.

An uncoated immediate release pharmaceutical granulation can completely dissolve in water in less than 5 minutes following mixing and stirring in water, such as in less than 4 minutes, less than 3 minutes, less than 2 minutes, or in less than 1 minute. An immediate release pharmaceutical granulation can at least partially dissolve in water in less than 5 minutes following mixing and stirring in water, such as in less than 4 minutes, less than 3 minutes, less than 2 minutes, or in less than 1 minute.

An uncoated immediate release pharmaceutical granulation can be characterized by a dissolution profile in an aqueous buffer solution having a pH from 4.0 to 5.0 at a temperature from 20° C. to 25° C. such as 23° C., in which 80 wt % of the granulation is dissolved within 0.5 minutes, within 1 minute, within 2 minutes, within 3 minutes, within 4 minutes, or within 5 minutes.

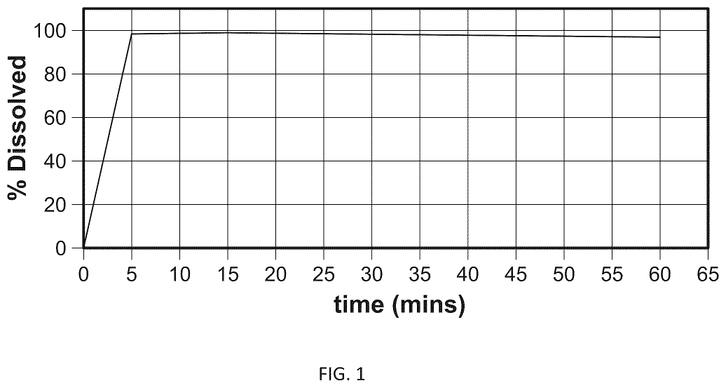

An example of a dissolution profile for an uncoated immediate release pharmaceutical granulation is shown in FIG. 1.

An immediate release pharmaceutical granule can be a coated with a seal coat.

A seal coating can minimize the ingress of moisture into the pharmaceutically active ingredient and thereby increase the storage stability of the coated microparticle by reducing hydrolysis of the pharmaceutically active ingredient. A seal coating can also minimize negative interactions between the modified release coating and the pharmaceutically active ingredient, and thereby increase the storage stability of the coated microparticle by reducing hydrolysis of 4-((L-valyl)oxy)butanoic acid.

A seal coating can comprise a water-soluble polymer such as, for example, hydroxypropylcellulose, polyvinyl alcohol, hydroxypropylmethyl cellulose, hydroxypropylethyl cellulose, polyvinylpyrrolidone, or polyethylene glycol.

A seal coating can have an average thickness, for example from 0.5 μm to 5 μm, from 1 μm to 4 μm, or from 1 μm to 3 μm. A seal coating can have an average thickness, for example, less than 5 μm, less than 4 μm, less than 3 μm, less than 2 μm, or less than 1 μm.

A coated immediate release pharmaceutical granulation may not be completely dissolved after mixing with water for longer than 1 minute, longer than 2 minutes, longer than 3 minutes, longer than 4 minutes, 5 minutes, or longer than 10 minutes.

Uncoated immediate release pharmaceutical granules have a dissolution profile as shown in FIG. 1. Uncoated immediate release pharmaceutical granules have a dissolution profile that is bioequivalent to the dissolution profile shown in FIG. 1.

A modified release granulation can comprise a plurality of modified release pharmaceutical granules. A modified release pharmaceutical granulation can comprise one or more modified release pharmaceutical granulations where the different granulations can have a different composition and/or property, such as for example, average granule size, active pharmaceutical ingredient, release profile, and/or excipient composition. Each of the two or more modified release pharmaceutical granulations can have a different release profile in the gastrointestinal tract. A modified release pharmaceutical granule can comprise an uncoated or coated immediate release pharmaceutical granule with a modified release coating surrounding the uncoated immediate release pharmaceutical granule. A modified release pharmaceutical granule can comprise an uncoated immediate release pharmaceutical granule with a modified release coating surrounding the uncoated immediate release pharmaceutical granule.

A modified release pharmaceutical granule can comprise, for example, from 70 wt % to 90 wt % of an immediate release pharmaceutical granule, from 76 wt % to 86 wt %, from 77 wt % to 85 wt %, from 78 wt % to 84 wt %, from 79 wt % to 83 wt %, from 70 wt % to 82 wt %, or 80.65 wt % of the immediate release core, where wt % is based on the total weight of the modified release pharmaceutical granule.

A modified release pharmaceutical granule can comprise, for example, greater than 65 wt % of an immediate release pharmaceutical granule, greater than 70 wt %, greater than 75 wt %, greater than 80 wt %, greater than 85 wt %, greater than 90 wt % or greater than 95 wt % of an immediate release pharmaceutical granule, where wt % is based on the total weight of the modified release pharmaceutical granule.

A modified release pharmaceutical granule can comprise, for example, 5 wt % to 40 wt %, of a modified release coating, from 10 wt % to 35 wt %, from 10 wt % to 30 wt %, from 14 wt % to 24 wt %, from 15 wt % to 23 wt %, from 16 wt % to 22 wt %, from 17 wt % to 21 wt %, from 18 wt % to 20 wt %, or about 19 wt % of a modified release coating, where wt % is based on the total weight of the modified release pharmaceutical granule.

A modified release pharmaceutical granule can comprise, for example, less than 30 wt % of a modified release coating, less than 25 wt %, less than 20 wt %, less than 15 wt %, less than 10 wt % or less than 5 wt % of a modified release coating, where wt % is based on the total weight of the modified release pharmaceutical granule.

A modified release pharmaceutical granule can comprise, for example, from 75 wt % to 85 wt % of an immediate release pharmaceutical granule and from 15 wt % to 25 wt % of a modified release coating, where wt % is based on the total weight of the modified release pharmaceutical granule.

A modified release pharmaceutical granule can comprise, for example, 77 wt % to 83 wt % of an immediate release pharmaceutical granule and from 17 wt % to 23 wt % of a modified release coating, where wt % is based on the total weight of the modified release pharmaceutical granule.

A modified release pharmaceutical granule can comprise, for example, from 65 wt % to 85 wt % of 4-((L-valyl)oxy)butanoic acid, from 68 wt % to 78 wt %, from 69 wt % to 77 wt %, from 70 wt % to 76 wt %, from 71 wt % to 75 wt %, from 72 wt % to 74 wt %, or about 73 wt % of 4-((L-valyl)oxy)butanoic acid, where wt % is based on the total weight of the modified release pharmaceutical granule.

A modified release coating can comprise a water-insoluble polymer, a water-soluble polymer, a lubricant, an antistatic agent, or a combination of any of the foregoing.

A modified release coating can comprise a water-insoluble polymer or a combination of water-insoluble polymers.

Examples of suitable water-insoluble polymers include ethylcellulose and polyvinyl acetates, polyacrylates, and polymethacrylates.

A water-insoluble polymer can comprise ethylcellulose.

A modified release coating can comprise, for example, from 68 wt % to 78 wt % of a water-insoluble polymer such as ethylcellulose, from 67 wt % to 77 wt %, from 68 wt % to 76 wt %, or from 69 wt % to 75 wt % of a water-insoluble polymer such as ethylcellulose, where wt % is based on the total weight of the modified release coating.

A modified release coating can comprise a water-soluble polymer or a combination of water-soluble polymers.

Examples of suitable water-soluble polymers include hydroxypropylcellulose, polyvinyl alcohol, hydroxypropylmethyl cellulose, hydroxypropylethyl cellulose, polyvinylpyrrolidone, or polyethylene glycol.

A water-soluble polymer can comprise hydroxypropylcellulose.

A modified release coating can comprise, for example, from 2 wt % to 6 wt % of a water-soluble polymer such as hydroxypropylcellulose, from 2.5 wt % to 5.5 wt %, from 3 wt % to 5 wt %, or from 3.5 wt % to 4.5 wt % of a water-soluble polymer such as hydroxypropylcellulose, where wt % is based on the total weight of the modified release coating.

A modified release coating can comprise a plasticizer or a combination of plasticizer s.

Examples of suitable plasticizers include dibutyl sebacate, polyethylene glycol, triacetin, and triethyl citrate.

A plasticizer can comprise dibutyl sebacate.

A modified release coating can comprise, for example, from 4 wt % to 12 wt % of a plasticizer such as dibutyl sebacate, from 5 wt % to 10 wt %, from 6 wt % to 9 wt %, or from 6 wt % to 8 wt % of a plasticizer such as dibutyl sebacate, where wt % is based on the total weight of the modified release coating.

A modified release coating can comprise an antistatic agent or a combination of antistatic agents.

Examples of suitable antistatic agents include talc (hydrous magnesium silicate), magnesium stearate, sodium stearyl fumarate, and silicon dioxide.

An antistatic agent can comprise hydrous magnesium silicate (talc).

A modified release coating can comprise, for example, from 10 wt % to 20 wt % of an antistatic agent such as hydrous magnesium silicate (talc), from 11 wt % to 19 wt %, from 12 wt % to 18 wt %, 13 wt % to 17 wt %, or from 14 wt % to 16 wt %, of an antistatic agent such as hydrous magnesium silicate (talc), where wt % is based on the total weight of the modified release coating.

A modified release coating can comprise, for example, a water-insoluble polymer; a water-soluble polymer; a lubricant; and an antistatic agent.

A modified release coating can comprise from 63 wt % to 83 wt % of a water-insoluble polymer; from 2 wt % to wt % of a water-soluble polymer; from 6 wt % to 10 wt % of a lubricant; and from 10 wt % to 20 wt % wt % of an antistatic agent; wherein wt % is based on the total weight of the modified release coating.

In a modified release pharmaceutical granule or a modified release coating, the water-insoluble polymer can comprise ethylcellulose; the water-soluble polymer can comprise hydroxypropylcellulose; the lubricant can comprise dibutyl sebacate; and the antistatic agent can comprise hydrous magnesium silicate.

A modified release pharmaceutical granule can comprise, for example, from 11 wt % to 17 wt % of a water insoluble polymer such as ethylcellulose, from 12 wt % to 16 wt %, from 13 wt % to 15 wt %, or 14.14 wt % of a water insoluble polymer such as ethylcellulose, where wt % is based on the total weight of the modified release pharmaceutical granule.

A modified release pharmaceutical granule can comprise, for example, from 0.45 wt % to 1.05 wt % of a water-soluble polymer such as hydroxypropylcellulose, from 0.55 wt % to 0.95 wt %, from 0.65 wt % to 0.85 wt %, from 0.70 to 0.80 wt % or 0.75 wt % of a water-soluble polymer such as hydroxypropylcellulose, where wt % is based on the total weight of the modified release pharmaceutical granule.

A modified release pharmaceutical granule can comprise, for example, from 0.5 wt % to 2.5 wt % of a plasticizer such as dibutyl sebacate, from 0.75 wt % to 2.25 wt %, from 1.0 wt % to 2.0 wt %, or from 1.25 wt % to 1.75 wt % of a plasticizer such as dibutyl sebacate, where wt % is based on the total weight of the modified release pharmaceutical granule.

A modified release pharmaceutical granule can comprise, for example, from 1.0 wt % to 5.0 wt % of an antistatic agent such as hydrous magnesium silicate (talc), from 1.5 wt % to 4.5 wt %, from 2.0 wt % to 4.0 wt %, 2.5 wt % to 3.5 wt %, or from 2.8 wt % to 3.2 wt %, of an antistatic agent such as hydrous magnesium silicate (talc), where wt % is based on the total weight of the modified release pharmaceutical granule.

A modified release pharmaceutical granule can comprise, for example, from 70 wt % to 90 wt % of an uncoated immediate release pharmaceutical granule, from 11 wt % to 17 wt % of a water insoluble polymer such as ethylcellulose, from 0.45 wt % to 1.05 wt % of a water-soluble polymer such as hydroxypropylcellulose; from 0.5 wt % to 2.5 wt % of a lubricant such as dibutyl sebacate; and from 1.0 wt % to 5.0 wt % of an antistatic agent such as hydrous magnesium silicate (talc), where wt % is based on the total weight of the modified release pharmaceutical granule.

A modified release coating can have a thickness, for example, from 5 μm to 40 μm, from 5 μm to 30 μm, from 5 μm to 20 μm, from 5 μm to 10 μm, from 10 μm to 40 μm, from 10 μm to 30 μm, or from 10 μm to 20 μm.

A modified release coating can have a thickness, for example, of less than 50 μm, less than 40 μm, less than 30, μm, less than 20 μm, or less than 10 μm.

A modified release pharmaceutical granule can comprise, for example, from 10 wt % to 45 wt % of the modified release coating, from 10 wt % to 40 wt %, or from 10 wt % to 30 wt %, where wt % is based on the total weight of the modified release pharmaceutical granule.

A modified release pharmaceutical granule can comprise, for example, of less than 45 wt %, less than 40 wt %, less than 35 wt %, less than 320 wt %, less than 25 wt %, less than 20 wt %, or less than 15 wt % of the modified release coating, where wt % is based on the total weight of the modified release pharmaceutical granule.

A modified release pharmaceutical granulation can have an average granule size, for example, from 100 μm to 600 μm, from 150 μm to 550 μm, from 200 μm to 500 μm, from 250 μm to 450 μm or from 300 μm to 400 μm, where the average granule size is determined by sieve analysis or laser diffraction.

A modified release pharmaceutical granulation can have an average granule size, for example, greater than 100 μm, greater than 200 μm, greater than 300 μm, greater than 400 μm, or greater than 500 μm, where the average granule size is determined by sieve analysis or laser diffraction.

A modified release pharmaceutical granulation can have an average granule size, for example, less than 600 μm, less than 550 μm, less than 500 μm, less than 450 μm, less, than 400 μm, less than 350 μm, less than 300 μm, or less than 250 μm, where the average granule size is determined by sieve analysis or laser diffraction.

A modified release pharmaceutical granulation provided by the present disclosure can have an average a bulk density from 0.4 g/mL to 1.60 g/mL, 0.40 g/mL to 1.40 g/mL, 0.50 g/mL to 0.90 g/mL, from 0.55 g/mL to 0.80 g/mL, from 0.60 g/mL to 75 g/mL, from 0.60 g/mL to 0.70 g/mL. A modified release pharmaceutical granulation can have a bulk density, for example, greater than 0.50 g/mL, greater than 0.60 g/mL, greater than 0.80 g/mL, greater than 0.90 g/mL, greater than 1.00 g/mL, greater than 1.20 g/mL, or greater than 1.40 g/mL, where bulk density is determined using a bulk density cylinder. A modified release pharmaceutical granulation can have a bulk density, for example, less than 1.6 g/mL, less than 1.20 g/mL, less than 1.00 g/mL, less than 0.80 g/mL, or less than 0.60 g/mL, where bulk density is determined using a bulk density cylinder.

A modified release pharmaceutical granulation can have a water content, for example, less than 2 wt %, less than 1.5 wt % less than 1 wt %, less than 0.5 wt % or less than 0.25 wt %, where wt % is based on the total weight of the modified release pharmaceutical granulation.

A modified release pharmaceutical granulation can have a water content, for example, from 0.1 wt % to 2 wt %, from 0.1 wt % to 1 wt %, or from 0.2 wt % to 0.5 wt %, where wt % is based on the total weight of the modified release pharmaceutical granulation.

A modified release pharmaceutical granulation can be configured to release the 4-((L-valyl)oxy)butanoic acid continuously with time in the gastrointestinal tract.

A modified release pharmaceutical granulation can have a dissolution profile in a buffered aqueous solution at a pH of 6.8 at a temperature of 23° C. characterized by from 20% to 40% dissolution at 1 hour, from 50% to 70% at 2 hours, from 60% to 80% at 3 hours, from 70% to 90% at 5 hours, and/or greater than 80% at 6 hours.

A modified release pharmaceutical granulation can have a dissolution profile in a buffered aqueous solution at a pH of 6.8 at a temperature of 23° C. characterized by from 25% to 35% dissolution at 1 hour, from 55% to 65% at 2 hours, from 65% to 75% at 3 hours, from 75% to 85% at 5 hours, and/or greater than 80% at 6 hours.

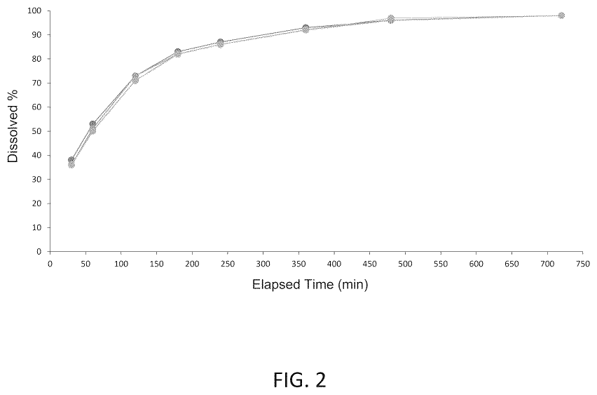

A modified release pharmaceutical granulation can have a dissolution profile in a buffered aqueous solution at a pH of 6.8 at a temperature of 23° C. as shown in FIG. 2.

A modified release pharmaceutical granulation can have a dissolution profile in a buffered aqueous solution at a pH of 6.8 at a temperature of 23° C. that is bioequivalent to the dissolution profile shown in FIG. 2.

A pharmaceutical granulation provided by the present disclosure can have an average bulk density, for example, from 0.6 g/mL to 0.9 g/mL, from 0.65 g/mL to 0.85 g/mL, or from 0.7 g/mL to 0.8 g/mL, where the average granule bulk density is determined using a bulk density cylinder.

A pharmaceutical granulation provided by the present disclosure can have an average tapped density that is, for example, within ±20% of the average tapped density of an excipient granulation with which the pharmaceutical granulation is combined, within ±15%, within ±10%, or within ±5% of the average tapped density of an excipient granulation with which the pharmaceutical granulation is combined, where average granule tapped density is determined according to USP <616>.

A pharmaceutical granulation provided by the present disclosure can have an average tapped density, for example, from 500 g/mL to 900 g/mL, from 550 g/L to 850 g/mL, from 600 g/mL to 800 g/mL, or from 650 g/mL to 750 g/mL, where the average granule tapped density is determined according to USP <616>.

A pharmaceutical granulation provided by the present disclosure can have a Hausner ratio that is, for example, within ±20% of the average Hausner ratio of an excipient granulation with which the pharmaceutical granulation is combined, within ±15%, within ±10%, or within ±5% of the Hausner ratio of an excipient with which the pharmaceutical granulation is combined, where the Hausner ratio is determined according to USP <1174>.

A pharmaceutical granulation provided by the present disclosure can have a Hausner ratio, for example, from 1.0 to 1.5, from 1.1 to 1.4, from 1.1 to 1.35, or from 1.1 to 1.25, where the Hausner ratio is determined according to USP <1174>.

A pharmaceutical granulation provided by the present disclosure can have a compressibility index that is, for example, within ±20% of the compressibility index of an excipient granulation with which the pharmaceutical granulation is combined, within ±15%, within ±10%, or within ±5% of the compressibility index of an excipient granulation with which the pharmaceutical granulation is combined.

A pharmaceutical granulation provided by the present disclosure can have a compressibility index, for example, from 8 to 22, from 10 to 20, or from 12 to 18.

A pharmaceutical granulation provided by the present disclosure can have a Flodex value that is, for example, within ±20% of the Flodex value of an excipient granulation with which the pharmaceutical granulation is combined, within +15%, within ±10%, or within ±5% of the Flodex value of an excipient granulation with which the pharmaceutical granulation is combined, where the Flodex value is determined according to USP <1174>.

A pharmaceutical granulation provided by the present disclosure can have a Flodex value, for example, of less than 5 mm, less than 4 mm, less than 3 mm, or less than 2 mm, where the Flodex value is determined according to USP <1174>.

A pharmaceutical granulation provided by the present disclosure can have a friability value, for example, of less than 0.2 such as about 0.1 where friability is determined using an ionic sifter.

A pharmaceutical composition provided by the present disclosure can comprise an excipient granulation or a combination of excipient granulations.

An excipient granulation provided by the present disclosure can improve the palatability of an orally administered pharmaceutical granulation.

Palatability can be defined as the overall appreciation of an oral medicinal product in relation to its smell, taste, aftertaste, and texture or feeling in the mouth. Palatability includes the overall acceptance of the taste, flavor, smell, does volume or size and texture of a medicine to be administered to the mouth and/or to be swallowed. An excipient granulation provided by the present disclosure can increase the viscosity of an orally administered solution comprising a pharmaceutical granulation and thereby reduce the coarse or rough feeling in the mouth.

To enhance the convenience during use, an excipient granulation can provide a viscous solution capable of suspending a pharmaceutical granulation within 30 seconds after being combined with water.

A suitable solution viscosity can be determined by a number of factors such as the density of the pharmaceutical granulation, the average size of the granules, the size distribution of the granules, and the average volume of the granules.

Excipient granules provided by the present disclosure can be particularly useful with high dose drugs. High dose drugs can require that large tablets or multiple tablets be administered. The inconvenience and/or discomfort of administering high dose drugs in the form of tablets can be avoided by using a pharmaceutical granulation. However, high dose granulations can produce a coarse or rough sensation in the mouth.

For convenience, it is desirable that a viscous solution prepared using an excipient granulation provided by the present disclosure be drinkable. A drinkable solution can have a viscosity, for example, from 50 cP to 500 cP at a temperature from 20° C. to 25° C., such as 23° C., determined using a No. 62 spindle at a speed of 12 rpm according to USP <912>, Method 1c. An excipient granulation provided by the present disclosure can be configured to provide a viscosity, when mixed with 40 mL of water at 23° C., for example, from 20 cP to 100 cP, determined using a No. 62 spindle at a speed of 12 rpm according to USP <912>, Method 1c.

However, excipient granules can also be used to provide higher viscosity pharmaceutical compositions that are more appropriately be eaten. For example, a pharmaceutical composition can have a viscosity from 100 cP to 500 cP at a temperature from 20° C. to 25° C., such as 23° C., determined using a No. 62 spindle at a speed of 12 rpm according to USP <912>, Method 1c.

Intended objectives of an excipient granulation provided by the present disclosure include, for example, (1) to provide a viscous solution having a viscosity from 10 cP to 200 cP such as from 40 cP to 80 cP, or from 50 cP to 70 cP, following mixing with an aqueous solution at 23° C. in less than 90 seconds such as in less than 30 seconds; (2) to provide an average excipient granule size similar to that of a pharmaceutical granulation; (3) to provide excipient granules that are physically robust to storage and transportation; and (4) to provide a homogeneous granule composition such that the excipient granules and other granules such as modified release pharmaceutical granules in the composition do not segregate and remain homogeneously dispersed throughout the suspension. It is desirable that the excipient granules and other granules in the composition remain homogeneously dispersed to facilitate the ability of the reconstituted solution and/or suspension to be homogeneous. Additional intended objectives can include (1) taste enhancement; (2) pH maintenance; (3) rapid dissolution, and (4) low wt % or low volume.

In an oral pharmaceutical composition provided by the present disclosure, the oral pharmaceutical composition can have a viscosity, for example, from 60 cP to 600 cP, such as from 200 cP to 600 cP, of from 300 cP to 600 cP, after 30 seconds following mixing with 40 mL of water at 23° C., determined using a No. 62 spindle at a speed of 12 rpm according to USP <912>, Method 1c. In an oral pharmaceutical composition provided by the present disclosure, the oral pharmaceutical composition can have a viscosity, for example, from 200 cP to 1,100 cP, such as from 400 cP to 1,000 cP, or from 600 cP to 1,000 cP, after 60 seconds following mixing with 40 mL of water at 23° C., determined using a No. 62 spindle at a speed of 12 rpm according to USP <912>, Method 1c An oral pharmaceutical composition can have a viscosity similar to the viscosity of a drinkable yogurt product.

In an oral pharmaceutical composition provided by the present disclosure, the oral pharmaceutical composition can have a viscosity, for example, from 60 cP to 600 cP, such as from 200 cP to 600 cP, of from 300 cP to 600 cP, from 15 seconds to 45 seconds following mixing with 40 mL of water at 23° C., determined using a No. 62 spindle at a speed of 12 rpm according to USP <912>, Method 1c. In an oral pharmaceutical composition provided by the present disclosure, the oral pharmaceutical composition can have a viscosity, for example, from 200 cP to 1,100 cP, such as from 400 cP to 1,000 cP, or from 600 cP to 1,000 cP, from 45 seconds to 75 seconds following mixing with 40 mL of water at 23° C., determined using a No. 62 spindle at a speed of 12 rpm according to USP <912>, Method 1c. An oral pharmaceutical composition can have a viscosity similar to the viscosity of a drinkable yogurt product.

In addition to the excipient granulation, the viscosity of an oral pharmaceutical formulation will be determined by the dissolution of the immediate release pharmaceutical granulation, and by the suspended modified release pharmaceutical granulation.

An excipient granulation provided by the present disclosure comprises a plurality of excipient granules. An excipient granulation provided by the present disclosure can comprise pharmaceutically acceptable excipients.

When combined with water, an excipient granulation provides a viscous aqueous solution. The viscosity of the viscous aqueous solution can be selected to improve the palatability of a pharmaceutical composition comprising a pharmaceutical granulation dispersed and suspended in the viscous solution. A suitable viscosity effective in improving the palatability of a pharmaceutical granulation can depend, for example, on the average size of the pharmaceutical granules, the size distribution of the pharmaceutical granules, the density of the pharmaceutical granules, the volume percent of the pharmaceutical granules in the viscous aqueous solution, or a combination of any of the foregoing. In general, it is desirable that the viscosity of an oral pharmaceutical composition for administration to a patient be drinkable. A drinkable composition can have a viscosity, for example, similar to that of a drinkable yogurt. For example, a drinkable composition can have viscosity from 50 cp to 600 cP, determined using a No. 62 spindle at a speed of 12 rpm according to USP <912>, Method 1c.

Excipient granules provided by the present disclosure can comprise a viscosifying agent, a disintegrant, and one or more additional excipients.

An excipient granule provided by the present disclosure can comprise an amount of each of the excipients suitable to accomplish an intended purpose of the excipient granulation such as, for example, to improve the palatability of a pharmaceutical granulation.

Excipient granules provided by the present disclosure can provide a viscous solution within 90 seconds, such as within 30 seconds, of being combined and mixed with an aqueous solution at a temperature from 20° C. to 25° C., such as 23° C. The maximum viscosity of the aqueous solution can be, for example, from 10 cP to 200 cP, from 25 CP to 150 cP, from 50 cP to 100 cP, from 40 cP to 80 cP, or from 50 cP to 70 cP at a temperature from 20° C. to 25° C., such as 23° C.

When combined with an aqueous solution an excipient granulation can provide a viscous solution comprising pharmaceutical granules dispersed and suspended in the viscous solution that are palatable for oral ingestion. The additional excipients of an excipient granulation provided by the present disclosure can be selected, for example, to reduce the sensation of granularity of the pharmaceutical granulation and to enhance the taste of the pharmaceutical composition. The additional excipients can be selected to adjust the pH of the pharmaceutical composition and to enhance the storage stability of the excipient granules and/or pharmaceutical granules.

An excipient granule provided by the present disclosure can comprise, for example, a viscosifying agent, a disintegrant, and one or more additional excipients. The one or more additional excipients is in addition to the viscosifying agent and the disintegrant.

An excipient granule provided by the present disclosure can comprise a viscosifying agent or a combination of viscosifying agents. A viscosifying agent can be a pharmaceutically acceptable viscosifying agent.

A viscosifying agent can be used to increase the viscosity of a liquid composition such as an aqueous composition including an oral pharmaceutical composition.

For example, excipient granules when mixed with water at a concentration of from 0.01 g/mL to 0.04 g/mL can provide a solution having a viscosity of greater than 20 cP, greater than 30 cP, greater than 40 cP, greater than 50 cP, greater than 60 cP, greater than 70 cP, greater than 80 cP, greater than 90 cP, or greater than 100 cP, in less than 2 minutes with stirring, agitating, and/or shaking at a temperature from 20° C. to 25° C. For example, excipient granules when mixed with water at a concentration of from 0.01 g/mL to 0.04 g/mL can provide a solution can provide a solution having a viscosity from 20 cP to 100 cP, such as from 40 cP to 80 cP in less than 30 seconds, in less than 60 seconds, in less than 90 seconds, or less than 120 seconds with stirring, agitating and/or shaking at a temperature from 20° C. to 25° C. such as 23° C., determined using a No. 62 spindle at a speed of 12 rpm according to USP <912>, Method 1c.

Suitable viscosifying agents include, for example, plant extracts such as a starch, a pectin, an alginate, carrageenan, acacia gum, tragacanth, and cellulose; modified polysaccharides such as modified starches, amidated pectin, propylene glycol alginate; cellulose ethers such as microcrystalline cellulose, hypromellose, hydroxypropyl cellulose, and carboxymethyl cellulose; animal extracts such as gelatin, chitosan, and caseinates; fermentation products such as xanthan gum, gellan gum, pullulan, and dextran; seed polysaccharides such as guar gum and locust bean extract; and a combination of any of the foregoing.

A viscosifying agent can comprise, for example, a hydrocolloid gum including plant-derived hydrocolloids such as pectin, agar, alginates, acacia, tragacanth, Karaya gum, guar gum, starches, cellulose and locust bean; hydrocolloid products of fermentation such as xanthan gum, dextran, gellan gum, and pullulan; semi-synthetic hydrocolloids including modified starches, cellulose ethers such as methylcellulose, hypromellose, ethylcellulose, hydroxypropylcellulose and hydroxyethylcellulose), amidated pectin and propylene glycol alginate; animal derived hydrocolloids such as gelatin, chitosan and caseinates; and a combination of any of the foregoing.

A viscosifying agent can include aluminum silicates, such as bentonite, hectorite, and magnesium aluminum silicate.

A viscosifying agent can comprise a carbomer.

A viscosifying agent can comprise a polyol, a sugar, an oligosaccharide such as sorbitol, liquid maltitol, sucrose, fructose, dextrose, maltodextrin, and polydextrose.

Other suitable viscosifying agents include polyethylene glycol, polyethylene oxide, colloidal silicon dioxide, silicones, glycerin, fatty acids and hard fats.

A viscosifying agent can include vinyl polymers such as water-soluble polyvinylpyrrolidones such as povidone and crospovidone having an average molecular weight from 2,500 g/mol to 1,250,000 g/mol; ethylene-vinyl acetates; a polyvinyl alcohol having for example a degree of hydrolysis greater than 88% and a molecular weight from 20,000 g/mole to 100,000 g/mol.

Suitable viscosifying agents include, for example, guar gum, xanthan gum, sodium carboxymethyl cellulose, and combinations of any of the foregoing.

A viscosifying agent can be in the form of particles, where the particles have an average particle size of less than 75 μm, less than 50 μm, or less than 25 μm. Particles of a viscosifying agent can have an average particle size, for example, from 10 μm to 75 μm, from 20 μm to 70 μm, or from 25 μm to 65 μm. A viscosifying agent such as xanthan gum can have an average particle size of less than 75 μm.

A viscosifying agent can comprise xanthan gum such as Xnatural® 75 available from CP Kelco.

A viscosifying agent such as xanthan gum can have a particle size, for example, wherein not less than 100% passes through an 80 mesh (180 μm) screen, such as not less than 92% through a 200 mesh (75 μm) screen as determined using screen sieving. A viscosifying agent such as xanthan gum can have a loss on drying (LOD), for example, of not more than 15%. A viscosifying agent such as xanthan gum can provide a viscosity, for example, of from 1,200 mPa×s to 1,600 mPa×s at 1 wt % of the viscosifying agent in a 1% KCL solution at 60 rpm as determined using transmittance. A viscosifying agent such as xanthan gum can provide a viscosity ratio, for example, of from 1.02 to 1.45. A viscosifying agent such as xanthan gum can provide a solution pH, for example, from 6.0 to 8.0 at 1 wt % of the viscosifying agent in distilled water as determined using transmittance.

An excipient granule can comprise, for example, from 1 wt % to 40 wt % of a viscosifying agent, from 1 wt % to 35 wt %, from 1 wt % to 30 wt %, from 1 wt % to 20 wt %, from 5 wt % to 15 wt %, or from 8 wt % to 12 wt % of a viscosifying agent, wherein wt % is based on the total weight of the excipient granule.

An excipient granule provided by the present disclosure can comprise a disintegrant or a combination of disintegrants. A disintegrant can be a pharmaceutically acceptable disintegrant.

A disintegrant facilitates rapid disintegration or breakdown and dissolution of the excipient granules following mixing with an aqueous solution at a temperature from 20° C. to 25° C.

It can be desirable that the excipient granules completely dissolve in water in less than 30 seconds, in less than 60 seconds, in less than 90 seconds, or within less than 120 seconds following mixing in an aqueous solution at a temperature from 20° C. to 25° C., such as 23° C.

A disintegrant can comprise, for example, crospovidone, croscarmellose sodium, low-substituted hydroxypropyl cellulose, sodium starch glycolate, chitosan hydrochloride, corn starch and pregelatinized starch, calcium alginate & calcium sodium alginate, docusate sodium, microcrystalline cellulose, hydroxypropyl starch, magnesium aluminum silicate, methylcellulose, sodium alginate, starch, pregelatinized starch, calcium carboxymethylcellulose, calcium cellulose glycolate, carmellose calcium, powdered cellulose, or a combination of any of the foregoing.

Examples of suitable disintegrants include crospovidone, croscarmellose sodium, sodium starch glycolate, soy polysaccharide, crosslinked alginic acid, gellan gum, xanthan gum, calcium silicate, and crosslinked polyacrylate.

An excipient can comprise crospovidone such as crospovidone CL-M available from BASF.

A suitable crospovidone disintegrating agent can have a pH value from 5 to 8 as a 1 wt % solution in water at a temperature from 20° C. to 25° C.; a bulk density from 0.25 g/cm³ to 0.45 g/cm³; and/or an average particle size from 20 μm to 40 μm.

An excipient granule can comprise, for example, from 1 wt % to 40 wt % of a disintegrant such as crospovidone, from 1 wt % to 30 wt %, from 1 wt % to 25 wt %, from 1 wt % to 20 wt %, from 5 wt % to 20 wt %, from 7 wt % to 17 wt %, or from 10 wt % to 14 wt % of a disintegrant such as crospovidone, wherein wt % is based on the total weight of the excipient granule.

An excipient granule provided by the present disclosure can comprise one or more additional excipients. An additional excipient can be a pharmaceutically acceptable excipient.

Examples of suitable excipients include preservatives, antioxidants, adsorbents, acidifying agents, alkalinizing agents, buffering agents, chelating agents, colorants, clarifying agents, emulsifying agents, flavorings, water-soluble polymers, diluents, binders, lubricants, wetting agents, surfactants, glidants, and filler.

The one or more additional excipients can comprise a diluent, a binder, a flavoring agent, a buffer, an acid, a sweetening agent, a filler, or a combination of any of the foregoing.

An excipient granule provided by the present disclosure can comprise, for example, from 40 wt % to 98 wt % of an additional excipient or combination of additional excipients, not including the viscosifying agent and the dispersant. An excipient granule provided by the present disclosure can comprise, for example, from 40 wt % to 98 wt % of one or more additional excipients, from 50 wt % to 95 wt %, from 60 wt % to 90 wt %, from 70 wt % to 85 wt %, or from 75 wt % to 85 wt % of one or more additional excipients, where wt % is based on the total weight of the excipient granule.

An excipient granule provided by the present disclosure can comprise, for example, less than 95 wt % of an additional excipient, less than 90 wt %, less than 80 wt %, less than 70 wt %, or less than 60 wt % of one or more additional excipients, where wt % is based on the total weight of the excipient granule. An excipient granule provided by the present disclosure can comprise, for example, greater than 40 wt % of one or more additional excipients, greater than 50 wt %, greater than 60 wt %, greater than 70 wt %, greater than 80 wt %, or greater than 90 wt % of one or more additional excipients, where wt % is based on the total weight of the excipient granule.

An excipient granule can comprise a diluent or combination of diluents. A diluent can be a pharmaceutically acceptable diluent.

A diluent can provide bulk to the excipient granule, enhance flow properties, and facilitate processing.

Examples of suitable diluents include microcrystalline cellulose, powdered cellulose, anhydrous lactose, lactose monohydrate, spray-dried lactose, mannitol, starch, pregelatinized starch, maize starch, corn starch, sorbitol, sucrose, compressible sugar, confectioner's sugar, sugar spheres, dextrates, dextrin, dextrose, dibasic calcium phosphate, anhydrous calcium phosphate, calcium carbonate, maltose, maltodextrin, kaolin, dibasic calcium phosphate, dihydrate calcium phosphate, tribasic calcium phosphate, calcium sulfate, cellaburate, calcium lactate, cellulose acetate, silicified microcrystalline cellulose, cellulose acetate, corn syrup, pregelatinized starch and corn starch, corn syrup solids, erythritol, ethylcellulose, ethyl acrylate and methyl methacrylate copolymer dispersion, fructose, isomalt, alpha-lactalbumin, lactitol, magnesium carbonate, magnesium oxide, methacrylic acid and ethyl acrylate copolymer, methacrylic acid and methyl methacrylate copolymer, polydextrose, sodium chloride, simethicone, pregelatinized modified starch, pea starch, hydroxypropyl pea starch, pregelatinized hydroxypropyl pea starch, potato starch, hydroxypropyl potato starch, pregelatinized hydroxypropyl potato starch, starch, tapioca, wheat starch, hydrogenated starch hydrolysate, pullulan, talc, amino methacrylate copolymer, trehalose, xylitol, and a combination of any of the foregoing.

Examples of suitable diluents include lactose, microcrystalline cellulose, starch, calcium phosphate such as anhydrous dibasic calcium phosphate, hydrated dibasic calcium phosphate, and tribasic calcium phosphate, calcium carbonate, maltodextrin, mannitol, sorbitol, and sodium chloride.

A diluent can comprise anhydrous dibasic calcium phosphate.

An anhydrous dibasic calcium phosphate can be A-Comprez® available from Markan Global Enterprises.

An excipient granule can comprise, for example, from 1 wt % to 40 wt %, from 10 wt % to 30 wt %, from 15 wt % to 25 wt %, or from 17 wt % to 23 wt % of a diluent such as anhydrous dibasic calcium phosphate, where wt % is based on the total weight of the excipient granule.

An excipient granule provided by the present disclosure can comprise a binder or a combination of binders. A binder can be a pharmaceutically acceptable binder.

A binder promotes cohesion of the of the constituents of the excipient granule.

Examples of suitable binders include natural binders such as starch, pregelatinized starchy, sodium alginate, and gelatin; synthetic and semi-synthetic binders such as polyvinylpyrrolidone, methylcellulose, hydroxypropyl methyl cellulose, polymethacrylates, sodium carboxy methyl cellulose, polyethylene glycol, and methylcellulose; saccharides such as lactose, sucrose, starches, cellulose and modified cellulose such as microcrystalline cellulose and cellulose ethers such as hydroxypropyl cellulose, and sugar derivatives such as sorbitol, xylitol, and mannitol.

Other examples of suitable binders include acacia, copovidone, carbomer, corn starch and pregelatinized starch, calcium carboxymethylcellulose, calcium cellulose glycolate, carmellosum calcium, carboxymethylcellulose sodium, carmellose sodium, Ceratonia, chitosan hydrochloride, dextrates, dextrin, ethylcellulose, liquid glucose, guar gum, hydroxyethyl cellulose, hydroxyethylmethyl cellulose, hydroxypropyl cellulose, low-substituted hydroxypropyl cellulose, hydroxypropyl starch, hydroxypropyl methylcellulose, inulin, magnesium aluminum silicate, maltodextrin, methylcellulose, polyethylene glycol, polyethylene oxide, povidone, sodium alginate, starch, pregelatinized starch, sucrose, compressible sugar, zein, gelatin, polymethylmethacrylates, sorbitol, glucose, and sodium alginate.

Examples of suitable binders include hydroxypropyl cellulose, polyvinyl alcohol, hydroxypropylmethyl cellulose, hydroxypropylethyl cellulose, polyvinylpyrrolidone, or polyethylene glycol.

A binder can comprise a polyvinylpyrrolidone.

A suitable polyvinylpyrrolidone can be characterized by an average molecular weight, for example, of 58,000 Daltons, wherein the average molecular weight is determined by column chromatography. A suitable polyvinylpyrrolidone can have a viscosity, for example, of 2.5 cP in a 5% aqueous solution at 25° C. A suitable polyvinylpyrrolidone can have an average particle size, for example, of 80 μm. A suitable polyvinylpyrrolidone can have a bulk density, for example, of 0.36 g/cm³ and/or a tapped density of 0.47 g/cm³.

An excipient granule can comprise, for example, from 1 wt % to 30 wt %, from 2 wt % to 25 wt % from 5 wt % to 20 wt %, or from 7 wt % to 13 wt % of a binder such as polyvinylpyrrolidone, where wt % is based on the total weight of the excipient granule.

An excipient granule provided by the present disclosure can comprise a flavoring agent or a combination of flavoring agents. A flavoring agent can be a pharmaceutically acceptable flavoring agent.

A flavoring agent can render a pharmaceutical composition more palatable and can mask an unpleasant taste of an active pharmaceutical ingredient. The nature of the unpleasant taste to be masked can determine the selection of a suitable flavoring agent.

Examples of suitable flavoring agents include vanillin, peppermint flavor powder, berry flavor powder, strawberry flavor powder, orange flavor powder, lemon flavor powder, orange essence, ethyl maltol, eucalyptus oil, isobutyl alcohol, sodium succinate, adipic acid, almond oil, anethole, benzaldehyde, denatonium benzoate, ethyl acetate, ethyl vanillin, ethylcellulose, fructose, fumaric acid, 1-glutamic acid, hydrochloride, lactitol, leucine, malic acid, maltol, menthol/racementhol, methionine, methyl salicylate, monosodium glutamate, peppermint oil, strawberry flavor, liquid, peppermint spirit, racemethionine, rose oil, rose water, stronger, sodium acetate, sodium lactate solution, tartaric acid, thymol, fumaric acid, inulin, isomalt, and neohesperidin dihydrochalcone.

A flavoring agent can comprise a citric fruit flavoring agent, a berry flavoring agent, a punch flavoring agent, or a combination of any of the foregoing.

An excipient granule provided by the present disclosure can comprise, for example, from 1 wt % to 40 wt % of a flavoring agent, from 5 wt % to 35 wt %, from 10 wt % to 35 wt % from 15 wt % to 35 wt %, from 20 wt % to 30 wt %, or from 22 wt % to 28 wt %, of a flavoring agent, where wt % is based on the total weight of the excipient granule.

An excipient granule provided by the present disclosure can comprise a buffer or a combination of buffers. A buffer can be a pharmaceutically acceptable buffer.

Examples of suitable buffers include sodium citrate dihydrate, citric acid anhydrous, sodium phosphate dibasic, and sodium phosphate monobasic, potassium metaphosphate, potassium phosphate, monobasic sodium acetate, and anhydrous sodium citrate.

A buffer can comprise sodium citrate dihydrate.

An excipient granule can comprise, for example, from 0.1 wt % to 10 wt % of a buffer, from 0.5 wt % to 8 wt %, from 0.5 wt % to 6.5 wt %, from 1 wt % to 5 wt %, or from 2 wt % to 4 wt % of a buffer such as sodium citrate dihydrate, where wt % is based on the total weight of the excipient granule.

An excipient granule provided by the present disclosure can comprise an acidifying agent or a combination of acidifying agents. An acidifying agent can be a pharmaceutically acceptable acidifying agent.

Examples of suitable acidifying agent include anhydrous sodium citrate, fumaric acid, hydrochloric acid, and nitric acid.

An excipient granule can comprise, for example, from 0.1 wt % to 10 wt % of an acid, from 0.5 wt % to 8 wt %, from 1 wt % to 7 wt %, from 2 wt % to 6 wt %, or from 3 wt % to 5 wt % of an acidifying agent such as anhydrous sodium citrate, where wt % is based on the total weight of the excipient granule.

An excipient granule provided by the present disclosure can comprise a sweetening agent or a combination of sweetening agents. A sweetening agent can comprise a pharmaceutically acceptable sweetening agent.

Examples of suitable sweetening agents include sucralose, saccharin sodium, neotame, sucrose, acesulfane potassium, aspartame, aspartame acesulfane, corn syrup, corn syrup solids, dextrates, dextrose, dextrose excipient, erythritol, fructose, galactose, glucose, glycerin, inulin, invert sugar, isomalt, lactitol, maltitol, maltose, mannitol, saccharin, saccharin calcium, sorbitol, starch hydrolysate, hydrogenated, sugar, compressible, sugar, confectioner's, tagatose, trehalose, and xylitol.

Examples of suitable sweetening agents include mannitol, sucralose, aspartame, dextrose, glycerin, saccharin sodium, sorbitol, and sucrose.

A sweetening agent can comprise sucralose.

An excipient granule can comprise, for example, from 0.1 wt % to 10 wt % of a sweetening agent such as sucralose, from 0.1 wt % to 7 wt %, from 0.1 wt % to 6 wt %, from 1 wt % to 5 wt %, or from 2 wt % to 4 wt % of a sweetening agent such as sucralose, where wt % is based on the total weight of the excipient granule.

An excipient granule provided by the present disclosure can comprise a filler or combination of filler. A filler can be a pharmaceutically acceptable filler.

Examples of suitable filler include starch 1500, starch 1500 (gelatinized), microcrystalline cellulose, calcium phosphate powder, dicalcium phosphate dihydrate, mannitol, or dicalcium phosphate powder, lactose, calcium carbonate, calcium sulfate, compressible sugars, dextrates, dextrin, dextrose, kaolin, magnesium carbonate, magnesium oxide, maltodextrin, powdered cellulose, sucrose, and combinations of any of the foregoing.

A filler can comprise microcrystalline cellulose such as Avicel® PH-101 or Celphere® CP-305 available from Asahi-Kasei.

An excipient granule provided by the present disclosure can comprise, for example, from 1 wt % to 20 wt % of a filler such as microcrystalline cellulose, from 5 wt % to 15 wt %, or from 7 wt % to 13 wt % of a filler such as microcrystalline cellulose, where wt % is based on the total weight of the excipient granule.

An excipient granule provided by the present disclosure can comprise, for example, a viscosifying agent, a disintegrant, and one or more additional excipients where the one or more additional excipients can comprise a filler or combination of fillers. An excipient granule can comprise, for example, from 40 wt % to 98 wt % of a filler, from 50 wt % to 90 wt %, or from 60 wt % to 80 wt % of a filler, where wt % is based on the total weight of the excipient granule. An excipient granule can comprise, for example, less than 90 wt % of a filler, less than 80 wt %, less than 70 wt %, less than 60 wt %, less than 50 wt % of a filler, where wt % is based on the total weight of the excipient granule. An excipient granule can comprise, for example greater than 40 wt % of a filler, greater than 50 wt %, greater than 60 wt %, greater than 70 wt %, greater than 80 wt %, or greater than 90 wt % of a filler, where wt % is based on the total weight of the excipient granules.

An excipient granule provided by the present disclosure can comprise a pharmaceutically acceptable glidant or combination of pharmaceutically acceptable glidants. Examples of suitable pharmaceutically acceptable glidants include magnesium stearate, magnesium silicate, calcium stearate, sodium lauryl sulphate, sodium stearyl fumarate, magnesium lauryl sulphate, stearic acid, calcium stearate, glyceryl behenate, behenoyl polyoxylglycerides, glyceryl dibehenate, lauric acid, glyceryl monostearate, glyceryl tristearate, myristic acid, palmitic acid, poloxamer, polyethylene glycol, polyethylene glycol 3350, polysorbate 20, polyoxyl 10 oleyl ether, polyoxyl 15 hydroxystearate, polysorbate 40, polyoxyl 20 cetostearyl ether, polyoxyl 40 stearate, polysorbate 60, polysorbate 80, potassium benzoate, sodium benzoate, sorbitan monolaurate, sorbitan monooleate, sodium stearate, sorbitan monopalmitate, sorbitan monostearate, zinc stearate, sorbitan sesquioleate, sorbitan trioleate, and talc.

An excipient granule provided by the present disclosure can comprise a pharmaceutically acceptable lubricant or combination of pharmaceutically acceptable lubricants. Examples of suitable pharmaceutically acceptable lubricants include colloidal silicon dioxide, talc, tribasic calcium phosphate, calcium silicate, cellulose, powdered, magnesium oxide, sodium stearate, magnesium silicate, silica, dental-type, magnesium trisilicate, and hydrophobic colloidal silica.

An excipient granule provided by the present disclosure can comprise a pharmaceutically acceptable alkalinizing agent or combination of pharmaceutically acceptable alkalinizing agents. Examples of suitable pharmaceutically acceptable alkalinizing agents include ammonium carbonate, diethanolamine, monoethaolamine, potassium hydroxide, sodium bicarbonate, sodium borate, sodium carbonate, sodium hydroxide, and trolamine.

An excipient granule provided by the present disclosure can comprise a pharmaceutically acceptable surfactant or combination of pharmaceutically acceptable surfactants. Examples of suitable pharmaceutically acceptable surfactants include behenoyl polyoxylglycerides, Polysorbate 20, Polysorbate 40, docusate sodium, Polysorbate 60, Polysorbate 80, benzalkonium chloride, caprylocaproyl polyoxylglycerides, cetylpyridinium chloride, lauroyl polyoxylglycerides, linoleoyl polyoxylglycerides, Octoxynol 9, oleoyl polyoxylglycerides, poloxamer, Polyoxyl 10 oleyl ether, Polyoxyl 15 hydroxystearate, Nonoxynol 9, Polyoxyl 20 cetostearyl ether, Polyoxyl 40 stearate, pullulan, polyoxyl lauryl ether, polyoxyl stearyl ether, sodium lauryl sulfate, sorbitan monolaurate, sorbitan monooleate, polyoxyl stearate, sorbitan monopalmitate, sorbitan monostearate, stearoyl polyoxylglycerides, sorbitan sesquioleate, sorbitan trioleate, and tyloxapol.

An excipient granule provided by the present disclosure can comprise a pharmaceutically acceptable coloring agent or combination of pharmaceutically acceptable coloring agents. Examples of suitable pharmaceutically acceptable coloring agents include caramel, ferric oxide, titanium dioxide, ferric oxide, aluminum oxide, FD & C Red #40/Allura Red AC, amaranth, FD & C Blue #1/Brilliant Blue FCF, canthaxanthin, carmine, carmoisine (Azorubine), curcumin (tumeric), FD & C Red #3/Erythrosine, Fast Green FCF, Green S (Lissamine Green), D & C Red #30/Helendon Pink, FD & C Blue #2/Indigo Carmine, iron oxide black, iron oxide red, D & C Red #7/Lithol Rubin BK, Patent Blue V. D & C Red #28/Phloxine B, Iron Oxide Yellow, D & C Red #27/Phloxine O, Ponceau 4R (Cochineal Red A), Quinoline Yellow WS, D & C Yellow #10, Riboflavin (Lactoflavin), FD & C Yellow #5/Tartrazine, and FD & C Yellow #6/Sunset Yellow FCF.

An excipient granule and excipient granulation provided by the present disclosure can comprise, for example, from 2 wt % to 22 wt % of a binder; from 10 wt % to 30 wt % of a diluent; from 1 wt % to 20 wt % of a filler; from 15 wt % to 35 wt % of a flavoring agent; from 0.5 wt % to 6.5 wt % of a buffer; from 0.5 wt % to 8 wt % of an acidifying agent; and/or from 0.1 wt % to 6 wt % of a sweetening agent, wherein wt % is based on the total weight of the excipient granule or excipient granulation.

An excipient granule and excipient granulation provided by the present disclosure can comprise, for example, from 1 wt % to 35 wt % of a viscosifying agent; and from 1 wt % to 25 wt % of a disintegrant; wherein wt % is based on the total weight of the excipient granule or excipient granulation.

An excipient granule and excipient granulation provided by the present disclosure can comprise, for example, from 5 wt % to 15 wt % of a binder; from 17 wt % to 27 wt % of a diluent; from 6 wt % to 16 wt % of a filler; from 20 wt % to 30 wt % of a flavoring agent; from 1 wt % to 5 wt % of a buffer; from 2 wt % to 6 wt % of an acidifying agent; and/or from 1 wt % to 5 wt % of a sweetening agent, where wt % is based on the total weight of the excipient granule or excipient granulation.

An excipient granule and excipient granulation provided by the present disclosure can comprise, for example, from 5 wt % to 15 wt % of a viscosifying agent; and from 7 wt % to 17 wt % of a disintegrant; where wt % is based on the total weight of the excipient granules or excipient granulation.

In an excipient granule and excipient granulation provided by the present disclosure the viscosifying agent can comprise xanthan gum; the disintegrant can comprise crospovidone; the binder can comprise polyvinylpyrrolidone; the diluent comprise can dicalcium phosphate; the filler can comprise microcrystalline cellulose; the flavoring agent can comprise a berry/punch flavoring agent; the buffer can comprise sodium citrate dihydrate; the acidifying agent can comprise citric acid anhydrous; and/or the sweetening agent can comprise sucralose, where wt % is based on the total weight of the excipient granule or excipient granulation.

An excipient granulation provided by the present disclosure can have an average granule size, for example, from 100 μm to 600 μm, from 200 μm to 600 μm, from 100 μm to 550 μm, from 100 μm to 500 μm, from 200 μm to 500 μm or from 250 μm to 450 μm, where the average granule size is determined by sieve analysis or laser diffraction.

An excipient granulation can have a particle size distribution characterized, for example, by 8.5 wt % greater than 35 mesh (500 μm), 39 wt % from 35-60 mesh (500-250 μm), 52.2% pan, less than 35 mesh (500 μm) 52.2 wt %; and the particle size is determined by sieve analysis or laser diffraction.

An excipient granulation (35-60 mesh fraction) can have an average bulk density, for example, from 0.5 g/mL to 0.9 g/mL, where the bulk density is determined using a bulk density cylinder.

An excipient granulation provided by the present disclosure can have a bulk density, for example, from 0.5 g/mL to 0.7 g/mL, from 0.6 g/mL to 0.9 g/mL, from 0.65 g/L to 0.85 g/mL, or from 0.7 g/mL to 0.8 g/mL, where the bulk density is determined according to USP <616>, Method 1.

An excipient granulation provided by the present disclosure can have an average tapped density, for example, from 500 g/mL to 900 g/mL, from 550 g/L to 850 g/mL, from 600 g/mL to 800 g/mL, or from 650 g/mL to 750 g/m, where the average tapped density is determined according to USP <616>.

An excipient granulation provided by the present disclosure can have a Hausner ratio, for example, from 1.0 to 1.5, from 1.1 to 1.4, from 1.1 to 1.35, or from 1.1 to 1.25, where the pharmaceutical granulation has a comparable Hausner ratio, where the average Hausner ratio is determined according to USP <1174>.

An excipient granulation provided by the present disclosure can have a compressibility index, for example, from 8 to 22, from 10 to 20, or from 12 to 18.

An excipient granulation provided by the present disclosure can have a Flodex value, for example, of less than 5 mm, less than 4 mm, less than 3 mm, or less than 2 mm, where the Flodex value is determined according to USP <1174>.

An excipient granulation provided by the present disclosure can have a friability, for example, of less than 2, or less than 1, where the friability is determined using a sonic sifter.

An excipient granulation provided by the present disclosure can have a loss on drying, for example, from 1 wt % to 4 wt %, such as from 1.5 wt % to 3.5 wt %, or from 2 wt % to 3 wt %, where the loss on drying is determined by thermogravimetric analysis.

An excipient granulation provided by the present disclosure can completely dissolve when mixed with an aqueous solution at a temperature of 20° C. to 25° C., for example, in less than 120 seconds, in less than 90 seconds, in less than 60 seconds, or less than 30 seconds, where completely dissolved is defined as the time from when the excipient granulation is combined with the aqueous solution to the time when the viscosity of the viscous solution is greater than 90% of the maximum viscosity.

A solution prepared by combining from 1 wt % to 5 wt % of an excipient granulation with from 95 wt % to 99 wt % water at a temperature from 20° C. to 25° C. can achieve a maximum viscosity, for example, within from 15 seconds to 120 seconds such as from 15 seconds to 60 seconds following mixing, where wt % is based on the total weight of the excipient granulation and the water. Mixing can be accomplished, for example, by stirring, agitating, and/or swirling the excipient granulation in water or a non-viscous aqueous solution.

A solution prepared by combining from 1 wt % to 5 wt % of the excipient granulation with from 95 wt % to 99 wt % water at a temperature from 20° C. to 25° C., such as 23° C., can provide a solution having a viscosity, for example, of from 25 cp to 200 cp.

An excipient granulation provided by the present disclosure, when reconstituted as a 1 w/v % solution in water at a temperature from 20° C. to 25° C., such as 23° C., can completely dissolve in less than 60 seconds or in less than 30 seconds.

An excipient granulation provided by the present disclosure, when reconstituted as a 1 w/v % solution in water at a temperature from 20° C. to 25° C., such as 23° C., can provide a solution having a pH, for example from 4.0 to 8.0, such as from 4.0 to 7.0, or from 4.0 to 5.0.

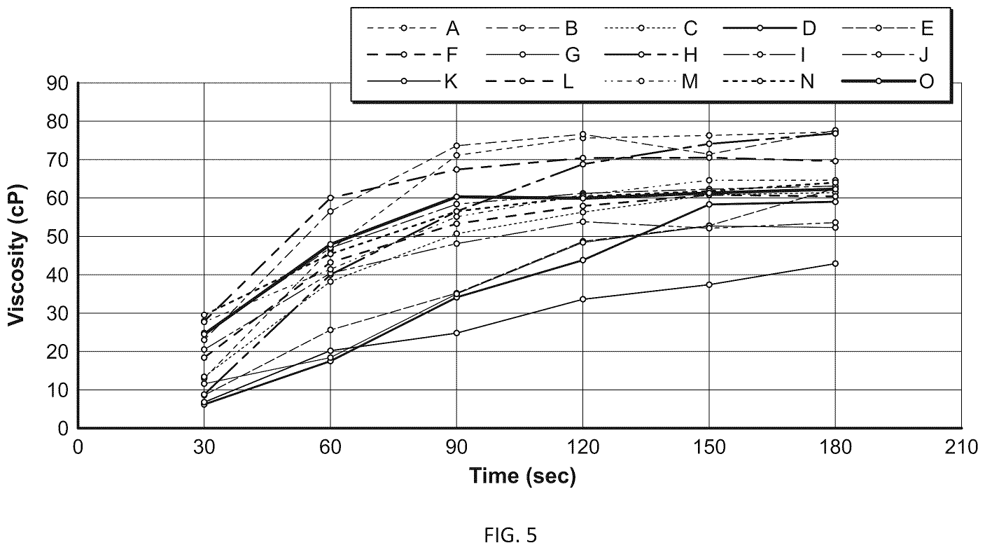

When 1.2 g of an excipient granulation provided by the present disclosure is reconstituted in 40 mL of water at a temperature from 20° C. to 25° C., such as 23° C., the resulting solution exhibits a viscosity vs. time profile as shown in FIG. 5.

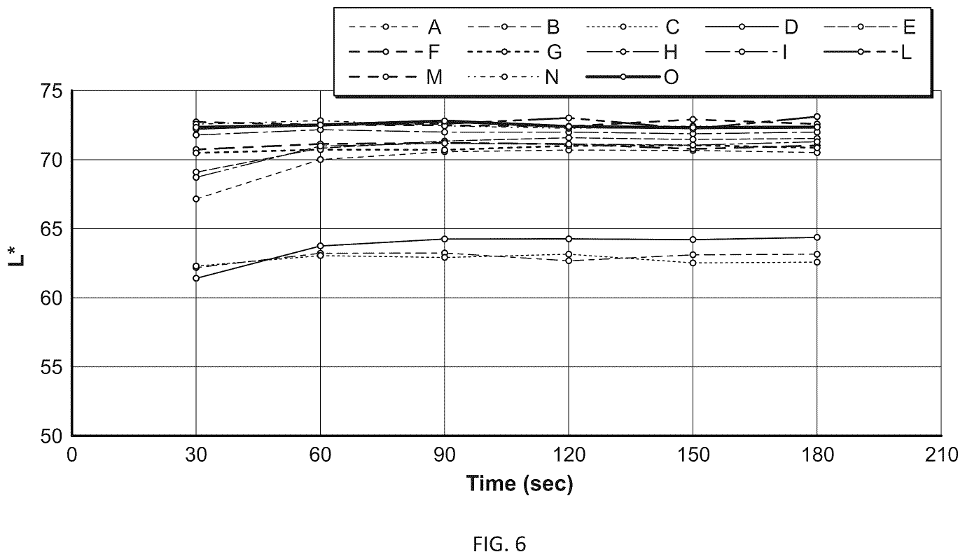

When 1.2 g of an excipient granulation provided by the present disclosure is reconstituted in 40 mL of water at a temperature from 20° C. to 25° C., such as 23° C., the resulting solution exhibits a luminosity vs. time profile as shown in FIG. 6.

An excipient granulation can be configured to provide a viscosity of from 20 cP to 100 cP when 1.2 gm of the excipient granulation is combined with 40 mL of water at a temperature from 20° C. to 25° C., such as 23° C.

After mixing a solution of 1.2 g of an excipient granulation provided by the present disclosure and 40 mL of water at a temperature from 20° C. to 25° C., such as 23° C., for 90 seconds, the solution can have a viscosity within a range from 45 cP to 80 cP.

After mixing a solution of 1.2 g of an excipient granulation provided by the present disclosure and 40 mL of water at a temperature from 20° C. to 25° C., such as 23° C., for 60 seconds, the solution can have a viscosity within a range from 35 cP to 60 cP.

After mixing a solution of 1.2 g of an excipient granulation provided by the present disclosure and 40 mL of water at a temperature from 20° C. to 25° C., such as 23° C., for 30 seconds, the solution can have a viscosity within a range from 15 cP to 40 cP.

An excipient granulation provided by the present disclosure can provide a solution having a reconstitution pH of from 4 to 6.

Physical properties of an excipient granulation can be selected to minimize or prevent settling or segregation of a pharmaceutical composition comprising an excipient granulation and a pharmaceutical granulation. To facilitate the ability of the pharmaceutical granulation to become homogeneously dispersed in a viscous solution prepared using the excipient granulation, it can be desirable that the pharmaceutical composition comprising the excipient granulation and the pharmaceutical granulation be homogenously dispersed and remain homogenously dispersed during storage and transportation. Homogeneous dispersion of an excipient granulation and a pharmaceutical granulation of a pharmaceutical composition can be facilitated by having the physical properties of the excipient granulation and a pharmaceutical granulation be similar. For example, it can be desirable that certain physical properties such as the average particle size, the bulk density, the tapped density, the Hausner ratio, the compressibility index, the Flodex value, and/or the friability value of the excipient granulation and the pharmaceutical granulation be similar. For example, it can be desirable that a value of a physical property of an excipient granulation be within +/−20%, within +/−15%, within +/−10%, within +/−5%, or within less than +/−2% the value of the corresponding property of a pharmaceutical granulation.

For a pharmaceutical granulation comprising more than one population of granulations such as an immediate release pharmaceutical granulation and a modified release pharmaceutical granulation it can be desirable that a value of a physical property of an excipient granulation be within +/−20%, within +/−15%, within +/−10%, within +/−5%, or within less than +/−2% the value of the corresponding property of each of the immediate release pharmaceutical granulation and the modified release granulation.

In a pharmaceutical composition provided by the present disclosure, an excipient granulation provided by the present disclosure can have an average granule size that is, for example, within +/−20% of the average particle size of a pharmaceutical granulation with which the excipient granulation is combined, within +1-15%, within +/−10%, or within +1-5% of the average particle size of a pharmaceutical granulation with which the excipient granulation is combined.

In a pharmaceutical composition provided by the present disclosure, an excipient granulation provided by the present disclosure can have an average bulk density that is, for example, within +/−20% of the average bulk density of a pharmaceutical granulation with which the excipient granulation is combined, within +/−15%, within +/−10%, within +/−5%, or within less than +/−2% of the average bulk density of a pharmaceutical granulation with which the excipient granulation is combined.

In a pharmaceutical composition provided by the present disclosure, an excipient granulation provided by the present disclosure can have an average tapped density that is, for example, within +/−20% of the average tapped density of a pharmaceutical granulation with which the excipient granulation is combined, within +/−15%, within +/−10%, within +/−5%, or within less than +/−2% of the average tapped density of a pharmaceutical granulation with which the excipient granulation is combined.

In a pharmaceutical composition provided by the present disclosure, an excipient granulation provided by the present disclosure can have a Hausner ratio that is, for example, within +/−20% of the average Hausner ratio of a pharmaceutical granulation with which the excipient granulation is combined, within +/−15%, within +/−10%, within +/−5%, or within less than +/−2% of the Hausner ratio of a drug-containing granulation with which the excipient granulation is combined.

In a pharmaceutical composition provided by the present disclosure, an excipient granulation provided by the present disclosure can have a compressibility index that is, for example, within +/−20% of the compressibility index of a pharmaceutical granulation with which the excipient granulation is combined, within +/−15%, within +/−10%, within +/−5%, or within less than +/−2% of the compressibility index of a pharmaceutical granulation with which the excipient granulation is combined.

In a pharmaceutical composition provided by the present disclosure, an excipient granulation provided by the present disclosure can have a Flodex value that is, for example, within +/−20% of the Flodex value of a pharmaceutical granulation with which the excipient granulation is combined, within +/−15%, within +/−10%, or within +/−5%, or within less than +/−5% of the Flodex value of a pharmaceutical granulation with which the excipient granulation is combined.

In a pharmaceutical composition provided by the present disclosure, an excipient granulation provided by the present disclosure can have a friability that is, for example, within +/−20% of the friability of a pharmaceutical granulation with which the excipient granulation is combined, within +/−15%, within +/−10%, or within +/−5%, or within less than +/−2% of the friability of a pharmaceutical granulation with which the excipient granulation is combined.

A pharmaceutical composition provided by the present disclosure can comprise an immediate release pharmaceutical granulation and a modified release pharmaceutical granulation.

A pharmaceutical composition can comprise, for example, from 20 wt % to 30 wt % of an immediate release pharmaceutical granulation, from 22 wt % to 28 wt % or from 24 wt % to 26 wt % of an immediate release pharmaceutical granulation, where wt % is based on the total weight of the pharmaceutical composition.

A pharmaceutical composition can comprise, for example, from 62 wt % to 72 wt % of an immediate release pharmaceutical granulation, from 64 wt % to 70 wt % or from 66 wt % to 68 wt % of an immediate release pharmaceutical granulation, where wt % is based on the total weight of the pharmaceutical composition.

A pharmaceutical composition can comprise, for example, from 2 wt % to 12 wt % of an excipient granulation, from 4 wt % to 10 wt % or from 6 wt % to 8 wt % of an excipient granulation, where wt % is based on the total weight of the pharmaceutical composition.

A pharmaceutical composition can comprise, for example, from 2.5 wt % to 12.5 wt % of the excipient granules, from 3.5 wt % to 11.5 wt %, from 4.5 wt % to 10.5 wt %, from 5.5 wt % to 9.5 wt %, from 6.5 wt % to 8.5 wt %, from 7.0 wt % to 8.0 wt %, or 7.5 wt %, where wt % is based on the total weight of the pharmaceutical composition.

A pharmaceutical composition can comprise, for example, from 20 wt % to 30 wt % of the immediate release pharmaceutical granulation, from 62 wt % to 72 wt % of the modified release pharmaceutical granulation, and from 2 wt % to 12 wt % of the excipient granulation, where wt % is based on the total weight of the pharmaceutical composition.

A pharmaceutical composition can comprise, for example, from 22 wt % to 28 wt % of the immediate release pharmaceutical granulation, from 64 wt % to 72 wt % of the modified release pharmaceutical granulation, and from 4 wt % to 10 wt % of the excipient granulation, where wt % is based on the total weight of the pharmaceutical composition.

A pharmaceutical composition can comprise, for example, from 24 wt % to 26 wt % of the immediate release pharmaceutical granulation, from 66 wt % to 70 wt % of the modified release pharmaceutical granulation, and from 6 wt % to 9 wt % of the excipient granulation, where wt % is based on the total weight of the pharmaceutical composition.

The pharmaceutical composition can comprise, for example, from 24 wt % to 38 wt % of the 4-((L-valyl)oxy)butanoic acid in the immediate release component and from 62 wt % to 76 wt % of the 4-((L-valyl)oxy)butanoic acid in the modified release component, where wt % is based on the total weight of the 4-((L-valyl)oxy)butanoic acid in the pharmaceutical composition.

The pharmaceutical composition can comprise, for example, from 26 wt % to 36 wt % of the 4-((L-valyl)oxy)butanoic acid in the immediate release component and from 64 wt % to 74 wt % of the 4-((L-valyl)oxy)butanoic acid in the modified release component, where wt % is based on the total weight of the 4-((L-valyl)oxy)butanoic acid in the pharmaceutical composition.

The pharmaceutical composition can comprise, for example, from 28 wt % to 34 wt % of the 4-((L-valyl)oxy)butanoic acid in the immediate release component and from 66 wt % to 72 wt % of the 4-((L-valyl)oxy)butanoic acid in the modified release component, where wt % is based on the total weight of the 4-((L-valyl)oxy)butanoic acid in the pharmaceutical composition.

The pharmaceutical composition can comprise, for example, from 30 wt % to 32 wt % of the 4-((L-valyl)oxy)butanoic acid in the immediate release component and from 68 wt % to 70 wt % of the 4-((L-valyl)oxy)butanoic acid in the modified release component, where wt % is based on the total weight of the 4-((L-valyl)oxy)butanoic acid in the pharmaceutical composition.

In a pharmaceutical composition, the ratio of the weight of 4-((L-valyl)oxy)butanoic acid in the modified release pharmaceutical granulation to the weight of 4-((L-valyl)oxy)butanoic acid in the immediate release pharmaceutical granulation can be, for example, from 1.7 to 3.7, from 1.9 to 3.5, from 2.1 to 3.3, from 2.3 to 3.1, or from 2.5 to 2.9.

In a pharmaceutical composition, the modified release pharmaceutical granulation can comprise, for example, from 64 wt % to 74 wt % of the 4-((L-valyl)oxy)butanoic acid and the immediate release pharmaceutical granulation can comprise from 26 wt % to 36 wt % of the 4-((L-valyl)oxy)butanoic acid, wherein wt % is based on the total weight of the 4-((L-valyl)oxy)butanoic acid in the pharmaceutical composition.

A pharmaceutical composition can further comprise one or more additional excipients that can be, for example, in powder form, and which are independent of the excipient granulation. The one or more additional excipients can comprise pharmaceutically acceptable excipients.

The one or more additional excipients can facilitate dispersion of an excipient granulation and a pharmaceutical granulation and/or minimize or prevent agglomeration of an excipient granulation and a pharmaceutical granulation.

The one or more additional excipients can comprise, for example an antistatic agent.

Examples of suitable antistatic agents include talc (hydrous magnesium silicate), magnesium stearate, and silicon dioxide.

A pharmaceutical composition can comprise hydrous magnesium silicate (talc).

A pharmaceutical composition can comprise, for example, from 0.1 wt % to 1.2 wt %, from 0.2 wt % to 1.1 wt %, from 0.3 wt % to 1.0 wt %, or from 0.4 wt % to 0.9 wt % of an antistatic agent such as hydrous magnesium silicate, where wt % is based on the total weight of the pharmaceutical composition.

A pharmaceutical composition and an oral pharmaceutical composition provided by the present disclosure can comprise a therapeutically effective dose of 4-((L-valyl)oxy)butanoic acid.

For a 14.5 g dose of 4-((L-valyl)oxy)butanoic acid, 10 g of 4-((L-valyl)oxy)butanoic acid can be in the modified release pharmaceutical granulation, and 4.5 g of 4-((L-valyl)oxy)butanoic acid can be in the immediate release pharmaceutical granulation.

For a 14.5 g dose of 4-((L-valyl)oxy)butanoic acid, from 8 g to 12 g of 4-((L-valyl)oxy)butanoic acid can be in the modified release pharmaceutical granulation, and 2.5 g to 6.5 g of 4-((L-valyl)oxy)butanoic acid can be in the immediate release pharmaceutical granulation.

A 14.5 g dose of 4-((L-valyl)oxy)butanoic acid can comprise 13.34 g of the modified release pharmaceutical granulation, 5 g of the immediate release pharmaceutical granulation, 1.50 g of the excipient granulation, and 0.10 g of an antistatic agent such as talc.

A 14.5 g dose of 4-((L-valyl)oxy)butanoic acid can comprise from 11.34 g to 15.35 g of the modified release pharmaceutical granulation, 3 g to 7 g of the immediate release pharmaceutical granulation, from 1 g to 2 g of the excipient granulation, and from 0.05 g to 0.15 g of an antistatic agent such as talc.

A dose of 4-((L-valyl)oxy)butanoic acid can comprise, for example, from 1 g to 30 g of 4-((L-valyl)oxy)butanoic acid, such as from 2 g to 25 g, from 4 g to 20 g, from 5 g to 20 g, or from 10 g to 15 g of 4-((L-valyl)oxy)butanoic acid.

A pharmaceutical composition can comprise a unit dose of 4-((L-valyl)oxy)butanoic acid, where a unit dose refers to a single dose that is capable of treating an intended disease or symptom of a disease.

An oral pharmaceutical composition provided by the present disclosure can comprise a viscous aqueous solution and an active pharmaceutical ingredient. An oral pharmaceutical composition can be formed by combining a pharmaceutical composition with an aqueous solution such as water. An oral pharmaceutical composition orally ingestible by a patient.

An oral pharmaceutical composition provided by the present disclosure can comprise a oral pharmaceutical composition for oral ingestion prepared by mixing a pharmaceutical composition provided by the present disclosure with water or an aqueous solution to provide a viscous solution comprising a modified release pharmaceutical granulation provided by the present disclosure dispersed and suspended in the viscous solution. The viscous solution can comprise the constituents of the excipient granulation dissolved in water or the aqueous solution. The viscous solution can also comprise 4-((L-valyl)oxy)butanoic acid dissolved in the viscous solution where the comprise 4-((L-valyl)oxy)butanoic acid is provided by the partially or fully dissolved immediate release pharmaceutical granulation. The viscous solution can further comprise a partially dissolved immediate release pharmaceutical granulation suspended and dispersed in the viscous solution.

An oral pharmaceutical composition provided by the present disclosure can comprise a viscous aqueous solution. The oral pharmaceutical composition is intended for oral administration.

A viscous aqueous solution can be prepared by combining and mixing an excipient granulation provided by the present disclosure with an aqueous solution.

When excipient granulation is mixed with water, the contents of the excipient granulation provide a concentration, for example, from 0.005 g/mL to 0.060 g/mL of the excipient granules, from 0.010 g/mL to 0.055 g/mL, from 0.015 g/mL to 0.050 g/mL, from 0.015 g/mL to 0.045 g/mL, from 0.02 g/mL to 0.04 g/mL or from 0.025 g/mL to 0.035 g/mL of the excipient granulation, where g/mL is based on the grams of the excipient granulation mixed with the volume of water.

For example, an oral pharmaceutical composition can be prepared, for example, by mixing from 0.2 g to 2.2 g of the excipient granulation in 40 mL of water, from 0.4 g to 2.0 g, from 0.6 g to 1.8 g, from 0.8 g to 1.6 g, from 1.0 g to 1.4 g of the excipient granulation in 40 mL of water.

An oral pharmaceutical composition provided by the present disclosure can be prepared by combining an excipient granulation and a pharmaceutical granulation with water. The excipient granulation and the pharmaceutical granulation can be combined with water either simultaneously or sequentially. For example, an excipient granulation can first be combined with water to form a viscous solution, and a pharmaceutical granulation subsequently combined and mixed with the viscous solution. Alternatively, a pharmaceutical granulation can first be combined with water, and an excipient granulation subsequently added to the aqueous solution. As another example, each of the excipient granulation, the immediate release pharmaceutical granulation, and the modified release pharmaceutical granulation can be added in any order, simultaneously, or independently to an aqueous solution such as water to provide an oral pharmaceutical composition.

An oral pharmaceutical composition can comprise a viscous solution and 4-((L-valyl)oxy)butanoic acid dissolved in the viscous solution and/or a pharmaceutical granulation comprising 4-((L-valyl)oxy)butanoic acid suspended in the viscous solution.

An oral pharmaceutical composition can comprise an immediate release component and a modified release component. An immediate release component can comprise 4-((L-valyl)oxy)butanoic acid dissolved in the viscous aqueous solution, an immediate release pharmaceutical granulation suspended in the viscous solution, or a combination thereof. A modified release component can comprise a modified release pharmaceutical granulation suspended in the viscous solution.

An immediate release component and a modified release component can comprise 4-((L-valyl)oxy)butanoic acid.

An oral pharmaceutical composition can have a viscosity, for example, from 50 cP to 1,100 cP or from 50 cP to 600 cP at a temperature from 20° C. to 25° C., such as 23° C., determined using a No. 62 spindle at a speed of 12 rpm according to USP <912>, Method 1c. An oral pharmaceutical composition can have a viscosity, for example, greater than 50 cP, greater than 100 cP, greater than 200 cP, greater than 400 cP, greater than 800 cP or greater than 800 cP at a temperature from 20° C. to 25° C., such as 23° C., determined using a No. 62 spindle at a speed of 12 rpm according to USP <912>, Method 1c. An oral pharmaceutical composition can have a viscosity, for example, of less than 1,000 cP, less than 800 cP, less than 600 cP, less than 400 cP, less than 200 cP, or less than 100 cP, at a temperature from 20° C. to 25° C., such as 23° C., determined using a No. 62 spindle at a speed of 12 rpm according to USP <912>, Method 1c.

An oral pharmaceutical composition provided by the present disclosure can have a viscosity, for example, from 350 cp to 550 cp in 30 seconds after mixing with water, from 775 cP to 975 cp in 60 seconds, and from 950 cP to 1,150 cP after mixing with water, where the viscosity is determined according to USP <912> Method 1c, using a No. 62 spindle at a speed of 12 rpm, at a temperature of 23° C., and a concentration of 40 g/L.

An oral pharmaceutical composition provided by the present disclosure can have a viscosity, for example, from 400 cp to 500 cp in 30 seconds after mixing with water, from 825 cP to 925 cp in 60 seconds, and from 1,000 cP to 1,100 cP after mixing with water, where the viscosity is determined according to USP <912> Method 1c, using a No. 62 spindle at a speed of 12 rpm, at a temperature of 23° C., and a concentration of 40 g/L.

An oral pharmaceutical composition can have a viscosity, for example, from 10 cP to 1,000 cP or from 20 cP to 800 cP at a temperature from 20° C. to 25° C. such as 23° C. An oral pharmaceutical composition can have a viscosity, for example, greater than 10 cP, greater than 20 cP, greater than 50 cP, greater than 100 cP, greater than 200 cP, greater than 400 cP, greater than 600 cP, or greater than 800 cP at a temperature from 20° C. to 25° C. such as 23° C. An oral pharmaceutical composition can have a viscosity, for example, of less than 1,000 cP, less than 800 cP, less than 600 cP, less than 400 cP, or less than 200 cP, at a temperature from 20° C. to 25° C. such as 23° C. Viscosity is determined using a Brookfield Digital Viscometer Model LVDV-I+ equipped with a Brookfield SC4-18 spindle at a spindle speed of 30 rpm. The oral pharmaceutical composition can be prepared by combining and mixing, for example, from 10 g to 20 g of a pharmaceutical composition with from 30 mL to 50 mL of water. The oral pharmaceutical composition can be prepared by combining and mixing, for example, from 12 g to 18 g of a pharmaceutical composition with from 35 mL to 45 mL of water. The oral composition can have a concentration of the pharmaceutical composition, for example, from 0.20 g/mL to 0.70 g/mL, such as from 0.30 g/mL to 0.42 g/mL, or from 0.32 g/mL to 0.40 g/mL.

Following reconstitution in water, an oral pharmaceutical composition can be pourable and have a viscosity similar to that of drinkable yogurt.

A pharmaceutical composition can comprise an excipient granulation and a pharmaceutical granulation comprising an immediate-release pharmaceutical granulation and a modified-release pharmaceutical granulation. When combined with a suitable amount of water, the excipient granulation can disintegrate to cause the viscosity of the solution to increase, the immediate release pharmaceutical granulation can dissolve to provide the active pharmaceutical ingredient dissolved in the viscous solution, and the modified-release pharmaceutical granulation can be suspended and dispersed in the viscous solution.

The modified-release pharmaceutical granulation can be configured to retain the physical integrity of the modified release granules and dissolve and/or release the 4-((L-valyl)oxy)butanoic acid over time in the gastrointestinal tract with a release profile determined by the constituents and the coating of the modified-release pharmaceutical granules or combination of modified-release pharmaceutical granulations.

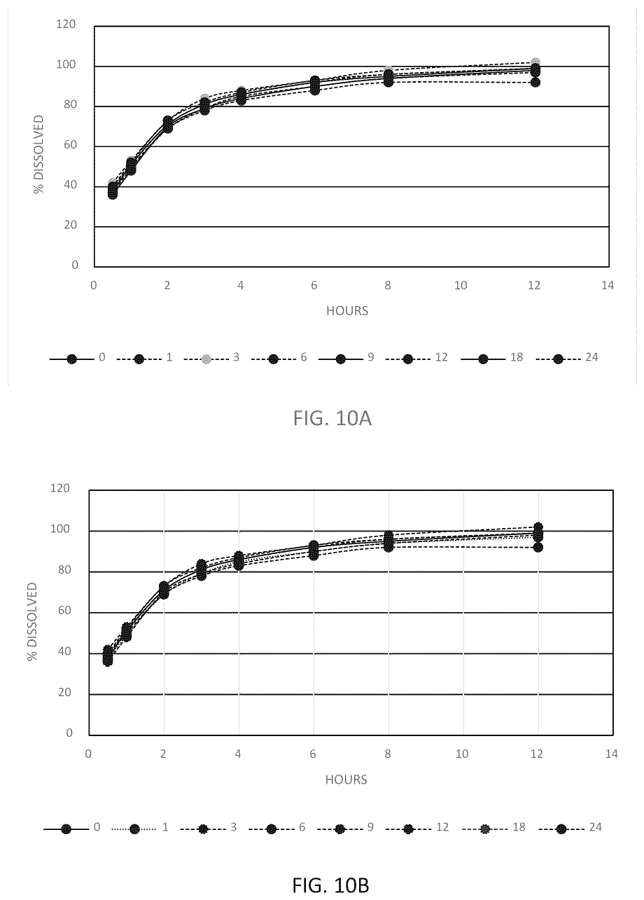

A pharmaceutical composition comprising about 7.6 wt % of an excipient granulation, about 25.2 wt % of an immediate release pharmaceutical granulation, and about 67.2 wt % of a modified release pharmaceutical granulation can exhibit a dissolution profile as shown in FIG. 10A, where the dissolution profile is determined as described in Example 6.

A pharmaceutical composition comprising about 7.6 wt % of an excipient granulation, about 25.2 wt % of an immediate release pharmaceutical granulation, and about 67.2 wt % of a modified release pharmaceutical granulation can exhibit a 4-((L-valyl)oxy)butanoic acid dissolution profile wherein from 26% to 46% is released within 0.5 hours, from 41% to 61% is released within 1 hour, from 63% to 83% is released within 2 hours, and from 73% to 93% is released within 4 hours, where the dissolution profile is determined as described in Example 6.

A pharmaceutical composition comprising about 7.6 wt % of an excipient granulation, about 25.2 wt % of an immediate release pharmaceutical granulation, and about 67.2 wt % of a modified release pharmaceutical granulation can exhibit a 4-((L-valyl)oxy)butanoic acid dissolution profile wherein from 310% to 41% is released within 0.5 hours, from 46% to 56% is released within 1 hour, from 68% to 78% is released within 2 hours, and from 78% to 88% is released within 4 hours, where the dissolution profile is determined as described in Example 6.

Following storage of a pharmaceutical composition provided by the present disclosure at 5±3° C. or at 25±2° C./60±5% RH sealed in high barrier pouch for 18 months, the 4-((L-valyl)oxy)butanoic acid dissolution profile is similar to the 4-((L-valyl)oxy)butanoic acid dissolution profile prior to storage.

Following storage of a pharmaceutical composition provided by the present disclosure at 5±3° C. or at 25±2° C./60±5% RH sealed in high barrier pouch for 18 months, the reconstitution time is about 15 seconds.

Following storage of a pharmaceutical composition provided by the present disclosure at 5±3° C. or at 25±2° C./60±5% RH sealed in high barrier pouch for 18 months, the 4-((L-valyl)oxy)butanoic acid content is greater than 96% of the 4-((L-valyl)oxy)butanoic acid prior to storage.

A pharmaceutical composition provided by the present disclosure can have an initial total impurity content of 0.2% or less, and an L-valine impurity content of less than 0.2%.

Following storage of a pharmaceutical composition provided by the present disclosure at 5±3° C. sealed in high barrier pouch for 18 months, the total impurity content is 0.3% or less and following storage at or at 25±2° C./60±5% RH sealed in high barrier pouch for 18 months, the total impurity content is 0.6% or less.

Following storage of a pharmaceutical composition provided by the present disclosure at 5±3° C. sealed in high barrier pouch for 18 months, the L-valine impurity content is 0.3% or less and following storage at or at 25±2° C./60±5% RH sealed in high barrier pouch for 18 months, the L-valine impurity content is 0.4% or less.

A method provided by the present disclosure can comprise combining an excipient granulation, an immediate release pharmaceutical granulation, and a modified-release pharmaceutical granulation with water to provide an oral pharmaceutical composition in the form of a viscous solution including a modified release pharmaceutical granulation.

A method provided by the present disclosure can comprise a user opening a package comprising an excipient granulation provided by the present disclosure, pouring the excipient granulation into an aqueous solution, and mixing the excipient granules in the aqueous solution to provide a viscous solution.

A method provided by the present disclosure can comprise a user opening a package comprising an excipient granulation provided by the present disclosure and a pharmaceutical granulation, pouring the excipient granulation and the pharmaceutical granulation into an aqueous solution, and mixing the excipient granulation and the pharmaceutical granulation in the aqueous solution to provide a viscous aqueous solution containing 4-((L-valyl)oxy)butanoic acid dissolved in the viscous solution and/or in an immediate release granulation, and a modified release pharmaceutical granulation suspended in the viscous aqueous solution.

When combined with a suitable amount of water a pharmaceutical composition provided by the present disclosure is configured to provide a solution having a viscosity capable of suspending a pharmaceutical granulation. The suspension can comprise pharmaceutical granules suspended in the viscous solution.

The aqueous solution can have a volume, for example, from 25 mL to 250 mL, from 25 mL to 200 mL, from 25 mL to 150 mL, from 25 mL to 100 mL, or from 25 mL to 75 mL.

The aqueous solution can comprise water such as tap water.

A pharmaceutical composition provided by the present disclosure can be included in a kit that can be used to administer 4-((L-valyl)oxy)butanoic acid to a patient for therapeutic purposes. A kit can include a pharmaceutical composition, instructions for preparing an oral pharmaceutical composition suitable for oral administration to a patient, and instructions for administering the oral pharmaceutical composition to the patient. The kit can comprise a pharmaceutical composition provided by the present disclosure and instructions for combining the contents of the kit with an aqueous solution to provide an oral pharmaceutical composition and with instructions or administering the oral pharmaceutical composition to a patient.

A kit can comprise a primary package and a secondary package containing a pharmaceutical composition.

Pharmaceutical compositions or granulations can be included in primary package such as a container, sachet, blister pack, package, or dispenser together with instructions for preparation of the oral pharmaceutical composition and administration.

Instructions supplied with a kit may be printed and/or supplied, for example, as an electronic-readable medium, a video cassette, an audiotape, a flash memory device, or may be published on an internet web site or distributed to a patient and/or health care provider as an electronic communication.

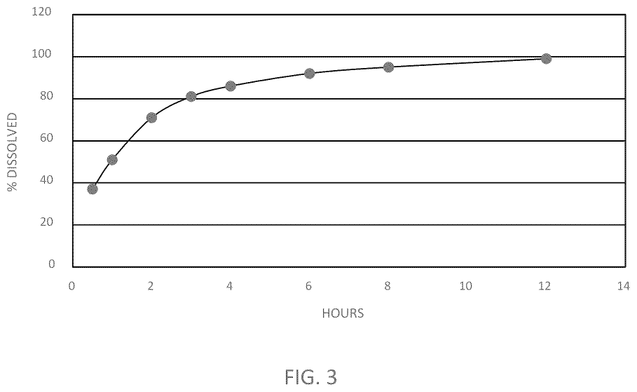

A pharmaceutical granulation comprising an immediate release granulation and a modified release granulation and a pharmaceutical composition comprising an excipient granulation, an immediate release granulation, and a modified release granulation can exhibit a dissolution profile of 4-((L-valyl)oxy)butanoic acid as substantially shown in FIG. 3 The constituents of the pharmaceutical composition and the dissolution conditions are provided in Example 2.

A pharmaceutical granulation comprising an immediate release granulation and a modified release granulation and a pharmaceutical composition comprising an excipient granulation, an immediate release granulation, and a modified release granulation can exhibit a dissolution profile in which from 40% to 60% of the 4-((L-valyl)oxy)butanoic acid is released into the dissolution medium within 1 hour, from 60% to 80% is released within 2 hours, from 80% to 100% is released within 5 hours, and/or from 90% to 100% is released into the dissolution medium within 8 hours, where % is based on the total weight of 4-((L-valyl)oxy)butanoic acid in the pharmaceutical composition (immediate release and modified release pharmaceutical granulations). The constituents of the pharmaceutical composition and the dissolution conditions are provided in Examples 2 and 4.

A pharmaceutical granulation comprising an immediate release granulation and a modified release granulation, and a pharmaceutical composition comprising an excipient granulation, an immediate release granulation, and a modified release granulation can exhibit a dissolution profile in which from 45% to 55% of the 4-((L-valyl)oxy)butanoic acid is released into the dissolution medium within 1 hour, from 65% to 75% is released within 2 hours, from 85% to 95% is released within 5 hours, and/or from 90% to 100% is released into the dissolution medium within 9 hours, where % is based on the total amount of 4-((L-valyl)oxy)butanoic acid in the pharmaceutical granulation (immediate release and modified release pharmaceutical granulations). The 4-((L-valyl)oxy)butanoic acid dissolution conditions are provided in Examples 2 and 4.

A pharmaceutical composition comprising an excipient granulation, an immediate release granulation, and a modified release granulation can exhibit a 4-((L-valyl)oxy)butanoic acid dissolution profile that is bioequivalent to the dissolution profile shown in FIG. 5. The 4-((L-valyl)oxy)butanoic acid dissolution conditions are provided in Example 4.

A product provided by the present disclosure can comprise an excipient granulation provided by the present disclosure contained within a package. The package can be any suitable package such as a container, a sachet, a blister pack, or an ampoule.

A pharmaceutical product provided by the present disclosure can comprise an excipient granulation and a pharmaceutical granulation contained within a primary package.

A primary package can include any package suitable for use with pharmaceutical granulations. Examples of suitable primary packages include blister packs, sachets, pouches, capsules, ampoules, and vials.

In a pharmaceutical product, the excipient granulation and the pharmaceutical granulation can be contained within the same primary package.

In a pharmaceutical product, the excipient granulation and the pharmaceutical granulation can be contained within separate primary packages.

In a pharmaceutical product ta immediate release pharmaceutical granulation and a modified release pharmaceutical granulation can be contained within separate primary packages or within the same primary package.

In a pharmaceutical product, a package can contain a unit dose of 4-((L-valyl)oxy)butanoic acid or less than a unit dose of 4-((L-valyl)oxy)butanoic acid. In a pharmaceutical product comprising packages such as sachets comprising less than a unit dose of the active pharmaceutical ingredient, the pharmaceutical product can contain two or more packages containing the 4-((L-valyl)oxy)butanoic acid such that the total amount of the 4-((L-valyl)oxy)butanoic acid t in the two or more primary packages makes up a unit dose of 4-((L-valyl)oxy)butanoic acid.

A pharmaceutical product can comprise one or compartments containing an excipient granulation and the pharmaceutical granulation. A compartment can be a blister pack.

The excipient granulation and the pharmaceutical granulation can be contained within the same compartment of a package.

Each of the excipient granulation and the pharmaceutical granulation can be contained within separate, independent compartments of a common package. For example, a package can comprise a first compartment containing the excipient granulation and a second compartment containing the pharmaceutical granulation.

A pharmaceutical product can comprise a compartment containing the excipient granulation and a second compartment containing the pharmaceutical granulation where the compartments are configured to be mixed before or during combination with an aqueous solution.

A pharmaceutical product provided by the present disclosure can comprise a first package comprising an excipient granulation provided by the present disclosure and a second package comprising a pharmaceutical granulation.

A pharmaceutical product can comprise a sachet that contains both an excipient granulation provided by the present disclosure and a pharmaceutical granulation.

A pharmaceutical product can comprise, for example, from 0.5 g to 30 g of a combination of an excipient granulation and a pharmaceutical granulation, such as from 1 g to 25 g, from 1 g to 20 g, from 2 g to 15 g, from 2 g to 10 g, from 2 g to 5 g, or from 1 g to 5 g of a combination of an excipient granulation and a pharmaceutical granulation.

A pharmaceutical product provided by the present disclosure can comprise a unit dose of 4-((L-valyl)oxy)butanoic acid.

A unit dose of 4-((L-valyl)oxy)butanoic acid can be provided in one or more packages such as one or more sachets.

A pharmaceutical product provided by the present disclosure can comprise more than one unit dose of 4-((L-valyl)oxy)butanoic acid. Each of the one or more unit doses can be provided in separate packages such as in separate sachets.

The unit dose can be suitable for treating a disease of a patient.

An oral pharmaceutical composition provided by the present disclosure can provide a therapeutically effective amount of γ-hydroxybutyric acid over a period of time in the plasma of a patient. For example, an oral formulation provided by the present disclosure can provide a therapeutically effective amount of γ-hydroxybutyric acid over a period of 3 hours, 6 hours, 8 hours, or 10 hours.

An oral pharmaceutical composition provided by the present disclosure can provide a therapeutically effective amount of γ-hydroxybutyric acid over a period from 4 hours to 12 hours, from 4 hours to 10 hours, or from 4 hours to 8 hours.

An oral pharmaceutical composition provided by the present disclosure can provide a therapeutically effective amount of γ-hydroxybutyric acid over a duration, for example, from 1 hour to 12 hours following oral administration, from 2 hours to 10 hours or from 4 hours to 8 hours following oral administration.

An oral pharmaceutical composition provided by the present disclosure can be a once nightly formulation. For a once nightly formulation, a patient can administer a dose of crystalline 4-((L-valyl)oxy)butanoic acid and/or γ-hydroxybutyric acid as a prodrug provided by the present disclosure before going to bed and sleep through the night such as for 6 hours or for 8 hours without having to administer a second dose during the night.

An oral pharmaceutical composition provided by the present disclosure can provide a therapeutically effective amount of γ-hydroxybutyric acid in the plasma of a patient. An oral pharmaceutical composition provided by the present disclosure can provide a therapeutically effective amount of γ-hydroxybutyric acid in the plasma of a patient for a period of 4 hours, 6 hours, 8 hours, or 10 hours following oral administration of the modified release oral pharmaceutical composition.

An oral pharmaceutical composition provided by the present disclosure can provide a plasma concentration of γ-hydroxybutyric acid, for example, greater than 10 μg/mL for more than 4 hours, for more than 6 hours, for more than 8 hours, or for more than 10 hours following oral administration of the oral pharmaceutical composition.

An oral pharmaceutical composition provided by the present disclosure can provide a plasma concentration of γ-hydroxybutyric acid, for example, greater than 15 μg/mL for more than 4 hours, for more than 6 hours, for more than 8 hours, or for more than 10 hours following oral administration of the oral pharmaceutical composition.

An oral formulation provided by the present disclosure can provide a therapeutically effective amount of Cₘₐₓ to Cₘᵢₙ ratio of γ-hydroxybutyric acid in the plasma of a patient, for example, from less than 3 or less than 2 for a duration of 4 hours, 6 hours, 8 hours, or 10 hours following oral administration of the modified release oral pharmaceutical composition.

An oral pharmaceutical composition provided by the present disclosure can comprise 4-((L-valyl)oxy)butanoic acid such as crystalline 4-((L-valyl)oxy)butanoic acid and can comprise, for example, 0.5 g-equivalents of γ-hydroxybutyric acid, 1 g-equivalents, 2 g-equivalents, 3 g-equivalents, 4 g-equivalents, 5 g-equivalents, 6 g-equivalents, 7 g-equivalents, 8 g-equivalents, 9 g-equivalents, 10 g-equivalents, 11 g-equivalents, or 12 g-equivalents of γ-hydroxybutyric acid. An oral pharmaceutical composition provided by the present disclosure can comprise a crystalline 4-((L-valyl)oxy)butanoic acid and can comprise, for example, from 0.5 g-equivalents to 12 g-equivalents of γ-hydroxybutyric acid, from 1 g-equivalents to 12 g-equivalents, from 2 g-equivalents to 12 g-equivalents, from 3 g-equivalents to 11 g-equivalents, from 4 g-equivalents to 10 g-equivalents, or from 5 g-equivalents to 9 g-equivalents of γ-hydroxybutyric acid. An oral pharmaceutical composition provided by the present disclosure can comprise, for example, greater than 0.5 g-equivalents of γ-hydroxybutyric acid, greater than 1 g-equivalents, greater than 3 g-equivalents, greater than 5 g-equivalents, greater than 7 g-equivalents, greater than 9 g-equivalents, or greater than 11 g-equivalents of γ-hydroxybutyric acid.

An oral pharmaceutical composition provided by the present disclosure can comprise a suspension such as an aqueous suspension of an immediate release pharmaceutical granulation and a modified release granulation comprising 4-((L-valyl)oxy)butanoic acid. The immediate release pharmaceutical granulation and the core of the modified release pharmaceutical granulation can comprise greater than 90 wt % such as greater than 94 wt %, or greater than 96 wt % of 4-((L-valyl)oxy)butanoic acid, where wt % is based on the total weight of the immediate release pharmaceutical granulation or the core of the modified release pharmaceutical granulation.

An oral pharmaceutical composition provided by the present disclosure can be provided, for example, as a sachet or package containing an immediate release component such as an immediate release pharmaceutical granulation comprising 4-((L-valyl)oxy)butanoic acid and a modified release component such as modified release pharmaceutical granulation comprising 4-((L-valyl)oxy)butanoic acid. A sachet or package can be provided in different dose equivalents of γ-hydroxybutyric acid such as 0.5 g, 1 g, 2 g, 3 g, 4 g, 5 g, 6 g, 7 g, 8 g, 9 g, 10 g, 11 g, 10 g, 12 g, 15 g, or 20 g-equivalents of γ-hydroxybutyric acid.

An oral dosage form such as a coated granulation comprising immediate release granules and modified release granules can be combined, for example, with water to provide an orally ingestible dosage form.

Following oral administration of an oral formulation provided by the present disclosure and absorption of 4-((L-valyl)oxy)butanoic acid in the gastrointestinal tract can be metabolized in the systemic circulation of a patient to provide γ-hydroxybutyric acid.

Methods provided by the present disclosure include providing a therapeutically effective amount of γ-hydroxybutyric acid in the systemic circulation of a patient for treating a disease or disorder in a patient or a symptom of a disease or disorder in a patient comprising administering to a patient an oral formulation provided by the present disclosure comprising 4-((L-valyl)oxy)butanoic acid.

A suitable dose of 4-((L-valyl)oxy)butanoic acid can provide, for example, a dose of from 1 gram-equivalents to 12 gram-equivalents of γ-hydroxybutyric acid, such as a dose from 1 gram-equivalents to 10 gram-equivalents, from 2 gram-equivalents to 9 gram-equivalents, from 3 gram-equivalents to 8 gram-equivalents, or from 4 gram-equivalents to 7 gram-equivalents of γ-hydroxybutyric acid.

It will be understood, however, that the specific dose level and frequency of dosage for any particular patient may be varied and will depend upon a variety of factors including: the metabolic stability and length of action, the age, body weight, general health, gender, diet, mode and time of administration, rate of excretion, drug combination, the severity of the particular condition, and the host undergoing therapy.

When initiating treatment with 4-((L-valyl)oxy)butanoic acid, titration up to an adequate plasma concentration can facilitate obtaining positive results and avoid adverse effects. For example, a dose can be 4.5 g-equivalents of γ-hydroxybutyric acid divided into 2 equal doses of 2.25 g, the first taken at bedtime and the second taken 2.5 hours to 4 hours later. The starting dose can be decreased to 3.0 g/day or increased to as high as 9.0 g/day in increments of 1.5 g/day (0.75 g per dose). A dose can provide an effective eight hours of sleep but, at the end of eight hours, little of the γ-hydroxybutyric acid will remain in the plasma of a patient.

An oral pharmaceutical formulation provided by the present disclosure can be configured to provide for once a night dosing, once a day dosing (QD), twice a day dosing (BID), three times a day dosing (TID), or four times a day dosing (QID). For example, a pharmaceutical composition comprising a modified release granulation can release substantially 100% of crystalline 4-((L-valyl)oxy)butanoic acid over a 24-hour duration, a 12-hour duration, an 8-hour duration, or a 4-hour duration.

4-((L-Valyl)oxy)butanoic acid is a prodrug of γ-hydroxybutyric acid. 4-((L-Valyl)oxy)butanoic acid can be used to treat any disease or disorder that is known to be treated by γ-hydroxybutyric acid or is determined to be treated by γ-hydroxybutyric acid.

For example, 4-((L-valyl)oxy)butanoic acid can be used to treat narcolepsy, excessive daytime sleepiness, cataplexy, excessive daytime sleepiness associated with narcolepsy, excessive daytime sleepiness associated with Parkinson's disease, excessive daytime sleepiness associated with multiple sclerosis, cataplexy associated with narcolepsy, fatigue, fatigue associated with Parkinson's disease, fatigue associated with multiple sclerosis, and fibromyalgia.

4-((L-Valyl)oxy)butanoic acid can be used to treat REM sleep behavior disorder, spasmodic dystonia, schizophrenia, insomnia, insomnia associated with schizophrenia, idiopathic hypersomnia, chronic fatigue syndrome, cluster headache, symptoms of Alzheimer's disease, essential tremor, post-traumatic stress syndrome, insomnia associated with post-traumatic stress syndrome, and anxiety.

4-((L-Valyl)oxy)butanoic acid can be used to treat excessive daytime sleepiness associated with narcolepsy, excessive daytime sleepiness associated with Parkinson's disease, excessive daytime sleepiness associated with multiple sclerosis, cataplexy associated with narcolepsy, fatigue in a patient with Parkinson's disease, fatigue in a patient with multiple sclerosis, or fibromyalgia.

4-((L-Valyl)oxy)butanoic acid and pharmaceutical compositions thereof can be used to treat a sleep disorders such as apnea, a sleep time disturbance, narcolepsy, cataplexy, sleep paralysis, hypnagogic hallucination, sleep arousal, insomnia, and nocturnal myoclonus.

4-((L-Valyl)oxy)butanoic acid or a pharmaceutical composition thereof can be used to treat a disease selected from narcolepsy, cataplexy, cataplexy with narcolepsy, excessive daytime sleepiness, a sleep disorder associated with Parkinson's disease, symptoms of Parkinson's disease, a neurodegenerative disease, sleep disturbance syndrome, fatigue, improving nocturnal sleep, hypnagogic hallucinations, sleep paralysis, fragmented sleep, alcohol withdrawal and dependence, obstructive sleep apnea syndrome, insomnia, insomnia associated with schizophrenia, sleep ignition and maintenance disorders, chronic fatigue syndrome, essential tremor, hemiplegia in patients with alternating hemiplegia of childhood, sedative abuse, and binge eating disorder.

For certain methods of treatment the effectiveness of the treatment can be measured by one or more of the following criteria: increase in the mean sleep latency such as determined the Maintenance of Wakefulness Test (MWT); improvement in the Clinical Global Impression (CGI) rating of sleepiness; decrease in the number of cataplexy attacks (NCA) such as determined from the cataplexy frequency item in the Sleep and Symptoms Daily Diary; decrease in disturbed nocturnal sleep (DNS), the disturbed nocturnal events or the adverse respiratory events such as determined by polysomnographic (PSG) measures of sleep fragmentation; decrease in excessive daytime sleepiness (EDS) such as measured by patient report via the Epworth Sleepiness Scale (ESS); decrease in daytime sleepiness as measured by the Maintenance of Wakefulness Test based on EEG measures of wakefulness; decrease PSG transitions from N/2 to N/3 and REM sleep to wake and N1 sleep such a determined as described in the AASM Manual for the Scoring of Sleep and Associated Events; decrease in the number of arousals or wakenings such as determined from a PSG as defined by the American Academy of Sleep Medicine; improvement in sleep quality such as determined using (i) the Sleep and Symptom Daily Diary, (ii) Visual Analog Scale (VAS) for sleep quality and sleep diary, and/or (iii) VAS for the refreshing nature of sleep; and decrease in the Hypnagogic Hallucinations (HH) or sleep paralysis (SP) symptoms in NT1 narcolepsy patients such as measured by the Sleep and Symptom Daily Diary.

Type 1 Narcolepsy (NT1) refers to narcolepsy characterized by excessive daytime sleepiness (“EDS”) and cataplexy. Type 2 Narcolepsy (NT2) refers to narcolepsy characterized by excessive daytime sleepiness without cataplexy. A diagnosis of narcolepsy (with or without cataplexy) can be confirmed by one or a combination of (i) an overnight polysomnogram (PSG) and a Multiple Sleep Latency Test (MSLT) performed within the last 2 years, (ii) a full documentary evidence confirming diagnosis from the PSG and MSLT from a sleep laboratory must be made available, (iii) current symptoms of narcolepsy including: current complaint of EDS for the last 3 months (Epworth Sleepiness Scale (ESS) greater than 10), (iv) mean Maintenance of Wakefulness Test (MWT) less than 8 minutes, (v) mean number of cataplexy events of 8 per week on baseline Sleep/Cataplexy Diary, and/or (vi) presence of cataplexy for the last 3 months and 28 events per week during screening period.

### ASPECTS

The invention is further defined by one or more of the following aspects.

Aspect 1. A pharmaceutical composition comprising a pharmaceutical granulation comprising 4-((L-valyl)oxy)butanoic acid; and an excipient granulation.

Aspect 2. The pharmaceutical composition of aspect 1, wherein the pharmaceutical granulation comprises from 1 g to 20 g of the 4-((L-valyl)oxy)butanoic acid.

Aspect 3. The pharmaceutical composition of any one of aspects 1 to 2, wherein, the pharmaceutical composition comprises from 2 wt % to 12 wt % of the excipient granulation; and wt % is based on the total weight of the pharmaceutical composition.

Aspect 4. The pharmaceutical composition of any one of aspects 1 to 3, wherein, the pharmaceutical composition comprises from 86 wt % to 98 wt % of the pharmaceutical granulation; and wt % is based on the total weight of the pharmaceutical composition.

Aspect 5. The pharmaceutical composition of any one of aspects 1 to 4, wherein the pharmaceutical granulation comprises: an immediate release pharmaceutical granulation comprising a plurality of immediate release pharmaceutical granules comprising 4-((L-valyl)oxy)butanoic acid; and a modified release pharmaceutical granulation comprising a plurality of modified release pharmaceutical granules comprising 4-((L-valyl)oxy)butanoic acid.

Aspect 6. The pharmaceutical composition of any one of aspects 1 to 5, wherein the pharmaceutical granulation comprises: from 17 wt % to 37 wt % of the immediate release pharmaceutical granulation; and from 64 wt % to 74 wt % of the modified release pharmaceutical granulation, wherein wt % is based on the total weight of the pharmaceutical granulation.

Aspect 7. The pharmaceutical composition of any one of aspects 1 to 6, wherein, the immediate release pharmaceutical granulation comprises from 2.5 g to 6.5 g of 4-((L-valyl)oxy)butanoic acid; and the modified release pharmaceutical granulation comprises from 8 g to 12 g of 4-((L-valyl)oxy)butanoic acid.

Aspect 8. The pharmaceutical composition of any one of aspects 1 to 7, wherein, the immediate release pharmaceutical granulation comprises from 26 wt % to 36 wt % of the 4-((L-valyl)oxy)butanoic acid; the modified release pharmaceutical granulation comprises from 64 wt % to 74 wt % of the 4-((L-valyl)oxy)butanoic acid; and wt % is based on the total weight of 4-((L-valyl)oxy)butanoic acid in the pharmaceutical composition.

Aspect 9. The pharmaceutical composition of any one of aspects 1 to 8, wherein a ratio of the weight of 4-((L-valyl)oxy)butanoic acid in the modified release granulation to the weight of 4-((L-valyl)oxy)butanoic acid in the immediate release granulation is from 1.7 to 3.7.

Aspect 10. The pharmaceutical composition of any one of aspects 1 to 9, wherein the pharmaceutical composition comprises: from 15 wt % to 35 wt % wt % of the immediate release pharmaceutical granulation; from 55 wt % to 75 wt % of the modified release pharmaceutical granulation; and from 2 wt % to 12 wt % of the excipient granulation, wherein wt % is based on the total weight of the pharmaceutical composition.

Aspect 11. The pharmaceutical composition of any one of aspects 1 to 10, wherein the pharmaceutical composition comprises from 65 wt % to 85 wt % of the 4-((L-valyl)oxy)butanoic acid, wherein wt % is based on the total weight of the pharmaceutical composition.

Aspect 12. The pharmaceutical composition of any one of aspects 1 to 11, wherein the pharmaceutical composition has a weight from 2 g to 30 g.

Aspect 13. The pharmaceutical composition of any one of aspects 5 to 12, wherein the uncoated immediate release pharmaceutical granules comprise from 85 wt % to 95 wt % of 4-((L-valyl)oxy)butanoic acid.

Aspect 14. The pharmaceutical composition of aspect 13, wherein the uncoated immediate release pharmaceutical granules comprises from 1 wt % to 9 wt % of a water-soluble polymer.

Aspect 15. The pharmaceutical composition of aspect 14, wherein the water-soluble polymer comprises hydroxypropylmethyl cellulose.

Aspect 16. The pharmaceutical composition of any one of aspects 13 to 15, wherein the uncoated immediate release pharmaceutical granule comprises from 1 wt % to 9 wt % of an antistatic agent.

Aspect 17. The pharmaceutical composition of aspect 16, wherein the antistatic agent comprises hydrous magnesium silicate.

Aspect 18. The pharmaceutical composition of any one of aspects 13 to 17, wherein the uncoated immediate release pharmaceutical granules have a homogeneous composition.

Aspect 19. The pharmaceutical composition of any one of aspects 13 to 18, wherein the immediate release pharmaceutical granules comprise a seal coat surrounding the uncoated immediate release pharmaceutical granule.

Aspect 20. The pharmaceutical composition of any one of aspects 13 to 19, wherein the immediate release pharmaceutical granules have an average diameter from 100 μm to 600 μm, wherein the average diameter is determined by sieve analysis or laser diffraction.

Aspect 21. The pharmaceutical composition of any one of aspects 13 to 20, wherein the immediate release pharmaceutical granules have an average bulk density from 0.40 g/mL to 1.60 g/mL, wherein the average bulk density is determined using a bulk density cylinder.

Aspect 22. The pharmaceutical composition of any one of aspects 13 to 21, wherein the immediate release pharmaceutical granules have an average bulk density from 0.40 g/mL to 1.00 g/mL, wherein the average bulk density is determined using a bulk density cylinder.

Aspect 23. The pharmaceutical composition of any one of aspects 13 to 22, wherein the immediate release pharmaceutical granules comprise a seal coating.

Aspect 24. The pharmaceutical composition of any one of aspects 13 to 23, wherein the immediate release pharmaceutical granules have a sphericity from 0.90 to 1.00, wherein the sphericity is determined using wet dispersion particle shape methods or by dynamic image analysis.

Aspect 25. The pharmaceutical composition of any one of aspects 13 to 24, wherein the immediate release pharmaceutical granules have a friability from 0.9 to 1.0, wherein the average friability is determined using a sonic sifter.

Aspect 26. The pharmaceutical composition of any one of aspects 13 to 25, wherein the immediate release pharmaceutical granules are configured to completely dissolve in water within less than 5 minutes.

Aspect 27. The pharmaceutical composition of any one of aspects 13 to 26, wherein the immediate release pharmaceutical granules completely dissolve in water within less than 1 minute.

Aspect 28. The pharmaceutical composition of any one of aspects 13 to 27, wherein the immediate release pharmaceutical granules have a dissolution profile as shown in FIG. 1.

Aspect 29. The pharmaceutical composition of any one of aspects 5 to 28, wherein the modified release pharmaceutical granules comprise: an immediate release granule; and a modified release coating surrounding the immediate release granule.

Aspect 30. The pharmaceutical composition of any one of aspects 5 to 29, wherein, the modified release pharmaceutical granules comprise from 70 wt % to 90 wt % of the immediate release granule; and wt % is based on the total weight of the modified release pharmaceutical granules.

Aspect 31. The pharmaceutical composition of any one of aspects 5 to 30, wherein, the modified release pharmaceutical granules comprise from 10 wt % to 30 wt % of the modified release coating; and wt % is based on the total weight of the modified release pharmaceutical granules.

Aspect 32. The pharmaceutical composition of any one of aspects 5 to 31, wherein, the modified release pharmaceutical granules comprise from 65 wt % to 85 wt % of 4-((L-valyl)oxy)butanoic acid; and wt % is based on the total weight of the modified release pharmaceutical granules.

Aspect 33. The pharmaceutical composition of any one of aspects 29 to 32, wherein the immediate release granule comprises an uncoated immediate release pharmaceutical granule.

Aspect 34. The pharmaceutical composition of any one of aspects 29 to 33, wherein, the modified release coating comprises from 63 wt % to 83% of a water-insoluble polymer; and wt % is based on the total weight of the modified release coating.

Aspect 35. The pharmaceutical composition of aspect 34, wherein the water-insoluble polymer comprises ethylcellulose.

Aspect 36. The pharmaceutical composition of any one of aspects 29 to 35, wherein, the modified release coating comprises from 2 wt % to 6 wt % of a water-soluble polymer; and wt % is based on the total weight of the modified release coating.

Aspect 37. The pharmaceutical composition of aspect 36, wherein the water-soluble polymer comprises hydroxypropylcellulose.

Aspect 38. The pharmaceutical composition of any one of aspects 5 to 37, wherein, the modified release coating comprises from 6 wt % to 10 wt % of a lubricant; and wt % is based on the total weight of the modified release coating.

Aspect 39. The pharmaceutical composition of aspect 38, wherein the lubricant comprises dibutyl sebacate.

Aspect 40. The pharmaceutical composition of any one of aspects 5 to 39, wherein, the modified release coating comprises from 10 wt % to 20 wt % of an antistatic agent; and wt % is based on the total weight of the modified release coating.

Aspect 41. The pharmaceutical composition of aspect 40, wherein the antistatic agent comprises hydrous magnesium silicate.

Aspect 42. The pharmaceutical composition of any one of aspects 29 to 33, wherein the modified release coating comprises: a water-insoluble polymer; a water-soluble polymer; a lubricant; and an antistatic agent.

Aspect 43. The pharmaceutical composition of aspect 42, wherein the modified release coating comprises: from 63 wt % to 83 wt % of a water-insoluble polymer; from 2 wt % to wt % of a water-soluble polymer; from 6 wt % to 10 wt % of a lubricant; and from 10 wt % to 20 wt % wt % of an antistatic agent; wherein wt % is based on the total weight of the modified release coating.

Aspect 44. The pharmaceutical composition of any one of aspects 42 to 33, wherein: the water-insoluble polymer comprises ethyl cellulose; the water-soluble polymer comprises hydroxypropylcellulose; the lubricant comprises dibutyl sebacate; and the antistatic agent comprises hydrous magnesium silicate.

Aspect 45. The pharmaceutical composition of any one of aspects 29 to 44, wherein the modified release coating has a thickness from 5 μm to 40 μm.

Aspect 46. The pharmaceutical composition of any one of aspects 5 to 45, wherein the modified release pharmaceutical granules are characterized by an average particle size from 100 μm to 600 μm, wherein the average particle size is determined by sieve analysis or by laser diffraction.

Aspect 47. The pharmaceutical composition of any one of aspects 5 to 46, wherein the modified release pharmaceutical granules are configured to release the 4-((L-valyl)oxy)butanoic acid continuously with time in the gastrointestinal tract.

Aspect 48. The pharmaceutical composition of any one of aspects 5 to 47, wherein the modified release pharmaceutical granules have a dissolution profile characterized by from 40% to 60% dissolution within 1 hour, and from 60% to 80% dissolution within 2 hours, wherein dissolution is determined using a USP Type 2 dissolution apparatus with a buffered solution at pH 4.5 at a temperature of 37° C. and a paddle speed of 75 rpm.

Aspect 49. The pharmaceutical composition of any one of aspects 5 to 48, wherein the modified release pharmaceutical granules have a dissolution profile as shown in FIG. 3.

Aspect 50. The pharmaceutical composition of any one of aspects 1 to 49, wherein, the excipient granulation comprising a plurality of excipient granules; and the excipient granules comprise: a viscosifying agent; a disintegrant; and one or more additional excipients.

Aspect 51. The pharmaceutical composition of aspect 50, wherein the excipient granules comprise: from 1 wt % to 35 wt % of the viscosifying agent; from 1 wt % to 25 wt % of the disintegrant; and from 40 wt % to 98 wt % of the one or more additional excipients; wherein wt % is based on the total weight of the excipient granules.

Aspect 52. The pharmaceutical composition of aspect 50, wherein the excipient granules comprise: from 5 wt % to 15 wt % of the viscosifying agent; from 5 wt % to 20 wt % of the disintegrant; and from 65 wt % to 90 wt % of the one or more additional excipients; wherein wt % is based on the total weight of the excipient granules.

Aspect 53. The pharmaceutical composition of any one of aspects 1 to 52, wherein the excipient granules have a homogeneous composition.

Aspect 54. The pharmaceutical composition of any one of aspects 1 to 53, wherein the excipient granules have an average particle size from 100 μm to 600 μm, where the particle size is determined by sieve analysis or laser diffraction.

Aspect 55. The pharmaceutical composition of any one of aspects 1 to 54, wherein the excipient granules have an average bulk density from 0.5 g/mL to 0.7 g/mL, wherein the bulk density is determined according to USP 616, Method 1.

Aspect 56. The pharmaceutical composition of any one of aspects 50 to 55, wherein the viscosifying agent comprises xanthan gum.

Aspect 57. The pharmaceutical composition of aspect 56, wherein the xanthan gum has an average particle size from 25 μm to 100 μm, wherein the particle size is determined using sieve analysis or laser diffraction.

Aspect 58. The pharmaceutical composition of aspect 56, wherein the xanthan gum has an average particle size of less than 75 μm, wherein the particle size is determined using sieve analysis or laser diffraction.

Aspect 59. The pharmaceutical composition of any one of aspects 56 to 58, wherein the xanthan gum has a viscosity ratio from 1.0 to 1.5.

Aspect 60. The pharmaceutical composition of any one of aspects 56 to 59, wherein the xanthan gum provides a viscosity from 1,200 mPa×sec to 1600 mPa×sec (cP).

Aspect 61. The pharmaceutical composition of any one of aspects 50 to 60, wherein the disintegrant comprises crospovidone.

Aspect 62. The excipient granulation of aspect 61, wherein the crospovidone has: a pH value from 5 to 8 as a 1% solution in water at 25° C.; a bulk density from 0.25 g/cm³ to 0.45 g/cm³ and/or an average particle size from 20 μm to 40 μm.

Aspect 63. The pharmaceutical composition of any one of aspects 50 to 62, wherein the one or more additional excipients comprises a diluent, a binder, a flavoring agent, a buffer, an acid, a sweetening agent, a filler, or a combination of any of the foregoing.

Aspect 64. The pharmaceutical composition of any one of aspects 50 to 63, wherein the one or more additional excipients comprises a binder.

Aspect 65. The pharmaceutical composition of aspect 64, wherein the binder comprises polyvinylpyrrolidone.

Aspect 66. The pharmaceutical composition of aspect 65, wherein the polyvinylpyrrolidone has: a K-value from 29 to 32; a number average molecular weight of 58,000 Daltons; a viscosity of 2.5 cP in a 5% aqueous solution at 25° C.; an average particle size of 80 μm; a bulk density of 0.36 g/cm³; and/or a tapped density of 0.47 g/cm³.

Aspect 67. The pharmaceutical composition of any one of aspects 64 to 66, wherein, the excipient granules comprise from 5 wt % to 20 wt % of the binder; and wt % is based on the total weight of the excipient granules.

Aspect 68. The pharmaceutical composition of any one of aspects 50 to 67, wherein the one or more additional excipients comprises a diluent.

Aspect 69. The pharmaceutical composition of aspect 68, wherein the diluent comprises anhydrous dibasic calcium phosphate.

Aspect 70. The pharmaceutical composition of any one of aspects 68 to 69, wherein, the excipient granules comprise from 10 wt % to 30 wt % of the diluent; and wt % is based on the total weight of the excipient granules.

Aspect 71. The pharmaceutical composition of any one of aspects 50 to 70, wherein the one or more additional excipients comprises a filler.

Aspect 72. The pharmaceutical composition of aspect 71, wherein the filler comprises microcrystalline cellulose.

Aspect 73. The pharmaceutical composition of any one of aspects 71 to 72, wherein, the excipient granules comprise from 1 wt % to 20 wt % of the binder; and wt % is based on the total weight of the excipient granules.

Aspect 74. The pharmaceutical composition of any one of aspects 50 to 73, wherein the one or more additional excipients comprises a flavoring agent.

Aspect 75. The pharmaceutical composition of aspect 74, wherein the flavoring agent comprises a citric fruit flavoring agent, a berry flavoring agent, a punch flavoring agent, or a combination of any of the foregoing.

Aspect 76. The pharmaceutical composition of any one of aspects 74 to 75, wherein, the excipient granules comprise from 15 wt % to 35 wt % of the flavoring agent; and wt % is based on the total weight of the excipient granules.

Aspect 77. The pharmaceutical composition of any one of aspects 50 to 76, wherein the one or more additional excipients comprises a buffer.

Aspect 78. The pharmaceutical composition of aspect 77, wherein the buffer comprises sodium citrate anhydrate.

Aspect 79. The pharmaceutical composition of any one of aspects 77 to 278, wherein, the excipient granules comprise from 0.5 wt % to 6.5 wt % of the buffer; and wt % is based on the total weight of the excipient granules.

Aspect 80. The pharmaceutical composition of any one of aspects 50 to 79, wherein the one or more additional excipients comprises an acid.

Aspect 81. The pharmaceutical composition of aspect 80, wherein the acid comprises citric acid anhydrous.

Aspect 82. The pharmaceutical composition of any one of aspects 80 to 81, wherein, the excipient granules comprise from 0.5 wt % to 8 wt % of the acid; and wt % is based on the total weight of the excipient granules.

Aspect 83. The pharmaceutical composition of any one of aspects 50 to 82, wherein the one or more additional excipients comprises a sweetening agent.

Aspect 84. The pharmaceutical composition of aspect 83, wherein the sweetening agent comprises sucralose.

Aspect 85. The pharmaceutical composition of any one of aspects 83 to 84, wherein, the excipient granules comprise from 0.1 wt % to 6 wt % of the sweetening agent; and wt % is based on the total weight of the excipient granules.

Aspect 86. The pharmaceutical composition of aspect 63 wherein the excipient granules comprise: from 2 wt % to 22 wt % of the binder; from 10 wt % to 30 wt % of the diluent; from 1 wt % to 20 wt % of the filler; from 15 wt % to 35 wt % of the flavoring agent; from 0.5 wt % to 6.5 wt % of the buffer; from 0.5 wt % to 8 wt % of the acid; and/or from 0.1 wt % to 6 wt % of the sweetening agent, wherein wt % is based on the total weight of the excipient granules.

Aspect 87. The pharmaceutical composition of aspect 86, wherein the excipient granules comprise: from 1 wt % to 35 wt % of the viscosifying agent; and from 1 wt % to 25 wt % of the disintegrant; wherein wt % is based on the total weight of the excipient granules.

Aspect 88. The pharmaceutical composition of any one of aspects 86 to 87, wherein the excipient granules comprise: from 5 wt % to 15 wt % of the binder; from 17 wt % to 27 wt % of the diluent; from 6 wt % to 16 wt % of the filler; from 20 wt % to 30 wt % of the flavoring agent; from 1 wt % to 5 wt % of the buffer; from 2 wt % to 6 wt % of the acid; and/or from 1 wt % to 5 wt % of the sweetening agent, wherein wt % is based on the total weight of the excipient granules.

Aspect 89. The pharmaceutical composition of aspect 88, wherein the excipient granules comprise: from 5 wt % to 15 wt % of the viscosifying agent; and from 7 wt % to 17 wt % of the disintegrant; wt % is based on the total weight of the excipient granules.

Aspect 90. The pharmaceutical composition of any one of aspects 88 to 89, wherein, the viscosifying agent comprises xanthan gum; the disintegrant comprises crospovidone; the binder comprises polyvinylpyrrolidone; the diluent comprises dicalcium phosphate; the filler comprises microcrystalline cellulose; the flavoring agent comprises a berry/punch flavoring agent; the buffer comprises sodium citrate dihydrate; the acid comprises citric acid anhydrous; and the sweetening agent comprises sucralose, wherein wt % is based on the total weight of the excipient granules.

Aspect 91. The pharmaceutical composition of any one of aspects any one of aspects 50 to 90, wherein, the excipient granules have an average particle size from 100 μm to 600 μm; and the particle size is determined by sieve analysis or laser diffraction.

Aspect 92. The pharmaceutical composition of any one of aspects 50 to 91, wherein, the excipient granules have a particle size distribution characterized by 8.5 wt % greater than 35 mesh (500 μm), 39 wt % from 35-60 mesh (500-250 μm), 52.2% pan, less than 35 mesh (500 μm) 52.2 wt %; and the particle size is determined by sieve analysis or laser diffraction.

Aspect 93. The pharmaceutical composition of any one of aspects 1 to 92, wherein, the excipient granulation (35-60 mesh fraction) has an average bulk density from 0.5 g/mL to 0.9 g/mL; and the bulk density is determined using a bulk density cylinder.

Aspect 94. The pharmaceutical composition of any one of aspects 1 to 93, wherein, the excipient granulation has an average bulk density from 0.6 g/mL to 0.7 g/mL (35-60 mesh fraction); and the bulk density is determined using a bulk density cylinder.

Aspect 95. The pharmaceutical composition of any one of aspects 1 to 94, wherein the excipient granulation, when reconstituted in water at 25° C., completely dissolves in less than 60 seconds.

Aspect 96. The pharmaceutical composition of any one of aspects 1 to 95, wherein the excipient granulation, when reconstituted in in water at 25° C., completely dissolves in less than 30 seconds.

Aspect 97. The pharmaceutical composition of any one of aspects 1 to 96, wherein the excipient granulation, when reconstituted as a 1 w/v % solution in water at 25° C., completely dissolves in less than 60 seconds.

Aspect 98. The pharmaceutical composition of any one of aspects 1 to 97, wherein the excipient granulation, when reconstituted as a 1 w/v % solution in water at 25° C., completely dissolves in less than 30 seconds.

Aspect 99. The pharmaceutical composition of any one of aspects 1 to 98, wherein the excipient granulation has an average Hausner ratio from 1.1 to 1.25, wherein the Hausner ratio is determined according to USP <1174>.

Aspect 100. The pharmaceutical granulation of any one of aspects 1 to 99, wherein the excipient granulation has an average compressibility index from 12 to 18.

Aspect 101. The pharmaceutical composition of any one of aspects 1 to 100, wherein the excipient granulation has an average Flodex value of less than 5 mm.

Aspect 102. The pharmaceutical composition of any one of aspects 1 to 101, wherein, the excipient granulation has an average friability of less than 2 wt %; and the friability is determined using a sonic sifter.

Aspect 103. The pharmaceutical composition of any one of aspects 1 to 102, wherein the excipient granulation provides a pH from 4.0 to 8.0 when reconstituted as a 1 w/v % solution of water.

Aspect 104. The pharmaceutical composition of any one of aspects 1 to 103, wherein, when 1.2 g of the excipient granulation is reconstituted in 40 mL of water at 25° C., the resulting solution exhibits a viscosity vs. time profile as shown in FIG. 2.

Aspect 105. The pharmaceutical composition of any one of aspects 1 to 104, wherein, when 1.2 g of the excipient granulation is reconstituted in 40 mL of water at 25° C., the resulting solution exhibits a luminosity vs. time profile as shown in FIG. 3.

Aspect 106. The pharmaceutical composition of any one of aspects 1 to 105, wherein after mixing a solution of 1.2 g of the excipient granulation and 40 mL of water at 25° C. for 90 seconds, the solution has a viscosity within a range from 45 cP to 80 cP.

Aspect 107. The pharmaceutical composition of any one of aspects 1 to 106, wherein after mixing a solution of 1.2 g of the excipient granulation and 40 mL of water at 25° C. for 30 seconds, the solution has a viscosity within a range from 15 cP to 40 cP.

Aspect 108. The pharmaceutical composition of any one of aspects 1 to 107, wherein after mixing a solution of 1.2 g of the excipient granulation and 40 mL of water at 25° C. for 60 seconds, the solution has a viscosity within a range from 35 cP to 60 cP.

Aspect 109. The pharmaceutical composition of any one of aspects 50 to 108, wherein the excipient granules have a dissolution profile characterized by complete dissolution within 30 seconds.

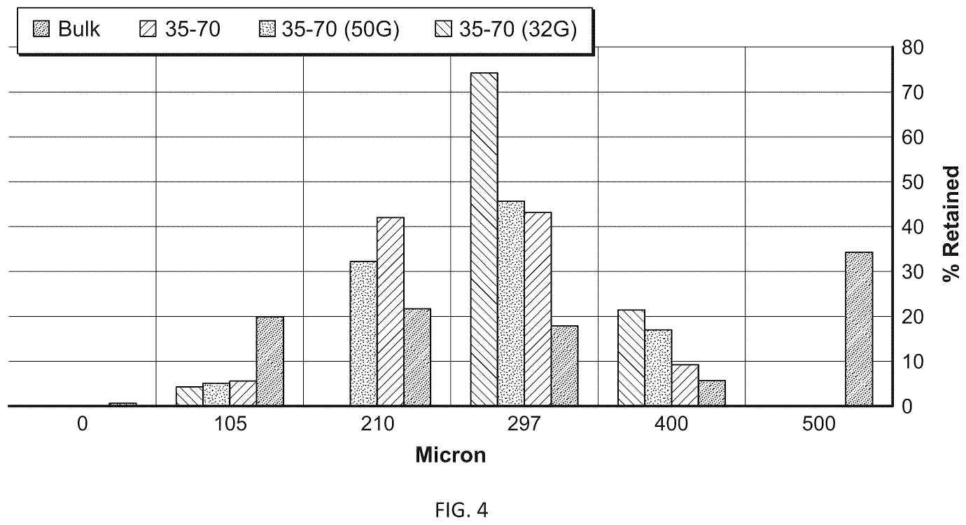

Aspect 110. The pharmaceutical composition of any one of aspects 50 to 109, wherein the excipient granules have a dissolution profile as shown in FIG. 4.

Aspect 111. The pharmaceutical composition of any one of aspects 50 to 1110, wherein the excipient granules have a reconstitution viscosity characterized by a viscosity from 15 cP to 35 cP within 5 seconds and from 40 cP to 60 cP within 60 seconds.

Aspect 112. The pharmaceutical composition of any one of aspects 50 to 111, wherein the excipient granules have a reconstitution viscosity as shown in FIG. 5.

Aspect 113. The pharmaceutical composition of any one of aspects 50 to 112, wherein the excipient granules have a reconstitution luminosity characterized by a luminosity from 70 to 75 within 30 seconds.

Aspect 114. The pharmaceutical composition of any one of aspects 50 to 113, wherein the excipient granules have a reconstitution viscosity as shown in FIG. 6.

Aspect 115. The pharmaceutical composition of any one of aspects 1 to 114, wherein, the excipient granulation has a first bulk density; the pharmaceutical granulation has a second bulk density; and the first bulk density is within +/−20% of the second bulk density.

Aspect 116. The pharmaceutical composition of any one of aspects 1 to 115, wherein, the excipient granulation has a first average particle size; the pharmaceutical granulation has a second average particle size; and the first average particle size is within +/−20% of the second average particle size.

Aspect 117. The pharmaceutical composition of any one of aspects 1 to 116, wherein, the excipient granulation has a first tapped density; the pharmaceutical granulation has a second tapped; and the first average tapped density is within +/−20% of the tapped density.

Aspect 118. The pharmaceutical composition of any one of aspects 1 to 117, wherein, the excipient granulation has a first Hausner ratio; the pharmaceutical granulation has a second Hausner ratio; and the first Hausner ratio is within +/−20% of the second Hausner ratio.

Aspect 119. The pharmaceutical composition of any one of aspects 1 to 118, wherein, the excipient granulation has a first compressibility index; the pharmaceutical granulation has a second compressibility index; and the first compressibility index is within +/−20% of the second compressibility index.

Aspect 120. The pharmaceutical composition of any one of aspects 1 to 119, wherein, the excipient granulation has a first Flodex value; the pharmaceutical granulation has a second Flodex value; and the first Flodex value is within +/−20% of the second Flodex value.

Aspect 121. The pharmaceutical composition of any one of aspects 1 to 120, wherein, the excipient granulation has a first friability value; the pharmaceutical granulation has a second friability value; and the first friability value is within +/−20% of the second friability value.

Aspect 122. The pharmaceutical composition of 1 to 121, wherein the pharmaceutical composition comprises one or more additional excipients.

Aspect 123. The pharmaceutical composition of aspect 122, wherein the one or more additional excipients is in the form of a powder.

Aspect 124. The pharmaceutical composition of any one of aspects 122 to 123, wherein, the pharmaceutical composition comprises less than 5 wt % of the one or more additional excipients; and wt % is based on the total weight of the pharmaceutical composition.

Aspect 125. The pharmaceutical composition of any one of aspects 122 to 124, wherein the one or more additional excipients comprises an antistatic agent.

Aspect 126. The pharmaceutical composition of aspect 125, wherein the antistatic agent comprises hydrous magnesium silicate.

Aspect 127. The pharmaceutical composition of any one of aspects 125 to 126, wherein, the pharmaceutical composition comprises from 0.1 wt % to 1.0 wt % of the antistatic agent; and wt % is based on the total weight of the pharmaceutical composition.

Aspect 128. A pharmaceutical product comprising the pharmaceutical composition of any one of aspects 1 to 127.

Aspect 129. The pharmaceutical product of aspect 128, wherein the pharmaceutical product comprises the pharmaceutical composition contained within a package.

Aspect 130. The pharmaceutical product of aspect 129, wherein the package comprises a sachet.

Aspect 131. The pharmaceutical product of any one of aspects 129 to 130, wherein the pharmaceutical product comprises the excipient granulation and a pharmaceutical granulation combined in the same package.

Aspect 132. The pharmaceutical product of any one of aspects 129 to 121, wherein the pharmaceutical product comprises; a first package containing the excipient granulation; and a second package containing the pharmaceutical granulation.

Aspect 133. The product of aspect 132, wherein the first package and the second package comprise a sachet.

Aspect 134. The pharmaceutical product of aspect 128, wherein the pharmaceutical product comprises; a first package containing the excipient granulation; a second package containing the immediate release pharmaceutical granulation; and a third package containing a modified release pharmaceutical granulation.

Aspect 135. The pharmaceutical product of aspect 134, wherein each of the first package, the second package and the third package is a sachet or a stick pack.

Aspect 136. The pharmaceutical product of aspect 128, wherein the pharmaceutical product comprises: a first compartment containing the excipient granulation; and a second compartment containing the pharmaceutical granulation.

Aspect 137. The pharmaceutical product of aspect 128, wherein the pharmaceutical product comprises: a first compartment containing the excipient granulation; a second compartment containing the immediate release pharmaceutical granulation; and a third compartment containing the modified release pharmaceutical granulation.

Aspect 138. The pharmaceutical product of any one of aspects 128 to 137, wherein the pharmaceutical product is configured to combine the excipient granulation with the pharmaceutical granulation during use to provide a pharmaceutical composition comprising the excipient granulation and the pharmaceutical granulation.

Aspect 139. The pharmaceutical product of any one of aspects 128 to 138, wherein the pharmaceutical product comprises from 0.5 g to 25 g of the pharmaceutical composition.

Aspect 140. The pharmaceutical product of any one of aspects 128 to 139, wherein the pharmaceutical product comprises a therapeutically effective dose of 4-((L-valyl)oxy)butanoic acid.

Aspect 141. The product of any one of aspects 128 to 140, wherein the pharmaceutical product comprises from 2 g to 20 of 4-((L-valyl)oxy)butanoic acid.

Aspect 142. A unit dose comprising the pharmaceutical composition of any one of aspects 1 to 127.

Aspect 143. The unit dose of aspect 142, wherein the unit dose comprises from 2 g to 20 g of 4-((L-valyl)oxy)butanoic acid.

Aspect 144. A partial unit dose comprising the pharmaceutical composition of any one of aspects 1 to 127.

Aspect 145. The partial unit dose of aspect 144, wherein the partial unit dose comprises from 2 g to 20 g of 4-((L-valyl)oxy)butanoic acid.

Aspect 146. A kit comprising: the pharmaceutical composition of any one of aspects any one of aspects 1 to 127; the pharmaceutical product of any one of aspects 128 to 141; and instructions for using contents of the kit.

Aspect 147. A method of using the pharmaceutical composition of any one of aspects any one of aspects 1 to 127 or the pharmaceutical product of any one of aspects 128 to 141 to prepare an oral pharmaceutical composition for oral administration comprising: combining the pharmaceutical composition with an aqueous solution; and mixing the pharmaceutical composition to dissolve the immediate release pharmaceutical granulation and the excipient granulation to form the viscous oral pharmaceutical composition.

Aspect 148. The method of aspect 147, wherein the aqueous solution is water.

Aspect 149. The method of aspect 417, wherein the aqueous solution is tap water at a temperature from 20° C. to 25° C.

Aspect 150. The method of any one of aspects 147 to 149, wherein the viscous oral pharmaceutical composition has a viscosity from 50 cP to 1,000 cP at a temperature from 23° C., wherein the viscosity is determined using a Brookfield Digital Viscometer Model LVDV-I+ equipped with a Brookfield SC4-18 spindle at a spindle speed of 30 rpm.

Aspect 151. The method of any one of aspects 147 to 150, wherein the method comprises combining the pharmaceutical composition and the aqueous solution in a weight to volume ratio of from 0.25 to 0.74, wherein the ratio is the weight of the pharmaceutical composition in g divide by the volume of the aqueous solution in mL.

Aspect 152. The method of any one of aspects 147 to 151, wherein the method comprises combining the aqueous solution to the pharmaceutical composition in a volume/weight ratio of from 1.5 to 2.5, wherein the ratio refers to the volume of the aqueous solution in mL divided by the weight of the pharmaceutical composition in grams.

Aspect 153. The method of any one of aspects 147 to 152, wherein the modified release pharmaceutical granulation is suspended in the viscous solution.

Aspect 154. An oral pharmaceutical composition prepared by combining and mixing the pharmaceutical composition of any one of aspects 1 to 127 with water.

Aspect 155. The oral pharmaceutical composition of aspect 154, wherein combining comprising combining from 10 g to 25 g of the pharmaceutical composition with from 30 mL to 50 mL of water.

Aspect 156. The oral pharmaceutical composition of any one of aspects 154 to 155, wherein the oral pharmaceutical composition comprises: a viscous aqueous solution; a modified release granulation suspended in the viscous aqueous solution.

Aspect 157. The oral pharmaceutical composition of aspect 156, wherein the viscous aqueous solution has a viscosity from 50 cP to 1,000 cP at a temperature from 20° C. to 25° C. such as 23° C., wherein the viscosity is determined by combining 3,164 mg of the pharmaceutical composition is added to 40 mL of water and mixing using a Brookfield Digital Viscometer Model LVDV-I+ equipped with a Brookfield SC4-18 spindle at a spindle speed of 30 rpm.

Aspect 158. The oral pharmaceutical composition of any one of aspects 156 to 157, wherein the viscous solution comprises dissolved 4-((L-valyl)oxy)butanoic acid.

Aspect 159. The oral pharmaceutical composition of any one of aspects 154 to 158, wherein the oral pharmaceutical composition has a volume from 30 mL to 60 mL.

Aspect 160. The oral pharmaceutical composition of aspect 154, wherein the oral pharmaceutical composition comprises a viscous solution comprising dissolved 4-((L-valyl)oxy)butanoic acid and a modified release granulation comprising 4-((L-valyl)oxy)butanoic acid suspended in the viscous solution.

Aspect 161. The oral pharmaceutical composition of aspect 160, wherein, from 17 wt % to 37 wt % of the 4-((L-valyl)oxy)butanoic acid is dissolved in the viscous solution; the modified release pharmaceutical granulation comprises 63 wt % to 83 wt % of the 4-((L-valyl)oxy)butanoic acid; and wt % is based on the total weight of 4-((L-valyl)oxy)butanoic acid in the oral pharmaceutical composition.

Aspect 162. The oral pharmaceutical composition of any one of aspects 154 to 160, wherein the oral pharmaceutical composition comprises a therapeutically effective dose of 4-((L-valyl)oxy)butanoic acid.

Aspect 163. The oral pharmaceutical composition of any one of aspects 154 to 161, wherein the oral pharmaceutical composition comprises a unit dose of 4-((L-valyl)oxy)butanoic acid.

Aspect 164. The oral pharmaceutical composition of any one of aspects 156 to 163, wherein the viscous solution has a volume of about 40 mL.

Aspect 165. The oral pharmaceutical composition of any one of aspects 156 to 163, wherein the viscous solution has a volume from 20 mL to 60 mL.

Aspect 166. The oral pharmaceutical composition of any one of aspects 156 to 165, wherein the viscous oral solution comprises from 10 g to 20 g of 4-((L-valyl)oxy)butanoic acid.

Aspect 167. A method of treating a disease in a patient comprising orally administering to a patient in need of such treatment a therapeutically effective amount of the oral pharmaceutical composition of any one of aspects 154 to 166.

Aspect 168. The method of aspect 167, wherein the disease is capable of being treated with γ-hydroxybutyric acid.

Aspect 169. The method of aspect 167, wherein the disease is selected from narcolepsy, cataplexy, cataplexy with narcolepsy, excessive daytime sleepiness, a sleep disorder associated with Parkinson's disease, Parkinson's disease, a neurodegenerative disease, sleep disturbance syndrome, fatigue, improving nocturnal sleep, hypnagogic hallucinations, sleep paralysis, fragmented sleep, alcohol withdrawal and dependence, obstructive sleep apnea syndrome, insomnia, insomnia associated with schizophrenia, sleep ignition and maintenance disorders, chronic fatigue syndrome, essential tremor, hemiplegia in patients with alternating hemiplegia of childhood, sedative abuse, and binge eating disorder.

Aspect 170. The method of aspect 167, wherein the disease is fatigue or excessive daytime sleepiness associated with narcolepsy.

Aspect 171. The method of aspect 167, wherein the disease is narcolepsy, excessive daytime sleepiness, cataplexy, excessive daytime sleepiness associated with narcolepsy, excessive daytime sleepiness associated with Parkinson's disease, excessive daytime sleepiness associated with multiple sclerosis, cataplexy associated with narcolepsy, fatigue, fatigue associated with Parkinson's diseases, fatigue associated with multiple sclerosis, or fibromyalgia.

Aspect 172. The method of aspect 167, wherein the disease is cataplexy associated with narcolepsy.

Aspect 173. The method of aspect 167, wherein the disease is excessive daytime sleepiness associated with narcolepsy.

Aspect 174. The method of aspect 167, wherein the disease is excessive daytime sleepiness in a patient with Parkinson's disease.

Aspect 175. The method of aspect 167, wherein the disease is chronic fatigue in a patient with Parkinson's disease.

Aspect 176. A method of treating a symptom associated with narcolepsy, excessive daytime sleepiness, cataplexy, excessive daytime sleepiness associated with narcolepsy, excessive daytime sleepiness associated with Parkinson's disease, excessive daytime sleepiness associated with multiple sclerosis, cataplexy associated with narcolepsy, fatigue, fatigue associated with Parkinson's diseases, fatigue associated with multiple sclerosis, or fibromyalgia comprising orally administering to a patient in need of such treatment a therapeutically effective amount of the oral pharmaceutical composition of any one of aspects 154 to 166.

Aspect 177. A method of treating an REM sleep behavior disorder, spasmodic dystonia, schizophrenia, insomnia, insomnia associated with schizophrenia, idiopathic hypersomnia, chronic fatigue syndrome, cluster headache, Alzheimer's disease, essential tremor, post-traumatic stress syndrome, insomnia associated with post-traumatic stress syndrome, or anxiety comprising orally administering to a patient in need of such treatment a therapeutically effective amount of the oral pharmaceutical composition of any one of aspects 154 to 166.

Aspect 178. A method of treating a symptom associated with REM sleep behavior disorder, spasmodic dystonia, schizophrenia, insomnia, insomnia associated with schizophrenia, idiopathic hypersomnia, chronic fatigue syndrome, cluster headache, Alzheimer's disease, essential tremor, post-traumatic stress syndrome, insomnia associated with post-traumatic stress syndrome, or anxiety comprising orally administering to a patient in need of such treatment a therapeutically effective amount of the oral pharmaceutical composition of any one of aspects 154 to 166.

Aspect 179. The method of any one of aspects 167 to 178, wherein administering comprises administering QD.

Aspect 180. The method of any one of aspects 167 to 178, wherein administering comprises administering BID.

Aspect 181. The pharmaceutical composition of any one of aspects 1 to 127, wherein following storage at 5±3° C. or at 25±2° C./60±5% RH in a sealed high barrier pouch for 18 months, the 4-((L-valyl)oxy)butanoic acid dissolution profile is similar to the 4-((L-valyl)oxy)butanoic acid dissolution profile prior to storage.

Aspect 182. The pharmaceutical composition of any one of aspects 1 to 127, wherein following storage of the pharmaceutical composition provided by the present disclosure at 5±3° C. or at 25±2° C./60±5% RH sealed in high barrier pouch for 18 months, the reconstitution time is about 15 seconds.

Aspect 183. The pharmaceutical composition of any one of aspects 1 to 127, wherein following storage at 5±3° C. or at 25±2° C./60±5% RH in a sealed high barrier pouch for 18 months, the 4-((L-valyl)oxy)butanoic acid content is greater than 96% of the 4-((L-valyl)oxy)butanoic acid prior to storage.

Aspect 184. The pharmaceutical composition of any one of aspects 1 to 127, wherein following storage at 5±3° C. in a sealed in high barrier pouch for 18 months, the total impurity content is 0.3% or less and following storage at or at 25±2° C./60±5% RH in a sealed high barrier pouch for 18 months, the total impurity content is 0.6% or less.

Aspect 185. The pharmaceutical composition of any one of claims 1 to 127, wherein following storage at 5±3° C. in a sealed high barrier pouch for 18 months, the L-valine impurity content is 0.3% or less and following storage at or at 25±2° C./60±5% RH in a sealed high barrier pouch for 18 months, the L-valine impurity content is 0.4% or less.

### EXAMPLES

Embodiments provided by the present disclosure are further illustrated by reference to the following examples, which describe pharmaceutical granulations, excipient granulations, and pharmaceutical compositions. It will be apparent to those skilled in the art that many modifications, both to materials, and methods, may be practiced without departing from the scope of the disclosure. It should be noted that there are alternative ways of implementing the embodiments disclosed herein. Accordingly, the present embodiments are to be considered as illustrative and not restrictive. Furthermore, the claims are not to be limited to the details given herein and are entitled their full scope and equivalents thereof.

### General Methods

Plasma concentrations of 4-((L-valyl)oxy)butanoic acid and γ-hydroxybutyric acid in plasma of healthy human subjects were measured using liquid chromatography tandem mass-spectroscopy and evaluated using PHOENIX™ WINNONLIN® version 8.1 (Pharsight Corporation, USA) and MICROSOFT® EXCEL® 2016 (Microsoft Corporation, USA).

### Example 1

### Immediate Release Pharmaceutical Granulation

An immediate release pharmaceutical having the constitutes listed in Table 1 was prepared.

Immediate release pharmaceutical granules comprising 4-((L-valyl)oxy)butanoic acid were prepared using MicroPX® micro-pelletizing technology (Glatt GmbH). The immediate release pharmaceutical granules had an average granule diameter (D50) from 225 μm to 275 μm. The immediate release pharmaceutical granules contained 90 wt % 4-((L-valyl)oxy)butanoic acid, 5 wt % USP magnesium silicate, and 5 wt % hypromellose (hydroxypropylmethyl cellulose), where wt % is based on the total weight of the immediate release pharmaceutical granules.

TABLE 1
Immediate Release Pharmaceutical Granulation.

| Component | % w/w |
|---|---|
| 4-((L-Valyl)oxy)butanoic acid | 90.0 |
| Hydroxypropylmethyl cellulose | 5.0 |
| Talc | 5.0 |
| Total | 100.0 |

A continuous fluid bed pelletizing process involving a combination of layering, coating and partial spray drying was used to prepare the immediate release granulation.

The yield in the target particle size range of from 200 μm to 500 μm was greater than 90%.

A dissolution profile for the immediate release pharmaceutical granulation was determined using a USP Type 2 dissolution apparatus with a buffered solution at pH 4.5 at a temperature of 37° C. and a paddle speed of 75 rpm. A dissolution profile for the release of 4-((L-valyl)oxy)butanoic acid from the immediate pharmaceutical granulation is shown in FIG. 1.

### Example 2

### Modified Release Pharmaceutical Granulation

A modified release pharmaceutical granulation having the constituents listed in Table 2 was prepared.

TABLE 2
Modified Release Granulation Composition.

| Component | % w/w |
|---|---|
| Immediate Release Granules of | 83.24 |
| Example 3 | |
| Ethylcellulose | 12.23 |
| Hydroxypropyl cellulose | 0.69 |
| Dibutyl Sebacate | 1.24 |
| Talc | 2.61 |
| Total | 100.00 |

The modified release granulation was prepared using Wurster Fluid Bed coating. The immediate release granules of Example 1 were coated in a fluid bed equipped with a Wurster column. The coating included 73.0 wt %, ethylcellulose, 4.1 wt % hydroxypropylcellulose, 7.4 wt % dibutyl sebacate, and 15.6 wt % talc, where wt % is based on the total weight of the coating. See Table 2. Because the coating did not include pH dependent release polymers, the release profile of the modified release pharmaceutical granulation is not impacted by food, gastric emptying times or stomach pH. The coating was applied to provide a weight gain of 20% wg.

The dissolution profile for the modified release pharmaceutical granulation was determined using a USP Type 2 dissolution apparatus with a buffered solution at pH 4.5 at a temperature of 37° C. and a paddle speed of 75 rpm. The dissolution profile for the release of 4-((L-valyl)oxy)butanoic acid from the modified release pharmaceutical granulation is shown in FIG. 2.

The dissolution profiles were measured using an Agilent dissolution bath and autosampler equipped with Apparatus II paddles. The dissolution media was made by combining and mixing 2.99 g of sodium acetate trihydrate, 1.2 mL of glacial acetic acid, and 1,000 mL of water, with the pH adjusted to 4.5±0.05 using acetic acid. Five-hundred (500) mL of the dissolution media was transferred to a vessel and the temperature equilibrated to 37° C.±0.5° C. A pharmaceutical granulation comprising the immediate release granulation and the modified release granulation (3,164 mg) was added with the paddles spinning at 75 rpm. Samples were taken at intervals and the content of 4-((L-valyl)oxy)butanoic acid measured using HPLC.

A dissolution profile for the pharmaceutical granulation containing the immediate release (Example 1) and the modified release granulation was determined using a USP Type 2 dissolution apparatus with a buffered solution at pH 4.5 at a temperature of 37° C. and a paddle speed of 75 rpm. The pharmaceutical granulation included about 73 wt % of the modified release granules and about 27 wt % of the immediate release granules. The dissolution profile for the release of 4-((L-valyl)oxy)butanoic acid from the pharmaceutical granulation is shown in FIG. 3.

### Example 3

### Excipient Granulation

An excipient granulation having the constituents listed in Table 3 was prepared.

TABLE 3
Excipient Granulation Composition.

| Component | % w/w |
|---|---|
| Xanthan Gum | 10.0 |
| Dicalcium Phosphate | 21.5 |
| Microcrystalline Cellulose | 10.8 |
| Flavoring Agent | 25.0 |
| Crospovidone | 12.0 |
| Polyvinylpyrrolidone | 10.0 |
| Sodium Citrate Dihydrate | 3.3 |
| Citric Acid Anhydrous | 4.2 |
| Sucralose | 3.2 |
| Total | 100.0 |

An excipient granulation was produced using high shear granulation. The pharmaceutically acceptable excipients were selected to optimize the administration and palatability of the 4-((L-valyl)oxy)butanoic acid-containing oral pharmaceutical composition.

The methods to produce excipient granules were (1) dry granulation through roller compaction and/or slugging on a tablet press, and (2) high-shear wet granulation using either a small coffee grinder for small scale experiments or on a 4 L bowl high-shear granulator for the larger batch size.

Excipient granules were prepared by dry mixing the constituents and then granulating the dry mixture with a buffer comprising the sodium citrate dihydrate and the citric acid anhydrous dissolved in water to provide a first granulation. The first granulation was wet massed to provide a first wet mass, which was then granulated with water to provide a second granulation. The second granulation was wet massed to provide a second wet mass and dried to provide the excipient granules.

A Collette (GEA) GRAL 25 high shear granulator equipped with a 25 L jacked bowl was used for granulation. The jacketed bowl was coupled to an Advantage Engineering Air cooled water chiller set at 46° F. (8° C.). Buffer or water was sprayed into the mixture using a two fluid nozzle with a 1-mm nozzle orifice at an atomization pressure of 45 bar.

The excipient granules were dried using a Freund-Vector FLM-3 fluid bed drier at 60° C.

The dried excipient granules were milled using a Quadro Comil®197 equipped with 50G or 32G screens and sieved using a tabletop vibratory sieving base equipped with #35 and #70 mesh screens. Fines (<210 μm) passing through the #70 mesh screen were removed.

FIG. 4 shows the particle size distribution of the excipient granules prepared at different stages in the sieving process. The size distributions include the distributions of the bulk granules before sieving (bulk), the 35G-75G screen fraction (35-70), after passing through the 50G screen (50G), after passing through the 32G screen (30G). The particle size distribution was determined using sieve analysis or laser diffraction.

The excipient granules (1.2 g) were combined and mixed with 40 mL of water to provide a reconstituted viscous solution. The pH of the reconstituted viscous solution was 4.3.

The reconstitution viscosity profile for the excipient granules is shown in FIG. 5. The reconstitution viscosity was determined using a Brookfield Digital Viscometer Model LVDV-I+ fitted with a Brookfield SC4-18 spindle at a speed of 30 rpm. Excipient granules (1.2 g) were added to 40 mL of water at 23° C.

The reconstitution luminosity profile for the excipient granulation is shown in FIG. 6 for the same solution used to measure the reconstitution viscosity.

The excipient granules had a bulk density from 0.58 g/mL to 0.62 g/mL, a tapped density from 0.69 g/mL to 0.74 g/mL, a Hausner Ratio from 0.18 to 1.20, a compressibility index from 15.5 to 16.8, and a Flodex value of 4.

### Example 4

### Oral Pharmaceutical Composition

An oral pharmaceutical composition was prepared by combining the immediate granulation of Example 1, the modified release pharmaceutical granulation of Example 2, and the excipient granulation of Example 3.

Table 4 shows the relative amounts of the immediate release granulation and the modified release granulation in terms of % w/w of the pharmaceutical granulation; the amount of 4-((L-valyl)oxy)butanoic acid in the immediate release and modified release granulations, respectively; and the % w/w of 4-((L-valyl)oxy)butanoic acid in the immediate release and modified release granulations based on the total weight of 4-((L-valyl)oxy)butanoic acid in the pharmaceutical composition.

TABLE 4
Pharmaceutical granulation.

| | 4-((L- | % w/w | |
|---|---|---|---|
| | valyl)oxy)butanoic | 4-((L- | |
| | acid | valyl)oxy)butanoic | % |
| Component | Amount | acid | w/w |
| Immediate release | 90% | 31% | 27% |
| granulation | | | |
| Modified release | 75% | 69% | 73% |
| granulation | | | |
| Total | 79% | 100% | 100% |

Table 5 shows the amounts of the constituents of the oral pharmaceutical composition comprising a 14.5 g dose of 4-((L-valyl)oxy)butanoic acid. The dose refers to the dose of 4-((L-valyl)oxy)butanoic acid.

TABLE 5
Pharmaceutical composition comprising 14.5
g of 4-((L-valyl)oxy)butanoic acid.

| | Dose | Dose | Weight | Weight |
|---|---|---|---|---|
| Component | (g) | (%) | (g) | (w/w %) |
| Modified release granulation | 10.0 | 69.0 | 13.34 | 66.89 |
| Immediate release granulation | 4.5 | 31.0 | 5.00 | 25.08 |
| Excipient granulation | — | — | 1.50 | 7.53 |
| Talc | — | — | 0.10 | 0.50 |
| Total | 14.5 | 100 | 19.94 | 100 |

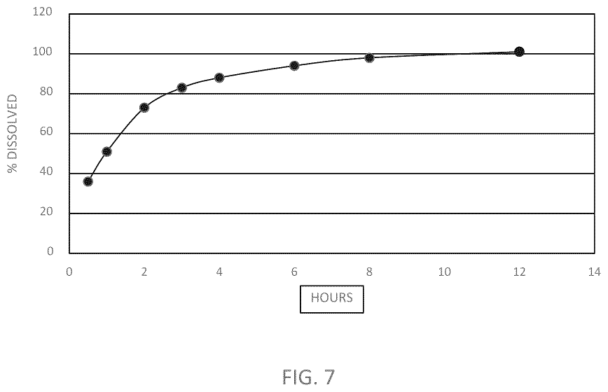

A dissolution profile for a pharmaceutical composition containing the excipient, the immediate release, and the modified release granulations was determined using a USP Type 2 dissolution apparatus with a buffered solution at pH 4.5 at a temperature of 37° C. and a paddle speed of 75 rpm. The pharmaceutical composition contained about 67 wt % of the modified release granulation, about 25.2 wt % of the immediate release pharmaceutical granulation, and about 7.6 wt % of the excipient granulation. A sodium acetate buffer solution was used as the dissolution media and contained 3,164 mg of the pharmaceutical composition in 500 mL of the dissolution media. The dissolution profile is shown in FIG. 7.

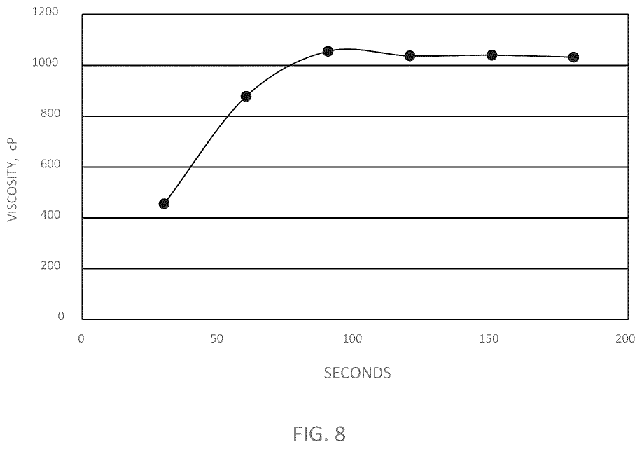

The reconstitution viscosity of the pharmaceutical compositions was determined by combining and mixing 14.5 g of the stored pharmaceutical composition with 40 mL of water at 23° C. and measuring the viscosity according to USP <912>, Method 1c using a No. 62 spindle at a speed of 12 rpm. The results are presented in FIG. 8.

### Example 5

### Pharmacokinetics of a Pharmaceutical Granulation

The immediate release pharmaceutical granules of Example 1 and the modified release pharmaceutical granules of Example 2 were combined and orally administered with water to twelve (12) fasted, healthy subjects. To prepare the combined release oral pharmaceutical compositions, the immediate release pharmaceutical granulation was dissolved in 30 mL of water. Modified release pharmaceutical granules were then added and swirled gently. Thirty (30) mL of Ora-Plus®, an oral suspending vehicle, was added and the suspension gently stirred. The subjects then drank the suspension. The subjects repeatedly rinsed the cup with up to 250 mL of water and drank the solution.

A description of the oral pharmaceutical formulations administered is provided in Table 6.

TABLE 6
Content of combined release granulations

| Immediate Release | Modified Release | Dose 4-((L- |
|---|---|---|
| Granulation | Granulation | valyl)oxy)butanoic acid |
| (g) | (g) | (g) |
| 1.8 | 4 | 5.8 |
| 2.7 | 6 | 8.7 |
| 3.6 | 8 | 11.6 |
| 4.5 | 10 | 14.5 |

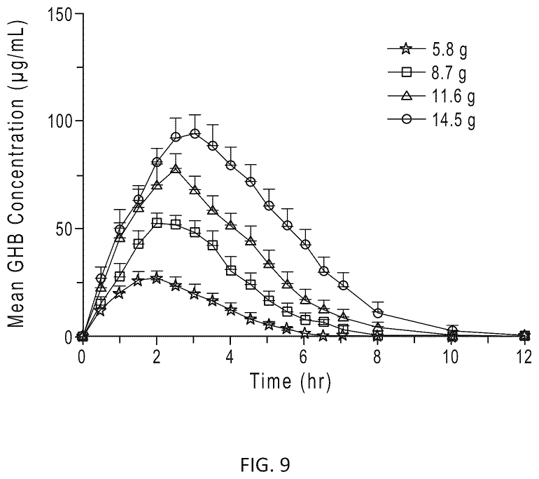

Certain of the plasma γ-hydroxybutyric acid pharmacokinetic parameters are provided in Table 7 and the corresponding plasma γ-hydroxybutyric acid pharmacokinetic profiles are shown in FIG. 9.

TABLE 7
Pharmacokinetic parameters for a pharmaceutical granulation.

| | | | Tₘₐₓ | Cₘₐₓ | AUC₀₋₆ | AUC₀₋₁₂ | AUCₗₐₛₜ | AUC_{inf} | CL/F | V/F | t_{1/2} |
|---|---|---|---|---|---|---|---|---|---|---|---|
| (Dose) | | | hr | hr × μg/mL | hr × μg/mL | hr × μg/mL | hr × μg/mL | hr × μg/mL | L/hr | L | hr |
| 5.8 | g | Mean | 1.7 | 31 | 88 | 90 | 89 | 90 | 81 | 91 | 0.8 |
| | | CV % | 33 | 35 | 52 | 53 | 53 | 52 | 45 | 51 | 23 |
| 8.7 | g | Mean | 2.3 | 61 | 185 | 194 | 193 | 195 | 52 | 79 | 0.9 |
| | | CV % | 31 | 29 | 38 | 43 | 43 | 42 | 40 | 114 | 82 |
| 11.6 | g | Mean | 2.4 | 81 | 285 | 311 | 310 | 311 | 44 | 44 | 0.7 |
| | | CV % | 16 | 31 | 38 | 44 | 44 | 44 | 43 | 46 | 20 |
| 14.5 | g | Mean | 2.6 | 104 | 389 | 455 | 454 | 457 | 37 | 47 | 0.8 |
| | | CV % | 32 | 24 | 33 | 41 | 42 | 42 | 38 | 47 | 44 |

### Example 6

### Storage Stability

The storage stability of combined immediate release and modified release pharmaceutical granulations and separately pharmaceutical compositions comprising an excipient granulation and a pharmaceutical granulation was determined.

A pharmaceutical granulation was prepared by combining an immediate release (IR) granulation of Example 1 (about 27 wt %) and the modified release (MR) granulation of Example 2 (about 73 wt %), were wt % is based on the total weight of the pharmaceutical granulation. Samples (3.61 g) of a combination of the immediate release pharmaceutical granules (Example 1) and modified release pharmaceutical granules (Example 2) were prepared and placed in a high barrier pouch (Amcor RFA-045, Specification No. 91038).

Pouches containing the pharmaceutical composition were (1) refrigerated at 5±3° C., (2) stored at 25±2° C./60±5% RH, or (3) under accelerated conditions at 40±2° C./75±5% RH, for up to 24 months. The 4-((L-valyl)oxy)butanoic acid content after storage is shown in Table 8.

TABLE 8
4-((L-Valyl)oxy)butanoic acid content of IR/MR
pharmaceutical granulations following storage.

| | | Long Term | Accelerated |
|---|---|---|---|
| Time | Refrigerated | 25 ± 2° C./ | 40 ± 2° C./ |
| (months) | 5 ± 3° C. | 60 ± 5% RH | 75 ± 5% RH |
| Initial | 102.0% | | |
| 0.5 | ¹ — | — | 102.7% |
| 1 | 102.2% | 103.7% | 102.0% |
| 3 | 98.8% | 98.0% | 101.9% |
| 6 | 102.1% | 102.4% | 101.7% |
| 9 | — | 101.8% | — |
| 12 | 102.3% | 101.7% | — |
| 18 | — | — | — |
| 24 | 102.6% | 102.2% | — |

¹ Not measured.

The amount of L-valine and total impurities in the samples is shown in Table 9.

TABLE 9
Impurity content of IR/MR pharmaceutical
granulations following storage.

| | | Long Term | Accelerated |
|---|---|---|---|
| Time | Refrigerated | 25 ± 2° C./ | 40 ± 2° C./ |
| (months) | 5 ± 3° C. | 60 ± 5% RH | 75 ± 5% RH |
| Initial | L-Valine: 0.10% | | |
| | Total: 0.1% | | |
| 0.5 | L-Valine: 0.10% | L-Valine: 0.10% | L-Valine: 0.10% |
| | Total: 0.1% | Total: 0.1% | Total: 0.1% |
| 1 | ¹ — | — | — |
| 3 | L-Valine: 0.09% | L-Valine: 0.09% | L-Valine: 0.09% |
| | Total: 0.1% | Total: 0.1% | Total: 0.1% |
| 6 | L-Valine: 0.13% | L-Valine: 0.13% | L-Valine: 0.13% |
| | Total: 0.1% | Total: 0.1% | Total: 0.1% |
| 9 | L-Valine: 0.19% | L-Valine: 0.19% | L-Valine: 0.19% |
| | Total: 0.2% | Total: 0.2% | Total: 0.2% |
| 12 | — | — | — |
| 18 | L-Valine: 0.20% | L-Valine: 0.20% | L-Valine: 0.20% |
| | Total: 0.2% | Total: 0.2% | Total: 0.2% |

¹ Not measured.

The amount of water in the samples is shown in Table 10.

TABLE 10
Water content of IR/MR pharmaceutical
granulation following storage

| | | Long Term | Accelerated |
|---|---|---|---|
| Time | Refrigerated | 25 ± 2° C./ | 40 ± 2° C./ |
| (months) | 5 ± 3° C. | 60 ± 5% RH | 75 ± 5% RH |
| Initial | 0.16% | | |
| 0.5 | ¹ — | — | 0.39% |
| 1 | 0.30% | 0.23% | 0.20% |
| 3 | 0.17% | 0.22% | 0.26% |
| 6 | 0.21% | 0.20% | 0.24% |
| 9 | — | 0.15% | — |
| 12 | 0.15% | 0.16% | — |
| 18 | — | — | — |
| 24 | 0.26% | 0.27% | — |

¹ Not measured.

Dissolution profiles for the pharmaceutical granulation samples stored under conditions (1) and (2) is shown in FIGS. 10A and 10B, respectively. The profiles represent 0, 3, 6, 9, 12, and 18 months of storage at the test condition. The dissolution profiles were determined as described in Example 2. The dissolution media contained 3,164 mg of the pharmaceutical granulation in 500 mL of water.

A similar stability study was undertaken for a pharmaceutical composition comprising the excipient granulation (EG), immediate release pharmaceutical granulation (IR), and the modified release granulation (MR). The granulations were combined, and 4 grams of the pharmaceutical composition was placed into a high barrier pouch (Amcor RFA-045, Specification No. 91038). The samples were stored under test conditions (1)-(3) for up to 24 months. The samples contained about 7.6 wt % of the excipient granules of Example 3, about 25.2 wt % of the immediate release pharmaceutical granules of Example 1, and about 67.2 wt % of the modified release pharmaceutical granules of Example 2.

The 4-((L-valyl)oxy)butanoic acid content following storage is shown in Table 11, the impurity content in Table 12, the water content is shown in Table 13, and the reconstitution time in Table 14. The 4-((L-valyl)oxy)butanoic acid and impurity content were determined using HPLC, the water content according to USP <921> Method 1c, and the reconstitution time by visual assessment.

TABLE 11
4-((L-Valyl)oxy)butanoic acid content of EG/IR/MR
pharmaceutical compositions following storage.

| | | Accelerated | Stress |
|---|---|---|---|
| Time | Long-term | 25 ± 2° C./ | 40 ± 2° C./ |
| (months) | 5 ± 3° C. | 60 ± 5% RH | 75 ± 5% RH |
| Initial | 99.0% | | |
| 1 | ¹ — | — | 98.1% |
| 3 | 101.0% | 100.1% | 98.3% |
| 6 | 99.7% | 96.7% | 97.7% |
| 9 | 98.8% | 99.0% | — |
| 12 | 97.2% | 97.1% | — |
| 18 | 101.0% | 97.8% | — |

¹ Not measured.

TABLE 12
Impurity content of EG/IR/MR pharmaceutical
compositions following storage.

| | | Accelerated | Stress |
|---|---|---|---|
| Time | Long-term | 25 ± 2° C./ | 40 ± 2° C./ |
| (months) | 5 ± 3° C. | 60 ± 5% RH | 75 ± 5% RH |
| Initial | L-Valine: 0.19% | | |
| | Total: 0.2% ¹ | | |
| 1 | ¹ — | — | L-Valine: 0.38% |
| | | | Total: 0.6%3 |
| 3 | L-Valine: 0.17% | L-Valine: 0.20% | L-Valine: 0.32% |
| | Total: 0.2% | Total: 0.2% | Total: 0.6% |
| 6 | L-Valine: 0.13% | L-Valine: 0.19% | L-Valine: 0.40% |
| | Total: 0.1% | Total: 0.3% | Total: 0.9% |
| 9 | L-Valine: 0.14% | L-Valine: 0.21% | — |
| | Total: 0.1% | Total: 0.3% | |
| 12 | L-Valine: 0.18% | L-Valine: 0.29% | — |
| | Total: 0.2% | Total: 0.4% | |
| 18 | L-Valine: 0.27% | L-Valine: 0.39% | — |
| | Total: 0.3% | Total: 0.6% | |

¹ Not measured.

TABLE 13
Water content of EG/IR/MR pharmaceutical
compositions following storage.

| | | Accelerated | Stress |
|---|---|---|---|
| Time | Long-term | 25 ± 2° C./ | 40 ± 2° C./ |
| (months) | 5 ± 3° C. | 60 ± 5% RH | 75 ± 5% RH |
| Initial | 0.31% | | |
| 1 | ¹ — | — | 0.22% |
| 3 | 0.26% | 0.25% | 0.29% |
| 6 | 0.38% | 0.46% | 0.40% |
| 9 | 0.32% | 0.37% | — |
| 12 | 0.17% | 0.18% | — |
| 18 | 0.51% | 0.60% | — |

¹ Not measured.

TABLE 14
Reconstitution time of EG/IR/MR pharmaceutical
compositions following storage.

| | | | |
|---|---|---|---|
| | | Accelerated | Stress |
| Time | Long-term | 25 ± 2° C./ | 40 ± 2° C./ |
| (months) | 5 ± 3° C. | 60 ± 5% RH | 75 ± 5% RH |

| | |
|---|---|
| ² Initial | 2 min 24 s |

| ³ 1 | ¹ — | — | | 15 s |
|---|---|---|---|---|
| ³ 3 | 15 s | 17 | s | 17 s |
| ³ 6 | 15 s | 15 | s | 15 s |
| ³ 9 | 15 s | 15 | s | — |
| ³ 12 | 15 s | 1 5 | s | — |
| ³ 18 | 15 s | 15 | s | — |

¹ Not measured.
² Value based on time when suspension appeared to reach maximum reconstitution.
³ Value based on time when suspension initially appeared reconstituted.

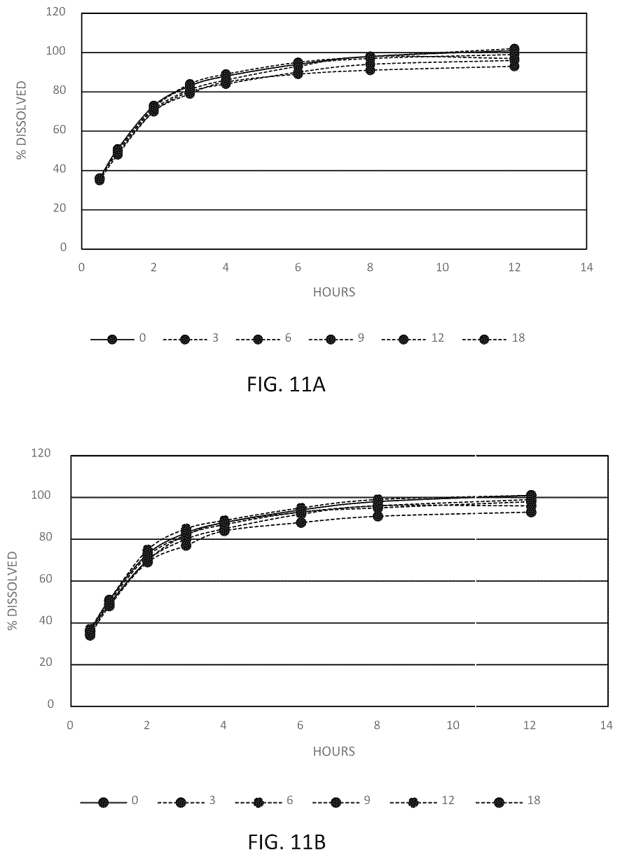

Dissolution profiles for pharmaceutical compositions stored under conditions (1) and (2) is shown in FIGS. 11A and 11B, respectively. The profiles represent 0, 3, 6, 9, 12, and 18 months of storage at the test condition. The dissolution profiles were measured using an Agilent dissolution bath and autosampler equipped with Apparatus II paddles. The dissolution media was made by combining and mixing 2.99 g of sodium acetate trihydrate, 1.2 mL of glacial acetic acid, and 1,000 mL of water, with the pH adjusted to 4.5±0.05 using acetic acid. Five-hundred (500) mL of the dissolution media was transferred to a vessel and the temperature equilibrated to 37° C.±0.5° C. A pharmaceutical composition (3,164 mg) was added with the paddles spinning at 75 rpm. Samples were taken at intervals and the content of 4-((L-valyl)oxy)butanoic acid measured using HPLC.

It should be noted that there are alternative ways of implementing the embodiments disclosed herein. Accordingly, the present embodiments are to be considered as illustrative and not restrictive. Furthermore, the claims are not to be limited to the details given herein and are entitled their full scope and equivalents thereof.

What is claimed is:

1. A pharmaceutical composition comprising:
a pharmaceutical granulation comprising 4-((L-valyl)oxy)butanoic acid; and
an excipient granulation.

2. The pharmaceutical composition of claim 1, wherein,
the pharmaceutical composition comprises from 2 wt % to 12 wt % of the excipient granulation; and
wt % is based on the total weight of the pharmaceutical composition.

3. The pharmaceutical composition of claim 1, wherein,
the pharmaceutical composition comprises from 86 wt % to 98 wt % of the pharmaceutical granulation; and
wt % is based on the total weight of the pharmaceutical composition.

4. The pharmaceutical composition of claim 1, wherein the pharmaceutical granulation comprises:
an immediate release pharmaceutical granulation comprising a plurality of immediate release pharmaceutical granules comprising 4-((L-valyl)oxy)butanoic acid; and
a modified release pharmaceutical granulation comprising a plurality of modified release pharmaceutical granules comprising 4-((L-valyl)oxy)butanoic acid.

5. The pharmaceutical composition of claim 1, wherein,
the excipient granulation comprising a plurality of excipient granules; and
the excipient granules comprise:
a viscosifying agent;
a disintegrant; and
one or more additional excipients.

6. The pharmaceutical composition of claim 5, wherein the excipient granulation comprises:
from 2 wt % to 22 wt % of the binder;
from 10 wt % to 30 wt % of the diluent;
from 1 wt % to 20 wt % of the filler;
from 15 wt % to 35 wt % of the flavoring agent;
from 0.5 wt % to 6.5 wt % of the buffer;
from 0.5 wt % to 8 wt % of the acid; and/or
from 0.1 wt % to 6 wt % of the sweetening agent,
wherein wt % is based on the total weight of the excipient granules.

7. The pharmaceutical composition of claim 5, wherein the excipient granulation comprise:
from 1 wt % to 35 wt % of the viscosifying agent; and
from 1 wt % to 25 wt % of the disintegrant;
wherein wt % is based on the total weight of the excipient granules.

8. The pharmaceutical composition of claim 5, wherein following storage at 5±3° C. or at 25±2° C./60±5% RH sealed in high barrier pouch for 18 months, the 4-((L-valyl)oxy)butanoic acid dissolution profile is similar to the 4-((L-valyl)oxy)butanoic acid dissolution profile prior to storage.
Following storage of a pharmaceutical composition provided by the present disclosure at 5±3° C. or at 25±2° C./60±5% RH sealed in high barrier pouch for 24 months, the reconstitution time is about 15 seconds.

9. The pharmaceutical composition of claim 5, wherein following storage at 5±3° C. or at 25±2° C./60±5% RH sealed in high barrier pouch for 18 months, the 4-((L-valyl)oxy)butanoic acid content is greater than 96% of the 4-((L-valyl)oxy)butanoic acid prior to storage.

10. The pharmaceutical composition of claim 5, wherein following storage at 5±3° C. in a sealed in high barrier pouch for 18 months, the total impurity content is 0.3% or less and following storage at or at 25±2° C./60±5% RH sealed in high barrier pouch for 18 months, the total impurity content is 0.6% or less.

11. The pharmaceutical composition of claim 5, wherein following storage at 5±3° C. in a sealed high barrier pouch for 18 months, the L-valine impurity content is 0.3% or less and following storage at or at 25±2° C./60±5% RH sealed in high barrier pouch for 18 months, the L-valine impurity content is 0.4% or less.

12. A pharmaceutical product comprising the pharmaceutical composition of claim 1.

13. A kit comprising:
the pharmaceutical composition of claim 1; and
and instructions for using contents of the kit.

14. An oral pharmaceutical composition prepared by combining and mixing the pharmaceutical composition of claim 1 with water.

15. The oral pharmaceutical composition of claim 14, wherein the oral pharmaceutical composition comprises:
a viscous aqueous solution; and
a modified release granulation suspended in the viscous aqueous solution.

16. The oral pharmaceutical composition of claim 14, wherein the viscous aqueous solution has a viscosity from 50 cP to 1,000 cP at a temperature at 23° C., wherein the viscosity is determined according to USP <912> Method 1c using a No. 62 spindle at 12 rpm, at a concentration of 0.30 to 0.42 g/mL.

17. A method of treating a disease in a patient comprising orally administering to a patient in need of such treatment a therapeutically effective amount of the oral pharmaceutical composition claim 14.
